(19) United States
(12) Patent Application Publication
(10) Pub. No.: US 2025/0109386 A1
(43) Pub. Date: Apr. 3, 2025

(54) HYPOIMMUNOGENIC BETA CELLS AND METHODS OF PRODUCING THE SAME

(71) Applicant: President and Fellows of Harvard College, Cambridge, MA (US)

(72) Inventors: Douglas A. Melton, Cambridge, MA (US); Elad Sintov, Cambridge, MA (US)

(21) Appl. No.: 18/802,111

(22) Filed: Aug. 13, 2024

Related U.S. Application Data
(63) Continuation of application No. PCT/US2023/013246, filed on Feb. 16, 2023.
(60) Provisional application No. 63/332,302, filed on Apr. 19, 2022.
(60) Provisional application No. 63/311,037, filed on Feb. 16, 2022.

(51) Int. Cl. C12N 5/071

(52) U.S. Cl. CPC C12N 5/0676; C12N 2510/00

(57) ABSTRACT

Disclosed herein are hypoimmunogenic stem cell-derived β cells for use in various applications, such as cell therapy.

### RELATED APPLICATIONS

This application is a continuation Application of International Application No. PCT/US2023/013246, filed Feb. 16, 2023, which claims the benefit of and priority to U.S. Provisional Application No. 63/311,037, filed Feb. 16, 2022, and U.S. Provisional Application No. 63/332,302, filed Apr. 19, 2022. The entire teachings of the applications are incorporated herein by reference. International Application No. PCT/US2023/013246 was published under PCT Article 21(2) in English.

### BACKGROUND OF THE INVENTION

One hundred years ago, the first type 1 diabetes (T1D) diabetic patient was treated in Toronto with a “pancreatic extract,” part of a series of experiments that later led to the discovery of insulin (Banting et al., 1922). Since then, the scientific basis for T1D has been described as an autoimmune attack of pancreatic insulin-producing β-cells. However, despite technological advances, such as insulin pumps and continuous glucose monitoring devices (Kovatchev, 2019), for a long time exogenous insulin administration remained the only option to regulate blood glucose levels. Nevertheless, in the last few decades β-cell replacement strategies emerged as a new hope for patients, beginning when cadaveric islet transplantations demonstrated insulin independence (Shapiro et al., 2000) and followed by the exploration of human pluripotent stem cells (hPSC) as an unlimited source for beta-cell differentiation and replacement.

Despite these advances, a major challenge remaining is protecting SC-islets from an allogeneic response and recurring β-cell destruction due to autoimmunity. The use of immunosuppressor drugs can lead to complications and may lead to graft impairment in the long term (Lehmann et al., 2008). Encapsulation methods and cell capturing devices are still in development (Alagpulinsa et al., 2019). Modulation of the immune system by regulatory T cells has been suggested (Raffin et al., 2020), and great effort has been dedicated recently to genetically modifying hPSCs to create donor universal lines for cell replacement therapies that could be transplanted ‘naked’ without the use of immunosuppressor drugs. However, these naked universal lines are still in development.

### SUMMARY OF THE INVENTION

A comprehensive analysis was executed to define forces that trigger immune attack of an endocrine tissue in various modeling systems. For the first time in human SC-islets, new genes were explored to serve as potential targets for gene editing to assist cells in evading allogeneic attack. The focus of the assessment was to determine the main transcriptional events that take place in an inflammatory environment caused by introducing SC-islets to an allogeneic immune system, and the ultimate strategy to reverse/prevent them with as little as a single perturbation. By using single cell RNA sequencing (scRNA-seq) and whole genome CRISPR screens, it was determined that the JAK/STAT type-II interferon (IFN) pathway is the leading modulator of early and late inflammatory response events in vivo and in vitro. It was determined that while manipulation of upstream and central mediators of this pathway allow some reduction of SC-islet immunogenicity, one unique approach is to target downstream components with C-X-C motif chemokine ligand 10 (CXCL10) depletion.

Disclosed herein are modified beta cells, e.g., modified stem cell-derived beta cells. The modified beta cells may comprise one or more perturbations in the JAK/STAT Type II interferon (IFN) pathway.

In some embodiments, the one or more perturbations occur in an IFNγ signaling mediator or a downstream component, e.g., downstream inflammatory component, of the IFN pathway, e.g., via a gene editing system, such as CRISPR. In some embodiments, the one or more perturbations comprise reduced or eliminated expression of one or more downstream components of the IFN pathway. In some embodiments, the one or more perturbations comprise reduced or eliminated expression of one or more genes listed in Table 1. In some embodiments, the one or more perturbations comprise reduced expression of one or more genes selected from the group consisting of CXCL10, STAT1, and TAP1.

Also disclosed herein are modified beta cells, e.g., stem cell-derived beta cells, which do not express (i.e., have been modified to lack expression of) CXCL10. Also disclosed herein are modified beta cells, e.g., stem cell-derived beta cells, which do not express (i.e., have been modified to lack expression of) STAT1.

In some embodiments, the modified beta cell further exhibits increased or activated expression of one or more immunomodulatory factors. In some embodiments, the one or more immunomodulatory factors are selected from Table 2. In one embodiment, the one or more immunomodulatory factors are selected from the group consisting of PD-L1, CD47, SOCS1, and HLA-E.

In some embodiments, the modified beta cell is a hypoimmunogenic beta cell, e.g., a hypoimmunogenic stem cell-derived beta cell. In some embodiments, the modified beta cell exhibits: one or more of auto and/or allogeneic rejection protection; increased cell survival under immune rejection; and/or decreased T cell activation and/or NK cell activation upon transplant. In some embodiments, the beta cell is a human cell or a non-human cell, e.g., a mouse cell.

Disclosed herein are methods for producing a hypoimmunogenic beta cell, e.g., a hypoimmunogenic stem cell-derived beta cell, comprising dampening or silencing the IFN pathway of the beta cell.

In some embodiments, the methods comprise reducing or eliminating expression of one or more genes selected from Table 1. In some embodiments, the methods comprise reducing or eliminating expression of one or more genes selected from the group consisting of CXCL10, STAT1, and TAP1, for example eliminating expression of CXCL10 or STAT1.

Also disclosed herein are methods for producing a hypoimmunogenic beta cell, e.g., a hypoimmunogenic stem cell-derived beta cell, comprising perturbing one or more downstream components of the IFN pathway of the beta cell.

In some embodiments, the perturbation comprises reducing or eliminating expression of one or more downstream components of the IFN pathway, e.g., using a gene editing system, such as CRISPR. In some embodiments, the one or more downstream components are selected from Table 1. In one embodiment, the one or more downstream components are selected from the group consisting of CXCL10, STAT1, and TAP1; one embodiment comprises eliminating expression of CXCL10 or STAT 1.

In some embodiments, the hypoimmunogenic beta cell exhibits: one or more of auto and/or allogeneic rejection protection; increased cell survival under immune rejection; and/or decreased T cell activation and/or NK cell activation upon transplant.

In some embodiments, the methods further comprise increasing or activating expression of one or more immunomodulatory factors. In some embodiments, the one or more immunomodulatory factors are selected from Table 2. In one embodiment, the one or more immunomodulatory factors are selected from the group consisting of PD-L1, CD47, SOCS1, and HLA-E.

In some embodiments, the hypoimmunogenic beta cell is a human cell or a non-human cell, e.g., a mouse cell.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1J demonstrate single cell transcriptional profile of SC-islet graft rejection in a humanized model. FIG. 1A shows SC-islets were transplanted under the kidney capsule of MHC^{null} NSG mice. After 2-4 weeks graft function was assessed by human insulin detection in mouse serum, 30 minutes after glucose injection. Half of the mice were then injected with human PBMCs, and human insulin was continuously monitored until graft failure was observed. Grafted cells were then extracted and analyzed by 10×scRNA-seq for gene expression. FIG. 1B provides representative images of transplanted kidneys after 10 weeks with or without PBMC injection. Bar=2 mm. FIG. 1C provides IVIS imaging of control and humanized mice transplanted with GAPDH-luciferase SC-islets at week 1 and 8 after PBMC injection. FIG. 1D shows SC-islet graft rejection assessed by IVIS imaging quantitation of emitted luminescence (top panel—n=6 mice) or blood human insulin detection by ELISA (bottom panel), over time after PBMC injection, 30 min after glucose injections to the overnight fasted mice. n=15 mice. FIG. 1E shows immunofluorescent staining of kidney SC-islet grafts sections from either humanized or control mice, 6 wks after PBMC injections. Bars are 10 m in main panels and 3 m in magnified panels. Kidney (K) and graft (G) margins are outlined. CHGA=chromogranin A. FIG. 1F shows flow cytometry of SC-α (Glucagon+C-peptide-) and SC-β (Glucagon-C-peptide+) in extracted grafts at week 10 post PBMCs H-K) 10×scRNA-seq analysis. FIG. 1G shows flow cytometry of human T-lymphocytes in humanized mouse blood, spleen and graft infiltrating at week 10 post PBMC injection. % human CD45+ are gated from mouse CD45 negative population. % CD4+ and % CD8+ are gated from hCD45+/hCD3+. FIG. 1H provides UMAP plots of human graft cells extracted from mice after 10 weeks with or without PBMC injection. Integration of n=6 mice per group. SC-endocrine cell clusters are indicated. FIG. 1I provides volcano plots of differential expressed genes in SC-β and SC-α in rejected (+PBMCs) compared to unrejected (no PBMCs) grafts. FIG. 1J provides violin plots of selected genes, expressed in SC-Endocrine cells, associated with the IFNγ response.

FIGS. 2A-2J demonstrate immune challenged SC-islets first response profiled by single cell transcription analysis after co-culture with human allogeneic PBMCs. FIG. 2A shows enriched (CD49A magnetic sorting) human hESC derived SC-islets, pre-treated with thapsigargin (5 m for 5 hrs), that were co-cultured with human allogeneic PBMCs (n=2 donors) for 0, 24 and 48h and 10×scRNA-seq was performed. FIG. 2B provides dot plots representing expression of activation/inhibitory genes in specific T cell and NK cell populations, in response to a 0, 24 and 48h stimulation with SC-islets. FIGS. 2C-2D provide volcano plots of differential expressed genes in SC-α (FIG. 2C) and SC-β (FIG. 2D) after 24 hr co-culture with PBMCs compared to control (t=0, without co-culture). FIG. 2E shows top results of pathway analysis and Gene Set Enrichment Analysis (GSEA) of upregulated genes in co-cultured SC-β after 48 hrs. FIG. 2F shows expression of selected inflammatory genes in SC-α and SC-β, in response to a 0, 24 and 48h co-culture with PBMCs. FIG. 2G provides violin plots of SC-β timed expression of selected genes. FIG. 2H shows ELISA for human CXCL10, from supernatant of 24/48 hrs of co-culture of SC-islets and PBMCs. n=2 donors FIGS. 2I-2J). FIGS. 2I-2J shows immunofluorescent staining of CXCL10 (FIG. 21) and phosphorylated STAT1 (FIG. 2J) in SC-islet clusters 48 hrs after treatment with 20 ng/ml rhIFNγ. C-peptide staining for SC-β. Bars are 10 m in main panels and 2 m in magnified panels.

FIGS. 3A-3D demonstrate that a whole genome CRISPR screen identifies tolerizing perturbations in SC-islet grafts under allogeneic rejection. FIG. 3A shows SC-islets were transduced with a pooled lentivirus CRISPR library (Brunello) that express Cas9 and 77,441 gRNAs in pLentiCRISPRv2. SC-islets were then transplanted as previously described into a humanized mouse system (see FIG. 2). Upon failure/rejection, grafts were retrieved, and genomic DNA was extracted for sequencing. FIG. 3B shows SC-islet graft rejection measured by blood human insulin and C-peptide detection by ELISA over time after PBMC injection, 30 min after glucose injections to the overnight fasted mice. n=6 per group. FIG. 3C provides an analysis of enriched and depleted genes knockouts by mean fold changes of gRNA counts. Ranking of gene knockouts are shown as Gene to Environment interaction where the y-axis is the interaction between the tolerance to PBMC and the knockout, while the x-axis is the KO effect across environments. FIG. 3D provides violin plots presenting individual gRNAs normalized counts from mice replicates (n=6 per condition) of IFN associated genes with positive and negative enrichment in screen. Compared between conditions (±PBMC) and to control gRNAs (n=4 random control gRNAs). Red line in every violin represents median values. Dashed line represent mean of intergenic gRNA log 2norm counts. Error bars or shaded areas are mean±SD. *p<0.05, **p<0.01 unpaired two-tailed t-test.

FIGS. 4A-4G demonstrate perturbation of individual components of IFNγ pathway in search of potential gene targets to diminish activation of PBMCs and enhance survival of co-cultured SC-β. FIG. 4A provides venn diagrams featuring significantly upregulated genes (log 2 fold change >1 and adj pvalus<0.05) obtained from in in vivo (blue) and in vitro (red) co-culture scRNA-seq data that are common to CRISPR screen hits (positively enriched in humanized mice, log 2 fold change >1) (green). Right diagrams are genes from SC-α populations, while right is from SC-β populations. FIG. 4B shows human hESC derived SC-islets, pre-transduced with lentiviruses carrying Cas9+gRNA (KO in pLentiCRISPRv2) or a given ORF insert (OE in pLX_317), pre-treated with thapsigargin (5 m for 5 hrs) and were co-cultured with human allogeneic PBMCs. FIG. 4C provides flow cytometry analysis for % TUNEL+(apoptotic)SC-islet cells and SC-β (C-peptide+), following 48 hr PBMC co-culture. % Apoptosis was calculated by fraction from baseline (no PBMC added). gRNA lentivirus transduced SC-islets were compared to Non-targeting (NT) gRNA, and ORF overexpression (OE) transduced SC-islets were compared to eGFP OE. n=2-3 PBMC donors. FIG. 4D shows transduced SC-islets were cocultured with Cell Trace Violet (CTV) labeled PBMC for 48 hrs. PBMCs were then separated and allowed to grow in culture for an additional 7 days, followed by CD3 staining for flow cytometry. CD3+ were gated for the CTV negative fraction of divided cells. PBMCs treated with anti-CD3/CD28 activation beads served as positive control. n=2-7 donors. FIG. 4E shows blocking antibody experiment where PBMC were pre-treated with neutralizing anti-CXCR3, or SC-islets were pretreated with anti-TLR4, or anti-CXCL10 was added to co-culture media. FIG. 4F provides flow cytometry analysis for % TUNEL+SC-β (C-peptide+), following 48 hr PBMC co-culture. n=2-6 donors. FIG. 4G provides % proliferated CD3 T-cell measured by CTV negative T-cells following co-culture. n=3 donors. Error bars are mean±SD. *p<0.05, **p<0.01, ***p<0.001, ****p<0.0001 unpaired two-tailed t-test.

FIGS. 5A-5K demonstrate generation and performance of CXCL10 KO and STAT1 KO hESC lines. FIGS. 5A-5B provides schematic presentations of targeting the CXCL10 (FIG. 5A) or STAT1 (FIG. 5B) locus by nucleofection of hESCs with Cas9/sgRNA ribonucleoprotein complex (RNP) and a targeting vector containing site specific homology arms (HA) as indicated. Red and blue arrows show PCR primer locations used for clonal genotyping as shown in FIG. 11. FIG. 5C shows % SC-β in multiple batches of C10G and STIL differentiations compared to control wild type (WT) and GAPDH-luc (GL) lines. n=1-3 differentiations. FIG. 5D shows SC-islet GSIS function assay of different lines, 12-15 weeks after transplantation into NSG-mice. Results presented as stimulation ratios of blood human insulin (ELISA) before and after glucose injection (2 g/kg). FIG. 5E provides flow cytometry analysis of intracellular CXCL10 protein in WT/C10G SC-islets and SC-β (C-peptide+)±rhIFNγ 48 hr treatment and 5 hr monensin to prevent protein secretion. n=1-3. FIG. 5F shows CXCL10 ELISA assay to supernatants from ±rhIFNγ treated WT/C10G/ST1L SC-islets. Dashed line is the lower detection limit. n=2-3 differentiations. FIG. 5G provides a flow cytometry analysis for protein expression in rhIFNγ GL/ST1L SC-islets as indicated. n=3-4. FIG. 5H shows gene modified (GM; C10G/ST1L) and control (WT/GL) lines were differentiated into SC-islets, pre-treated with thapsigargin (5 m for 5 hrs) and co-cultured with human PBMCs or purified T-cells/NK cells. FIGS. 5I-5K show flow cytometry was used to assess % TUNEL+C-peptide+ WT or C10G SC-β cells (n=3-5 donors) (FIG. 5I), GL or ST1L SC-β cells (n=2-3 donors) (FIG. 5J) and % proliferated CD3 T-cell measured by CTV negative T-cells following co-culture as indicated (n=2-4 donors) (FIG. 5K).

FIGS. 6A-6B demonstrate CXCL10 KO SC-islet grafts evade allo-rejection in humanized mice. FIG. 6A shows WT or C10G SC-islets were transplanted under the kidney capsule of MHC^{null} NSG mice (n=10 from each line). After 4 weeks graft function was assessed by human insulin detection in mouse serum, 30 minutes after glucose injection. 6-7 mice from each group were then injected with human PBMCs (n=2 human donors), while the rest served as control non-humanized. Human insulin was continuously monitored until graft failure was observed. FIG. 6B shows SC-islet graft rejection measured by blood human insulin detection by ELISA over time after PBMC injection, 30 min after glucose injections to the overnight fasted mice. Error bars are mean±SD. *p<0.05, **p<0.01, unpaired two-tailed t-test.

FIGS. 7A-7F demonstrate single cell transcriptional profile of SC-islet graft rejection in a humanized model. FIG. 7A shows immunofluorescent staining of kidney SC-islet grafts sections from either humanized or control mice, 6 wks after PBMC injections. Bars are 10 m in main panels and 3 m in magnified panels. Kidney (K) and graft (G) margins are outlined. CHGA=chromogranin A. FIG. 7B provides cell counts of cell populations from endocrine scRNAseq analysis for FIGS. 1H-1I. FIG. 7C shows cell markers for endocrine cells. FIG. 7D shows differential expression of selected genes in various cell populations (SC-β cells, SC-α cells, and SC-EC cells). FIG. 7E provides pathway analysis of upregulated genes in various cell populations (SC-β cells, SC-α cells, and SC-EC cells). FIG. 7F provides a violin plot of CXCL10 expressed in SC-Endocrine cells.

FIGS. 8A-8F demonstrate immune challenged SC-islets first response profiled by single cell transcription analysis after co-culture with human allogeneic PBMCs. FIG. 8A provides UMAPs of in vitro PBMC and SC-islet co-cultures by time (see FIG. 2). FIG. 8B provides UMAPs of in vitro PBMC and SC-islet co-cultures by cell type (see FIG. 2). FIG. 8C provides dot blots representing marker genes used to define cell types in the analysis. FIG. 8D shows pathway analysis of upregulated genes in different cell populations. FIG. 8E shows GSEA analysis of differential expressed genes in SC-beta cells. FIG. 8F shows expression of inflammatory genes in in different cell populations in response to a 0, 24 and 48h co-culture with PBMCs.

FIGS. 9A-9C demonstrate that a whole genome CRISPR screen identifies tolerizing perturbations in SC-islet grafts under allogeneic rejection. FIG. 9A provides a gating strategy used for flow cytometry of humanized mice blood to detect human T-cells. FIG. 9B shows detection of human T cells of humanized mice blood over the course of the experiments described in FIG. 3. FIG. 9C shows human insulin and C-peptide detected by ELISA at the end of the experiment in non-fasted mice (see FIG. 3B).

FIGS. 10A-10E demonstrate perturbation of individual components of IFNγ pathway in search of potential gene targets to diminish activation of PBMCs and enhance survival of co-cultured SC-β. FIG. 10A shows target proteins' expression after lentiviral transductions with non-targeting (NT) or targeting (as indicated) gRNAs. FIGS. 10B-10C show T cell activation marker expression, CD25 (FIG. 10B) or CD69 (FIG. 10C), 48 hrs after co-culture with pre-transduced SC-islets. FIG. 10D shows CXCL10 detection in cell supernatants after 48h co-cultures. FIG. 10E shows T cell activation marker expression (CD25 or CD69), 48 hrs after co-culture with blocking antibodies.

FIGS. 11A-11J demonstrate generation and performance of CXCL10 KO and STAT1 KO hESC lines. FIG. 11A shows clonal genotyping of endogenous or targeted allelles. Endogenous amplified PCR bands were isolated and sequenced for detection of indels, shown in dashed blue frames. FIG. 11B provides karyotyping of G10G and STIL. FIG. 11C provides pluripotent marker expression in all 4 lines: WT, C10G, GL and ST1L. FIG. 11D provides flow cytometry analysis to assess % SC-β (% C-peptide+/NKX6.1+ or % C-peptide+/glucagon−) in C10G and STIL hESC at stage 6 of the Melton β-cell differentiation protocol. FIGS. 11E-11F show relative light units (RLU) detected by luminescence plate reader, of ST1L hESC in different stages (S0=before ES stage) of β-cell differentiation, in increasing number of cell/well. FIG. 11G shows flow cytometry was used to assess % TUNEL+C-peptide+ WT or C10G SC-islets (n=3-5 donors) and GL or ST1L SC-islets (n=2-3 donors). FIGS. 11H-11J show T cell activation marker expression (CD25 or CD69), 48 hrs after co-culture. FIG. 11J provides NK cell activation marker expression (CD107a), 48 hrs after co-culture.

FIG. 12 shows absolute values of insulin detected by ELISA, as shown in FIG. 6B.

FIG. 13 provides a flowchart of the differentiation stages for obtaining stem cell-derived 0 cells.

FIG. 14 demonstrates single cell profiling of in vitro alloimmune responses in vitro. A diagram exemplifies the process for the in vitro co-culture of ESC derived SC-islets and performing scRNA-seq, which reveals potential targeting genes to improve cell survival under immune rejection (IFN pathway “alarming” genes). UMAP projections are provided for various cell types and across different time periods.

FIG. 15 shows an alarm gene activation plot across t=0, 24 hours, and 48 hours for +PBMC-2 and +PBMC-1 with different agents and demonstrates immune attacked allo-SC-β upregulate alarm genes from the interferon pathway.

FIG. 16 demonstrates single cell profiling of autoimmune responses in vitro. A diagram exemplifies the process for the single cell profiling of autoimmune responses in vitro from a Type-I diabetes patient. UMAP projections are provided for different cell types and comparing co-cultured iPSC-beta cells with control iPSC-beta cells.

FIG. 17 demonstrates immune attacked autologous iPSC-β cells upregulate alarm genes in the interferon pathway. In vitro co-cultures of T1D derived iPSC-islets and scRNA-seq reveal potential targeting genes in order to improve cell survival under immune rejection (IFN pathway “alarming” genes). The UMAP projection compares Alarmed iPSC-beta with iPSC-beta cells and changes based on the targeted genes.

FIG. 18 provides a diagram identifying potential targets for affecting SC-islet immunogenicity, including: CXCL9, CXCL10, CXCL11 depletion as JAK/STAT downstream effectors; IFNGR1, STAT1, IRF1, JAK1/2 depletion as JAK/STAT as upstream regulators; SOCS1 expression; and TNFSF10 and TNFRSF12A as part of the TNF apoptosis pathway. B2M depletion and PD-L1/CD274 expression control perturbations, which are known to have an effect in SC-islets.

FIG. 19 shows a target list of genes that may be depleted or expressed for producing hypoimmunogenic SC-islets. The targets are identified using scRNA sequencing of SC-iselt co-cultures with allo-PBMCs.

FIG. 20 provides a diagram of an in vivo whole-genome pooled CRSPR screen model. Whole genome CRISPR survival screens in humanized mice revealed potential targeting genes (IFN pathway “alarming” genes) that improve cell survival under immune rejection.

FIG. 21 shows SC-islet allograft survival screens for post-glucose mice comparing SC-islets Tx with and without PMBCs measuring human insulin and human C-peptide over time as well as percentage of human CD45 and CD4 and CD8 expression over time post SC-islet Tx.

FIG. 22 shows results of an SC-islet allo-graft survival screen with enrichment by gRNA. Average gRNA counts comparing mice with no PBMCs (control) and with PBMCs based on the agents used. Whole genome CRISPR survival screens in humanized mice, reveal potential targeting genes, to improve cell survival under immune rejection, including IFN pathway “alarming” genes.

FIG. 23 shows a target list of genes that may be depleted or expressed for producing hypoimmunogenic SC-islets. The targets are identified using a CRISPR survival screen in H. mice.

FIG. 24 demonstrates CXCL10 and STAT1 inhibition reduces SC-islet immunogenicity. CXCL10 and STAT1 inhibition using CRISPR KO shows a reduction in activation/proliferation of T-cells co-cultured with modified SC-islets and an improvement in survival of modified SC-islets. The plots demonstrate CXCL10 and STAT1 inhibition using CRISPR KO by measuring percentage proliferated CD3+ T cells and percentage live SC-β. A co-culture diagram demonstrates lentivirus transduction with gRNA and CRISPR/Cas9 with SC-islets and allo-PMBCs and percentage CXCL10 and STAT1 measurements for IFNγ expression when comparing non-targeting gRNA with CXCL10/STAT1 gRNA.

FIG. 25 shows expression of immune regulators in SC-islets. SOCS1 overexpression was shown to improve the survival of modified SC-islets and reduce chemokine secretion (CXCL10), the effect of which is comparable to PD-L1 overexpression. The plots provided show percent of apoptotic SC-β cells for eGFP, PD-L1, and SOCS1 overexpression, as well as showing empty vector versus SOCS1 overexpression.

FIG. 26 demonstrates the design of CXCL10 (left) and STAT1 (right) knockout hESC lines.

FIG. 27 demonstrates improved survival of CXCL10 KO SC-islets with co-cultured immune cells. The percentage of apoptotic cells for PBMCs, T cells, and NK cells with WT co-culture versus C10G2 co-culture is shown.

FIG. 28 shows CXCL10 expression and secretion for WT, CXCL10 KO, and STAT1 KO SC-islets and SC-islets+IFNγ.

FIG. 29 demonstrates effects of the STAT1 KO luciferase line (ST1L). STAT1 and HLA expression are shown for Hues8 cells+IFNγ. Also shown is luciferase activity in vitro and in vivo.

FIG. 30 provides an analysis of CXCL10 knockout cell lines (C10G). Fluorescence plots for GM SC Islets for CXCL10 knockout line (C10G) are shown, as well as graphs showing the percentage CXCL10.0 expression of SC-islets and SC-islets+IFNγ for wild-type Hues8 and CXCL10 knockout Hues8.

FIG. 31 provides an analysis of STAT1 knockout luciferase cell lines (ST1L). C-peptide/NKX6.1 plots are shown for STL15.1 and STL15.2. Also provided is a graph showing percentage of cells expressing wild type versus ST1L15 for STAT1, phos-STAT1, and HLA-ABC cells. Further, luciferase activity of the ST1L cell line is shown in vitro and in vivo.

FIGS. 32A-32B demonstrate that immune tacked iPSC-β cells upregulate alarm genes. FIG. 32A shows a UMAP projection comparing alarmed iPSC-beta with iPSC-beta cells. Activation was exhibited by increases in B2M, HLA-A, and TAP1 and inhibition was exhibited by increases in CD274. FIG. 32B shows log p-values for different enriched pathways in activated endocrine cells.

FIGS. 33A-33B demonstrate T-cell activation was suppressed by gene modified iPSC-β cells. FIG. 33A shows effects of 02M depletion on T cell activation. FIG. 33B shows effects of PD-L1 overexpression on T cell activation.

FIG. 34 shows immune attacked iPSC-β cells express and secrete CXCL10. UMAP projections demonstrate expression of CXCL10 in alarmed iPSC-beta cells vs iPSC-beta cells.

FIGS. 35A-35C demonstrate that CXCL10 inhibition reduces SC-islet immunogenicity. FIG. 35A shows inhibiting CXCL10 is shown to increase percentage of live SC-beta cells and decrease the percentage of apoptotic SC-beta cells. FIG. 35B provides diagrams for producing a co-culture by transduction to form SC-islets and combining with allo-PBMCs as well as a percentage of proliferated CD3+ T cells with non-targeting gRNA and CDCL10 gRNA are provided. FIG. 35C provides a plot comparing intracellular CXCL10 using NT gRNA versus CXCL10 gRNA.

FIG. 36 shows single cell profiling of T-cells noting activation markers/immune checkpoint molecules, apoptosis induction, and nuclear factors for CD4 and CD8 PBMC and SC-cocultures and enriched pathway comparisons of co-cultured vs. unstimulated CD4 T cells and CD8 T cells.

FIG. 37 provides UMAP projections for various cell types and different time periods.

FIG. 38 shows a plot of control vs. treatment expression for various alarm genes over 24 and 48 hours demonstrating co-cultured SC-beta cells upregulate alarm genes (IFN response).

FIGS. 39A-39C demonstrate alarm genes expression over time in co-culture.

FIG. 39A shows CXCL10 secretion in co-culture with SC-islets+PBMCs over 0, 24-hour, and 48-hour time periods. FIG. 39B provides a UMAP projection over different time periods. FIG. 39C provides violin plots of SC-β cell times expression of CXCL10, B2M, and STAT1.

FIGS. 40A-40C demonstrate SC-β cells secrete CXCL10 and stimulate immune cells via CXCR3. FIG. 40A provides a diagram of SC-islets secreting CXCL10, CXCL11, and CXCL9 when targeting T-cells. FIG. 40B provides a diagram demonstrating the co-culture of modified SC-islets and allogeneic PBMCs with anti-CXCR3 Ab. FIG. 40C provides plots measuring % proliferated CD3+ T cells, CD3+CD69+ T cells, and % apoptotic SC-beta cells.

FIG. 41 demonstrates effects of CXCL10 KO cell line on T-cell activation, NK-cell activation, and SC-islet viability.

### DETAILED DESCRIPTION OF THE INVENTION

Human embryonic stem cells (hESCs) provide new opportunities for cell replacement therapy of T1D. Therapeutic quantities of human stem cell-derived islet cells (SC-islets) can be attained in vitro following a stepwise differentiation protocol. Yet, preventing allo-rejection and recurring autoimmunity of grafted cells, without the use of life-long immunosuppressants, remains a major challenge. One actively pursued option is physical encapsulation to prevent direct cell contact with immune cells. Another, pursued here, is transplanting ‘naked’ SC-islet cells that have been genetically modified to evade the immune system. To determine the underlining forces that drive immunogenicity of SC-islets in inflammatory environments, single-cell RNA sequencing (scRNA-seq) was performed of SC-islets, in co-culture with allogeneic perihelial blood mononuclear cells (PBMCs) or under immune attack by PBMCs in humanized mouse transplants. Data analysis identified ‘alarmed’ populations of SC-islet cells and spotted upregulated genes in the interferon (IFN) pathway that contribute to the inflammatory stimulation of co-cultured/infiltrating T-cells. An in vivo whole genome CRISPR screen confirms that targeting IFNγ signaling mediators and downstream inflammatory components has beneficial effects on SC-islet cell survival under immune attack. Manipulating the IFN response by knocking out CXCL10 in SC-islet grafts confers improved survival against allo-rejection compared to wild type grafts in humanized mice. Overall, these results provide insights into the nature of immune destruction of SC-islet during allogeneic responses and provide potential targets for gene editing.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ^{˜}2% become endocrine cells.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods described herein can be performed both in vivo and in vitro.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g. the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The term “pancreatic progenitor” or “pancreatic precursor” are used interchangeably herein and refer to a stem cell which is capable of forming any of pancreatic endocrine cells, pancreatic exocrine cells, or pancreatic duct cells. The term “Pdx1-positive pancreatic progenitor” or “Pdx1+ pancreatic progenitor” as used herein refers to a cell which is a pancreatic endoderm (PE) cell. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. The term “Pdx1-positive, NKX6-1-positive pancreatic progenitor” or “Pdx1+, NKX6-1+ pancreatic progenitor” as used herein refers to a cell which is a pancreatic endoderm (PE) cell. A Pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR.

The terms “stem cell-derived β cell”, “SC-β cell”, and “mature SC-β cell” refer to cells (e.g., pancreatic β cells) that display at least one marker indicative of a pancreatic β cell, express insulin, and display a GSIS response characteristic of an endogenous mature β cell. In some embodiments, the “SC-β cell” comprises a mature pancreatic β cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). Moreover, it should be understood that an SC-β cell of the invention is a non-native, i.e., non-naturally occurring, non-endogenous, cell and has at least one characteristic that is different from a native/naturally-occurring/endogenous cell. Examples of SC-β cells, and methods of obtaining such SC-β cells, are described in WO 2015/002724 and WO 2014/201167, both of which are incorporated herein by reference in their entirety. In some embodiments, the “SC-β cells” are hypoimmunogenic stem cell-derived beta cells, e.g., generate limited or no immune reaction.

The terms “stem cell-derived a cell”, “SC-α cell”, and “mature SC-α cell” refer to cells (e.g., pancreatic a cells) that display at least one marker indicative of a pancreatic a cell, express and secrete glucagon, and display an ultrastructure similar to cadaveric alpha cells. In some embodiments, the “SC-α cell” comprises a mature pancreatic a cell. It is to be understood that the SC-α cells need not be derived (e.g., directly) from stem cells, as the methods of the disclosure are capable of deriving SC-a cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). Moreover, it should be understood that an SC-α cell of the invention is a non-native, i.e., non-naturally occurring, non-endogenous, cell and has at least one characteristic that is different from a native/naturally-occurring/endogenous cell. Examples of SC-α cells, and methods of obtaining such SC-α cells, are described in WO 2019/217487, which is incorporated herein by reference in its entirety.

The terms “stem cell-derived enterochromaffin cell,” “SC-EC cell,” and “mature SC-EC cell” refer to cells (e.g., enterochromaffin cells) that display at least one marker indicative of an enterochromaffin cell, express SLC18A1, and is capable of producing and releasing serotonin (5-HT). In some embodiments, the “SC-EC cell” comprises a mature enterochromaffin cell. It is to be understood that the SC-EC cells need not be derived (e.g., directly) from stem cells, as the methods of the disclosure are capable of deriving SC-EC cells from any progenitor cell using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). Moreover, it should be understood that an SC-α cell of the invention is a non-native, i.e., non-naturally occurring, non-endogenous, cell and has at least one characteristic that is different from a native/naturally-occurring/endogenous cell. Examples of SC-EC cells, and methods of obtaining such SC-EC cells, are described in WO 2019/217493, which is incorporated herein by reference in its entirety.

The term “exocrine cell” as used herein refers to a cell of an exocrine gland, i.e. a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans, that secrete two hormones, insulin and glucagon.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one endocrine cell or a precursor thereof with a maturation factor, or combination of maturation factors) is intended to include incubating the maturation factor and the cell together in vitro (e.g., adding the maturation factors to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one endocrine cell or a precursor thereof with a maturation factor as in the embodiments described herein can be conducted in any suitable manner. For example, the cells may be treated in adherent culture, or in suspension culture. In some embodiments, the cells are treated in conditions that promote cell clustering. The disclosure contemplates any conditions which promote cell clustering. Examples of conditions that promote cell clustering include, without limitation, suspension culture in low attachment tissue culture plates, spinner flasks, or aggrewell plates. In some embodiments, the inventors have observed that clusters have remained stable in media containing 10% serum. In some embodiments, the conditions that promote clustering include a low serum medium.

It is understood that the cells contacted with a maturation factor can also be simultaneously or subsequently contacted with another agent, such as a growth factor or other differentiation agent or environments to stabilize the cells, or to differentiate the cells further.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a stem cell derived cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification), the modification is not suitable for the methods and compositions described herein.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. For example, in the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term a “variant” in referring to a polypeptide could be, e.g., a polypeptide at least 80%, 85%, 90%, 95%, 98%, or 99% identical to full length polypeptide. The variant could be a fragment of full length polypeptide. The variant could be a naturally occurring splice variant. The variant could be a polypeptide at least 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof having an activity of interest. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein.

The term “functional fragments” as used herein is a polypeptide having an amino acid sequence which is smaller in size than, but substantially homologous to the polypeptide it is a fragment of, and where the functional fragment polypeptide sequence is about at least 50%, or 60% or 70% or 80% or 90% or 100% or greater than 100%, for example 1.5-fold, 2-fold, 3-fold, 4-fold or greater than 4-fold effective biological action as the polypeptide from which it is a fragment of. Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g., promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g., EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection,” and the marker is said to be “useful for positive selection.” Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the pancreas or gastrointestinal tract, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years. In some instances, the cells can also be administered subcutaneously, for example, in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion. The phrases “systemic administration,” “administered systemically”, “peripheral administration” and “administered peripherally” as used herein mean the administration of stem cell-derived cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells, germ cells may be used in place of, or with, the stem cells to provide at least one differentiated cell, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges are processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 M forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad 7-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g., a cell not committed to a specific lineage) prior to exposure to at least one maturation factor according to the methods as disclosed herein, whereas in other examples it may be desirable to differentiate the stem cells to one or more intermediate cell types prior to exposure of the at least one maturation factor (s) described herein. For example, the stem cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g., derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hESI (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into stem cell-derived cells did not involve destroying a human embryo.

In another embodiment, the stem cells can be isolated from tissue including solid tissue. In some embodiments, the tissue is skin, fat tissue (e.g., adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g., human, equine, bovine, porcine, canine, feline, rodent, e.g., mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e., acute lymphoid leukemia, acute myeloid leukemia, chronic myeloid leukemia, myelodysplastic syndrome, and neuroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

### Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM P-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Pluripotent SCs can be maintained in an undifferentiated state even without feeder cells. The environment for feeder-free cultures includes a suitable culture substrate, particularly an extracellular matrix such as Matrigel® or laminin. Typically, enzymatic digestion is halted before cells become completely dispersed (say, ^{˜}5 min with collagenase IV). Clumps of ^{˜}10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (^{˜}4,000 rad) primary mouse embryonic fibroblasts, telomerized mouse fibroblasts, or fibroblast-like cells derived from pPS cells. Medium can be conditioned by plating the feeders at a density of ^{˜}5-6×10⁴ cm⁻² in a serum free medium such as KO DMEM supplemented with 20% serum replacement and 4 ng/mL bFGF. Medium that has been conditioned for 1-2 days is supplemented with further bFGF and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

### Methods of Generating Hypoimmunogenic Stem Cell-Derived P Cells

Aspects of the disclosure relate to generating hypoimmunogenic stem cell-derived β cells. Generally, hypoimmunogenic stem cell-derived β cells or precursors thereof, e.g., pancreatic progenitors, can comprise a mixture or combination of different cells, e.g., for example a mixture of cells such as a Pdx1+ pancreatic progenitors, pancreatic progenitors co-expressing Pdx1 and NKX6-1, Ngn3-positive endocrine progenitors, endocrine cells (e.g., β-like cells), and/or other pluripotent or stem cells.

In some embodiments, a somatic cell, e.g., fibroblast can be isolated from a subject, for example as a tissue biopsy, such as, for example, a skin biopsy, and reprogrammed into an induced pluripotent stem cell for further differentiation to produce a stem cell-derived β cell or precursor thereof. In some embodiments, a somatic cell, e.g., fibroblast is maintained in culture by methods known by one of ordinary skill in the art, and in some embodiments, propagated prior to being converted into stem cell-derived β cells.

In some embodiments, the β cells or precursors thereof can be produced according to any suitable culturing protocol to differentiate a stem cell or pluripotent cell to a desired stage of differentiation. In some embodiments, the stem cell-derived β cells or the precursors thereof are produced by culturing at least one pluripotent cell for a period of time and under conditions suitable for the pluripotent cell to differentiate into a stem cell-derived β cell or precursors thereof. In some embodiments, non-native β cells or stem cell-derived β cells (e.g., pancreatic stem cell-derived β cells) may be produced using methods known to those of skill in the art. In certain embodiments, stem cell-derived β cells may be produced using the methods disclosed in WO 2015/002724, WO 2014/201167, WO 2019/217493, and/or WO 2020/247954, all of which are incorporated herein by reference.

In some embodiments, a stem cell is modified to produce a hypoimmunogenic stem cell that is differentiated into a β cell. In some embodiments, a pluripotent or iPS cell is modified prior to differentiating the cell into a hypoimmunogenic β cell. In some embodiments, stem cell-derived β cells obtained by those of skill in the art are further modified to produce hypoimmunogenic stem cell-derived β cells. The stem cell-derived β cells may be modified to reduce immunogenicity of the stem cell-derived β cell, and therefore exhibit improved survival, e.g., upon transplant. In some embodiments, the stem cell-derived β cells are modified to decrease or prevent an auto and/or allogeneic rejection of transplanted SC-islets. Modifications to the stem cell-derived β cells may decrease T cell activation, decrease NK cell activation, and increase SC-islet viability upon transplant.

In some embodiments, a stem cell, e.g., a pluripotent stem cell, an iPSC, a pancreatic progenitor, or a non-native β cell, is modified to overexpress or activate protective genes, e.g., to protect SC-islets or SC-beta cells from immune rejection. In some embodiments, a stem cell, e.g., an iPSC, a pancreatic progenitor, or a non-native β cell, is modified to reduce or eliminate expression of genes that contribute to an inflammatory response, e.g., that is directly or indirectly harmful to SC-islets or SC-beta cells. In some embodiments, a non-native β cell or precursor thereof is modified to perturbate the JAK/STAT type-II interferon (IFN) pathway. In some embodiments, the stem cell is modified to perturbate the IFN pathway prior to differentiating the stem cell into a non-native β cell, e.g., a stem cell-derived β cell. In some embodiments, IFNγ signaling mediators and downstream inflammatory components of the IFN pathway are targeted. In some embodiments, stem cell-derived β cells or precursors thereof are modified to decrease expression of one or more genes in the interferon (IFN) pathway. In some embodiments, the stem cell-derived β cells or precursors thereof are modified to reduce or eliminate expression of one or more genes listed in Table 1. In certain embodiments, the stem cell-derived β cells or precursors thereof are modified to reduce or eliminate expression of one or more genes selected from the group consisting of CXCL10 and STAT1. In one embodiment, the stem cell-derived β cell or precursor thereof is modified to reduce or eliminate expression of CXCL10. In one embodiment, the stem cell-derived β cell or precursor thereof is modified to reduce or eliminate expression of STAT1. In some embodiments, the stem cell-derived β cells or precursors thereof are further modified to reduce or eliminate expression of a gene selected from the group consisting of B2M, HLA-A, HLA-B, and TAP1.

TABLE 1

| | | | | |
|---|---|---|---|---|
| CXCL10 | URB1 | VPS54 | XCL2 | CCDC78 |
| ISG15 | PRAMEF18 | CCR3 | CXCL1 | CXCL5 |
| IRF1 | HSD17B12 | ATM | HLA-DRA | ODAD2 |
| HLA.A* | SIPA1L2 | TAP2 | BATF | PARD6B |
| HLA.B* | TMX3 | FSD2 | ZFAND1 | SLC25A2 |
| B2M* | GDAP1L1 | ABI1 | TOR2A | MVK |
| BST2 | PTGDS | ADAM21 | ZDHHC6 | ZNF613 |
| TAP1 | SCN1A | ANP32D | TBCD | IL11RA |
| IL32 | H2AC7 | C19orf71 | TRIL | H3C4 |
| HLA.C* | FGL1 | CBLC | FASTKD1 | GARNL3 |
| STAT1 | UVRAG | CRELD1 | CCDC80 | NCF1 |
| CXCL11 | RECQL | FAM187B | PLEKHA4 | LBHD1 |
| CXCL9 | CWC22 | FSD1 | KLF12 | ATXN1 |
| CD74 | MS4A4A | GABRA2 | ZNF14 | CHMP5 |
| TAP2 | KDM4C | LRRC2 | RER1 | SPATA31A7 |
| TNFSF10 | SLC39A6 | MMP15 | SIPA1L1 | COL21A1 |
| TAPBP | RAB3B | NDC1 | SLC22A11 | NFU1 |
| CD40 | SUPT16H | NOLC1 | SWAP70 | XKR4 |
| TAPBPL | GDE1 | PLEKHA8 | TMOD2 | |
| JAK1 | SNAP47 | QPCT | EEA1 | |
| JAK2 | IVL | RCE1 | HBG2 | |

In some embodiments, the stem cell-derived β cells or precursors thereof are modified to increase expression of one or more immunomodulatory factors or immune check point inhibitors. In some embodiments, the stem cell-derived β cells or precursors thereof are modified to increase expression of one or more genes listed in Table 2. In certain embodiments, the stem cell-derived β cells or precursors thereof are modified to increase expression of one or more genes selected from the group consisting of CD47, HLA-E, SOCS1, and PD-L1. In one embodiment, the stem cell-derived β cells or precursors thereof are modified to increase expression of CD47. In one embodiment, the stem cell-derived β cells or precursors thereof are modified to increase expression of PD-L1. In one embodiment, the stem cell-derived β cells or precursors thereof are modified to increase expression of SOCS1.

TABLE 2

| | | | |
|---|---|---|---|
| ABHD14A | LACTB2 | LRRC14B | FRMD8 |
| ARHGAP24 | NBPF9 | LRRC37A16P | TGFA |
| CDC37L1 | BOD1L1 | NCF1B | HLA-DMA |
| CHCHD10 | DBI | NTRK1 | DZIP1L |
| CHTOP | CDO1 | PIN4P1 | YBX2 |
| CYS1 | IGSF3 | PPP1R14BP5 | ZNF454 |
| ELP5 | XKR8 | PRR34-AS1 | YPEL4 |
| EOLA2 | WNK4 | RBFOX3 | FRS3 |
| ERAP1 | WDR61 | RPL22P10 | MOGAT3 |
| FAM241B | JAKMIP3 | RPL22P16 | HLA.E* |
| FOXC1 | IPO13 | RPL22P8 | SERPING1* |
| FOXJ3 | ZNF772 | RPL36AP46 | IL18BP |
| GIPC3 | MUC17 | RPS28P7 | IDO1 |
| GYPC | TRAPPC6A | RPS4XP2 | SOCS1 |
| HNRNPA1P10 | ZNF407 | RPS4XP3 | WARS |
| HNRNPA1P13 | SERPINB9* | RTL6 | CD274* |
| HNRNPA1P19 | PNKP | SDHAF1 | CD47* |
| HNRNPA1P47 | NFIA | SMIM29 | IL1RN |
| HNRNPA1P48 | TRIM51 | SNCAIP | SOCS3 |
| HNRNPA1P50 | CORO2A | SNRPA1P1 | TNFAIP3 |
| HNRNPA1P6 | C6orf136 | TNFRSF10A | PTPN2 |
| HNRNPA1P62 | GPR27 | TRPM2 | ZNF671 |
| HNRNPA1P63 | GABRA1 | TSPAN10 | PTPRA |
| HNRNPA1P7 | MND1 | UQCRHP2 | SEPTIN6 |
| KIRREL3 | CNTNAP2 | ZNF550 | NOTCH2NLA |
| PRDX1 | SLC9A2 | OCRL | LRP1 |
| THAP12 | SPATA21 | | |

In some embodiments modifying the expression of one or more genes in stem cell-derived β cells occurs using any gene editing tool known to those of skill in the art (e.g., TALENS, CRISPR, etc.). In some embodiments, the gene editing tool is delivered to the stem cells using a retrovirus (e.g., a lentivirus). In some embodiments, the one or more genes may be targeted using gene editing (e.g., CRISPR) to modulate expression of the one or more genes.

In some embodiments, the hypoimmunogenic stem cell-derived β cells or precursors thereof are maintained in culture by methods known by one of ordinary skill in the art, and in some embodiments, propagated prior to being converted into hypoimmunogenic stem cell-derived β cells by the methods as disclosed herein.

Further, at least one hypoimmunogenic stem cell-derived β cell or precursor thereof, e.g., pancreatic progenitor, can be from any mammalian species, with non-limiting examples including a murine, bovine, simian, porcine, equine, ovine, or human cell. For clarity and simplicity, the description of the methods herein refers to a mammalian hypoimmunogenic stem cell-derived β cell or precursor thereof but it should be understood that all of the methods described herein can be readily applied to other cell types of hypoimmunogenic stem cell-derived β cells or precursors thereof. In some embodiments, the hypoimmunogenic stem cell-derived β cells or precursors thereof are derived from a human individual.

In some embodiments, the hypoimmunogenic stem cell-derived β cells or precursors thereof are a substantially pure population of hypoimmunogenic stem cell-derived β cells or precursors thereof. In some embodiments, a population of hypoimmunogenic stem cell-derived β cells or precursors thereof comprises a mixture of pluripotent cells or differentiated cells (e.g., a mixture of SC-β cells, SC-α cells, SC-EC cells, and/or other differentiated cell types). In some embodiments, an SC-islet (e.g., a hypoimmunogenic SC-islet) comprises a mixture of pluripotent cells or differentiated cells (e.g., a mixture of SC-β cells, SC-α cells, SC-EC cells, and/or other differentiated cell types). In some embodiments, a population of hypoimmunogenic SC- cells or precursors thereof are substantially free or devoid of embryonic stem cells or pluripotent cells or iPS cells.

### Hypoimmunogenic Stem Cell-Derived β Cells

In some aspects of the disclosure, modified stem cell-derived β cells (e.g., hypoimmunogenic stem cell-derived β cells) are provided. The hypoimmunogenic stem cell-derived β cells disclosed herein share many distinguishing features of native pancreatic cells but are different in certain aspects. In some embodiments, the stem cell-derived β cells are non-native, i.e., non-naturally occurring, non-endogenous cells. As used herein, “non-native” means that the modified stem cell-derived β cells are markedly different in certain aspects from cells which exist in nature, i.e., native cells. It should be appreciated, however, that these marked differences may result in the modified stem cell-derived β cells exhibiting certain differences, but the modified stem cell-derived β cells may still behave in a similar manner to native cells with certain functions altered (e.g., improved) compared to the native cells.

Stem cell-derived β cells are differentiated in vitro from any starting cell as the invention is not intended to be limited by the starting cell from which the stem cell-derived β cells are derived. Exemplary starting cells include, without limitation, endocrine cells or any precursor thereof such as a NKX6-1+ pancreatic progenitor cell, a Pdx1+ pancreatic progenitor cell, and a pluripotent stem cell, an embryonic stem cell, and induced pluripotent stem cell. In some embodiments, the stem cell-derived cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), a transdifferentiated cell. In some embodiments, the stem cell-derived β cells disclosed herein can be differentiated in vitro from an endocrine cell or a precursor thereof. In some embodiments, the stem cell-derived 3 cell is differentiated in vitro from a precursor selected from the group consisting of a NKX6-1+ pancreatic progenitor cell, a Pdx1+ pancreatic progenitor cell, and a pluripotent stem cell. In some embodiments, the pluripotent stem cell is selected from the group consisting of an embryonic stem cell and induced pluripotent stem cell. In some embodiments, the stem cell-derived 3 cell or the pluripotent stem cell from which the stem cell-derived β cell is derived is human. In some embodiments, the stem cell-derived 3 cell is human.

In some embodiments, the stem-cell derived β cells are modified to overexpress or activate protective genes. In some embodiments, the stem cell-derived β cells are modified to eliminate genes that contribute to an inflammatory response. In certain embodiments, the stem cell-derived β cells comprise a perturbation in the IFN pathway, e.g., a modification to an IFNγ signaling mediator and/or a downstream inflammatory component. Stem cell-derived β cells comprising a perturbation in the IFN pathway may be hypoimmunogenic stem cell-derived β cells.

Hypoimmunogenic stem cell-derived β cells may exhibit decreased risk of auto and/or allogeneic rejection upon transplant. In addition, hypoimmunogenic stem cell-derived β cells may exhibit decreased T cell activation, decreased NK cell activation, and/or increased viability, e.g., upon transplant. In some embodiments, hypoimmunogenic stem cell-derived β cells are able to evade the immune system upon transplant. In some embodiments, hypoimmunogenic stem cell-derived β cells exhibit improved cell survival under immune rejection.

In some embodiments, a hypoimmunogenic stem cell-derived β cell comprises reduced or eliminated expression of one or more genes selected from Table 1. In certain embodiments, the hypoimmunogenic stem cell-derived β cell comprises reduced expression of one or more genes selected from the group consisting of CXCL10 and STAT1. In one embodiment, the hypoimmunogenic stem cell-derived R cell does not express CXCL10. In one embodiment, the hypoimmunogenic stem cell-derived β cell does not express STAT1. In some embodiments, the hypoimmunogenic stem cell-derived β cell further comprises reduced expression of one or more genes selected from the group consisting of B2M, HLA-A, HLA-B, and TAP1. In some embodiments, the hypoimmunogenic stem cell-derived β cell comprises increased or activated expression of one or more genes selected from Table 2. In certain embodiments, the hypoimmunogenic stem cell-derived β cell comprises increased or activated expression of one or more genes selected from the group consisting of CD47, PD-L1, SOCS1, and HLA-E. In one embodiment, the hypoimmunogenic stem cell-derived β cell comprises increased expression of CD47. In one embodiment, the hypoimmunogenic stem cell-derived β cell comprises increased expression of PD-L1. In one embodiment, the hypoimmunogenic stem cell-derived β cell comprises increased expression of SOCS1.

In some embodiments, the hypoimmunogenic stem cell-derived β cell is not genetically modified. In some embodiments, the hypoimmunogenic stem cell-derived β cell obtains the features it shares in common with native cells in the absence of a genetic modification of cells. In some embodiments, the hypoimmunogenic stem cell-derived β cell is genetically modified.

In some aspects, the disclosure provides a cell line comprising a hypoimmunogenic stem cell-derived β cell described herein. In some aspects, the disclosure provides an SC-islet comprising hypoimmunogenic stem cell-derived R cells described herein in combination with SC-α cells, SC-δ cells, and/or SC-EC cells.

In some embodiments, the cells described herein, e.g., a population of hypoimmunogenic stem cell-derived β cells are transplantable, e.g., a population of hypoimmunogenic stem cell-derived β cells can be administered to a subject. In some embodiments, an SC-islet comprising hypoimmunogenic stem cell-derived β cells is transplantable, e.g., an SC-islet can be administered to a subject. In some embodiments, the subject who is administered a population of hypoimmunogenic stem cell-derived β cells is the same subject from whom a pluripotent stem cell used to differentiate into a hypoimmunogenic stem cell-derived β cell was obtained (e.g., for autologous cell therapy). In some embodiments, the subject is a different subject. In some embodiments, a subject is suffering from an intestinal disorder such as intestinal inflammation or is a normal subject. For example, the cells for transplantation (e.g., a composition comprising a population of hypoimmunogenic stem cell-derived β cells or an SC-islet comprising a population of hypoimmunogenic stem cell-derived β cells) can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of hypoimmunogenic stem cell-derived cells (e.g., hypoimmunogenic pancreatic stem cell-derived cells, such as SC-β cells and/or SC-α cells) can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

For administration to a subject, a cell population produced by the methods as disclosed herein, e.g., a population of hypoimmunogenic stem cell-derived cells (e.g., hypoimmunogenic stem cell-derived β cells) or an SC-islet comprising hypoimmunogenic stem cell-derived cells can be administered to a subject, for example in pharmaceutically acceptable compositions. These pharmaceutically acceptable compositions comprise a therapeutically effective amount of a population of hypoimmunogenic stem cell-derived cells as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., hypoimmunogenic stem cell-derived cells, or a composition comprising hypoimmunogenic stem cell-derived cells of the present invention which is effective for producing some desired therapeutic effect in at least a sub-population of cells in an animal at a reasonable benefit/risk ratio applicable to any medical treatment. For example, an amount of a population of hypoimmunogenic stem cell-derived cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment,” “prevention,” or “amelioration” of a disease or disorder is meant delaying or preventing the onset of such a disease or disorder, reversing, alleviating, ameliorating, inhibiting, slowing down or stopping the progression, aggravation or deterioration of the progression or severity of a condition associated with such a disease or disorder. In one embodiment, the symptoms of a disease or disorder are alleviated by at least 5%, at least 10%, at least 20%, at least 30%, at least 40%, or at least 50%.

Treatment of diabetes is determined by standard medical methods. A goal of diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patient, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having diabetes (e.g., Type 1 or Type 2), one or more complications related to diabetes, or a pre-diabetic condition, and optionally, but need not have already undergone treatment for diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes, one or more complications related to diabetes, or a pre-diabetic condition. For example, a subject can be one who exhibits one or more risk factors for diabetes, complications related to diabetes, or a pre-diabetic condition, or a subject who does not exhibit diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

As used herein, the phrase “subject in need of pancreatic hypoimmunogenic stem cell-derived cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of hypoimmunogenic pancreatic stem cell-derived cells can be identified using any method used for diagnosis of diabetes. For example, Type 1 diabetes can be diagnosed using a glycosylated hemoglobin (A1C) test, a random blood glucose test and/or a fasting blood glucose test. Parameters for diagnosis of diabetes are known in the art and available to skilled artisan without much effort.

In some embodiments, the methods of the invention further comprise selecting a subject identified as being in need of additional pancreatic hypoimmunogenic stem cell-derived cells. A subject in need a population of pancreatic hypoimmunogenic stem cell-derived cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, a composition comprising a population of hypoimmunogenic stem cell-derived cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

A composition comprising hypoimmunogenic stem cell-derived cells can be administrated to the subject at the same time or at different times as the administration of a pharmaceutically active agent or composition comprising the same. When administrated at different times, the compositions comprising a population of hypoimmunogenic stem cell-derived cells and/or pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When a composition comprising a population of hypoimmunogenic stem cell-derived cells and a composition comprising a pharmaceutically active agent are administered in different pharmaceutical compositions, routes of administration can be different. In some embodiments, a subject is administered a composition comprising hypoimmunogenic stem cell-derived cells. In other embodiments, a subject is administered a composition comprising a pharmaceutically active agent. In another embodiment, a subject is administered a composition comprising a population of hypoimmunogenic stem cell-derived cells mixed with a pharmaceutically active agent. In another embodiment, a subject is administered a composition comprising a population of hypoimmunogenic stem cell-derived cells and a composition comprising a pharmaceutically active agent, where administration is substantially at the same time, or subsequent to each other.

Toxicity and therapeutic efficacy of administration of a composition comprising a population of stem cell-derived cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of hypoimmunogenic stem cell-derived cells that exhibit large therapeutic indices are preferred.

The amount of a composition comprising a population of hypoimmunogenic stem cell-derived cells can be tested using several well-established animal models.

The non-obese diabetic (NOD) mouse carries a genetic defect that results in insulitis showing at several weeks of age (Yoshida et al., Rev. Immunogenet. 2:140, 2000). 60-90% of the females develop overt diabetes by 20-30 weeks. The immune-related pathology appears to be similar to that in human Type I diabetes. Other models of Type I diabetes are mice with transgene and knockout mutations (Wong et al., Immunol. Rev. 169:93, 1999). A rat model for spontaneous Type I diabetes was recently reported by Lenzen et al. (Diabetologia 44:1189, 2001). Hyperglycemia can also be induced in mice (>500 mg glucose/dL) by way of a single intraperitoneal injection of streptozotocin (Soria et al., Diabetes 49:157, 2000), or by sequential low doses of streptozotocin (Ito et al., Environ. Toxicol. Pharmacol. 9:71, 2001). To test the efficacy of implanted islet cells, the mice are monitored for return of glucose to normal levels (<200 mg/dL).

Larger animals provide a good model for following the sequelae of chronic hyperglycemia. Dogs can be rendered insulin-dependent by removing the pancreas (J. Endocrinol. 158:49, 2001), or by feeding galactose (Kador et al., Arch. Opthalmol. 113:352, 1995). There is also an inherited model for Type I diabetes in keeshond dogs (Am. J. Pathol. 105:194, 1981). Early work with a dog model (Banting et al., Can. Med. Assoc. J. 22:141, 1922) resulted in a couple of Canadians making a long ocean journey to Stockholm in February of 1925.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of hypoimmunogenic stem cell-derived cells can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the stem cell-derived cells. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of hypoimmunogenic stem cell-derived cells as disclosed herein. In one embodiment of the invention, an isolated population of hypoimmunogenic stem cell-derived cells as disclosed herein may be used for the production of a pharmaceutical composition, for use in transplantation into subjects in need of treatment, e.g., a subject that has, or is at risk of developing diabetes, for example but not limited to subjects with congenital and acquired diabetes. In one embodiment, an isolated population of hypoimmunogenic stem cell-derived cells may be genetically modified. In another aspect, the subject may have or be at risk of diabetes and/or metabolic disorder. In some embodiments, an isolated population of hypoimmunogenic stem cell-derived cells as disclosed herein may be autologous and/or allogeneic. In some embodiments, the subject is a mammal, and in other embodiments the mammal is a human.

The use of an isolated population of hypoimmunogenic stem cell derived cells as disclosed herein provides advantages over existing methods because the population of hypoimmunogenic stem cell-derived cells can be differentiated from endocrine progenitor cells or precursors thereof derived from stem cells, e.g., iPS cells obtained or harvested from the subject administered an isolated population of hypoimmunogenic stem cell-derived cells. This is highly advantageous as it provides a renewable source of hypoimmunogenic stem cell-derived cells which can be differentiated from stem cells to endocrine progenitor cells by methods commonly known by one of ordinary skill in the art, and then further differentiated by the methods described herein to pancreatic j-like cells, for transplantation into a subject, in particular an SC-islet comprising hypoimmunogenic pancreatic j-like cells that do not have the risks and limitations of cells derived from other systems.

One embodiment of the invention relates to a method of treating diabetes or a metabolic disorder in a subject comprising administering an effective amount of a composition comprising a population of hypoimmunogenic stem cell-derived cells (e.g., hypoimmunogenic pancreatic stem cell-derived cells) as disclosed herein to a subject with diabetes and/or a metabolic disorder. In a further embodiment, the invention provides a method for treating diabetes, comprising administering a composition comprising a population of hypoimmunogenic stem cell-derived cells as disclosed herein to a subject that has, or has an increased risk of developing diabetes.

In one embodiment of the above methods, the subject is a human and a population of hypoimmunogenic stem cell-derived cells as disclosed herein are human cells. In some embodiments, the invention contemplates that a population of hypoimmunogenic stem cell-derived cells as disclosed herein are administered directly to the pancreas of a subject or is administered systemically. In some embodiments, a population of hypoimmunogenic stem cell-derived cells as disclosed herein can be administered to any suitable location in the subject, for example in a capsule in the blood vessel or the liver.

The present invention is also directed to a method of treating a subject with diabetes or a metabolic disorder which occurs as a consequence of genetic defect, physical injury, environmental insult or conditioning, bad health, obesity and other diabetes risk factors commonly known by a person of ordinary skill in the art. Efficacy of treatment of a subject administered a composition comprising a population of hypoimmunogenic stem cell-derived cells (e.g., hypoimmunogenic pancreatic stem cell-derived cells) can be monitored by clinically accepted criteria and tests, which include for example, (i) Glycated hemoglobin (A1C) test, which indicates a subjects average blood sugar level for the past two to three months, by measuring the percentage of blood sugar attached to hemoglobin, the oxygen-carrying protein in red blood cells. The higher your blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of hypoimmunogenic stem cell-derived cells (e.g., hypoimmunogenic pancreatic stem cell-derived cells) as disclosed herein to a subject in need thereof is associated with improved exercise tolerance or other quality of life measures, and decreased mortality. The effects of cellular therapy with a population of hypoimmunogenic stem cell-derived cells can be evident over the course of days to weeks after the procedure. However, beneficial effects may be observed as early as several hours after the procedure and may persist for several years. In some embodiments, the effects of cellular therapy with a population of hypoimmunogenic stem cell-derived cells occurs within two weeks after the procedure.

In some embodiments, a population of hypoimmunogenic stem cell-derived cells (e.g., hypoimmunogenic pancreatic stem cell-derived cells) as disclosed herein may be used for tissue reconstitution or regeneration in a human patient or other subject in need of such treatment. In some embodiments compositions of populations of hypoimmunogenic stem cell-derived cells can be administered in a manner that permits them to graft or migrate to the intended tissue site and reconstitute or regenerate the functionally deficient area. Special devices are available that are adapted for administering cells capable of reconstituting a population of hypoimmunogenic pancreatic cells (e.g., β cells) in the pancreas or at an alternative desired location. Accordingly, the hypoimmunogenic stem cell-derived cells may be administered to a recipient subject's pancreas by injection or administered by intramuscular injection.

### EXEMPLIFICATION

One hundred years ago, the first type 1 diabetes (T1D) diabetic patient was treated in Toronto with a “pancreatic extract”, part of a series of experiments that later led to the discovery of insulin (Banting et al., 1922). Since then, the scientific basis for T1D has been described as an autoimmune attack of pancreatic insulin producing β-cells. However, despite technological advances, such as insulin pumps and continuous glucose monitoring devices (Kovatchev, 2019), exogenous insulin administration remains the only option to regulate blood glucose levels. Nevertheless, in the last few decades β-cells replacement strategies emerged as a new hope for patients, beginning when cadaveric islet transplantations demonstrated insulin independence (Shapiro et al., 2000) and followed by the exploration of human pluripotent stem cells (hPSC) as an unlimited source for beta-cell differentiation and replacement.

Despite these advances, a major challenge remains for protecting SC-islets against an allogeneic response and recurring β-cell destruction due to autoimmunity. The use of immunosuppressor drugs can lead to complications and may lead to graft impairment in the long term (Lehmann et al., 2008). Encapsulation methods and cell capturing devices are useful for immune protection and possible graft extraction, but still exhibit issues with keeping oxygen and nutrients in the graft and insulin released from the graft, while avoiding difficulties like fibrotic foreign-body response and hypoxia (Kharbikar et al., 2021) (Alagpulinsa et al., 2019).

Although modulation of the immune system by regulatory T cells has been suggested (Raffin et al., 2020), great effort has been dedicated recently to genetically modifying hPSC4s to create donor universal lines for cell replacement therapies that could be transplanted ‘naked’ without the use of immunosuppressor drugs. Strategies include β-2-microglobulin (B2M) or HLA-1/J depletions (Castro-Gutierrez et al., 2021; Deuse et al., 2019; Han et al., 2019; Parent et al., 2021; Wang et al., 2015) to prevent allo- and auto-antigen presentation of donor cells and expression of immune check point inhibitors like PD-L1 (Castro-Gutierrez et al., 2021; Yoshihara et al., 2020). Other approaches like expression of CD47 (Deuse et al., 2021; Deuse et al., 2019) and HLA-E (Gornalusse et al., 2017) mean to confront NK killing when HLA-A,-B,-C are removed directly or indirectly (e.g. by B2M knockout), or by removing only HLA-A and HLA-B in iPSC but retaining one HLA-C alle, requiring only a small number of compatible lines to cover most of recipient populations across the world (Xu et al., 2019). These strategies are highly promising, but they all derive from previous knowledge based on numerous studies in cancer, pathogens, or mother-fetus interactions. There is limited understanding of newly found endocrine cell-related targets for immune modulation against β-cell attack (Cai et al., 2020; Wei et al., 2018).

### Results

Single Cell Transcriptional Analysis Reveals ‘Alarm’ Genes that Drive Immunogenicity of SC-Islets and Leads to T-Cell Activation and Killing

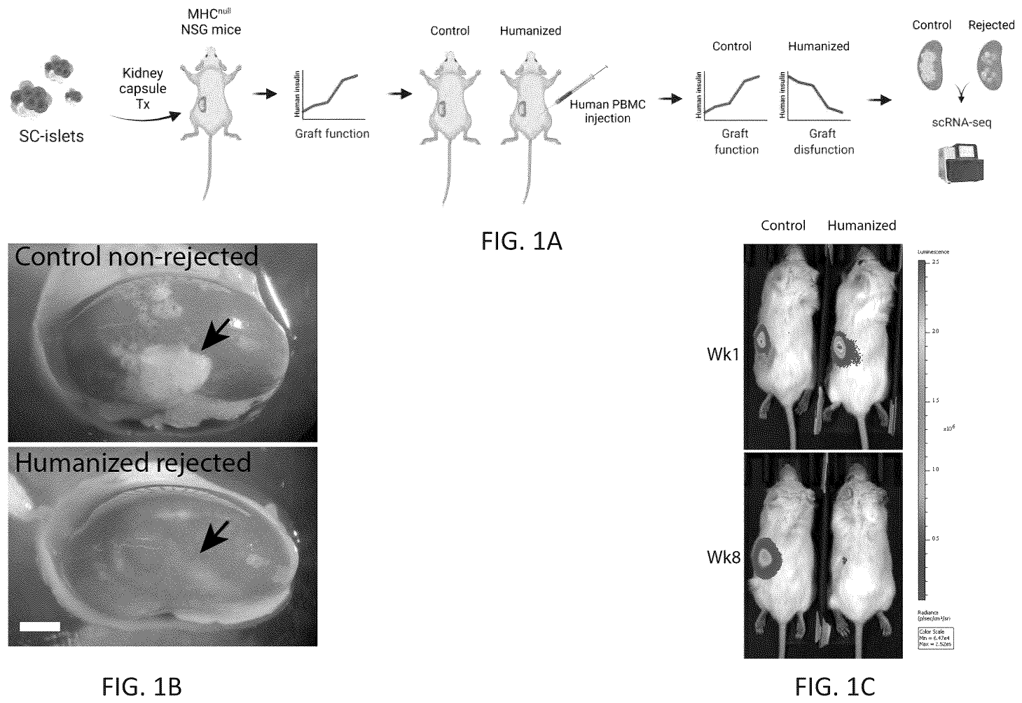

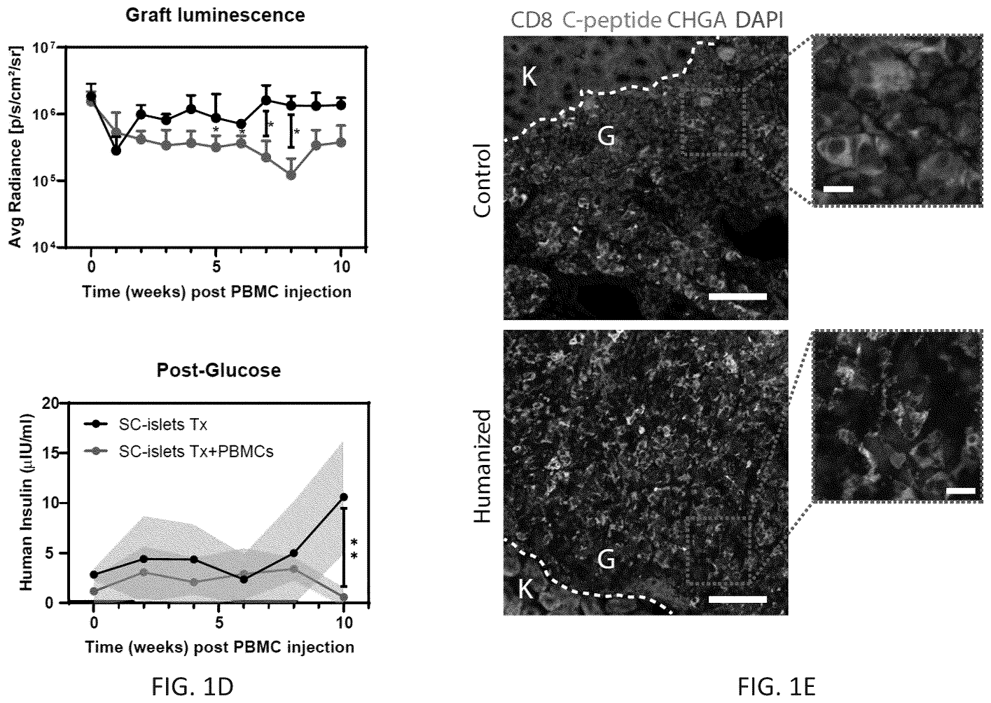

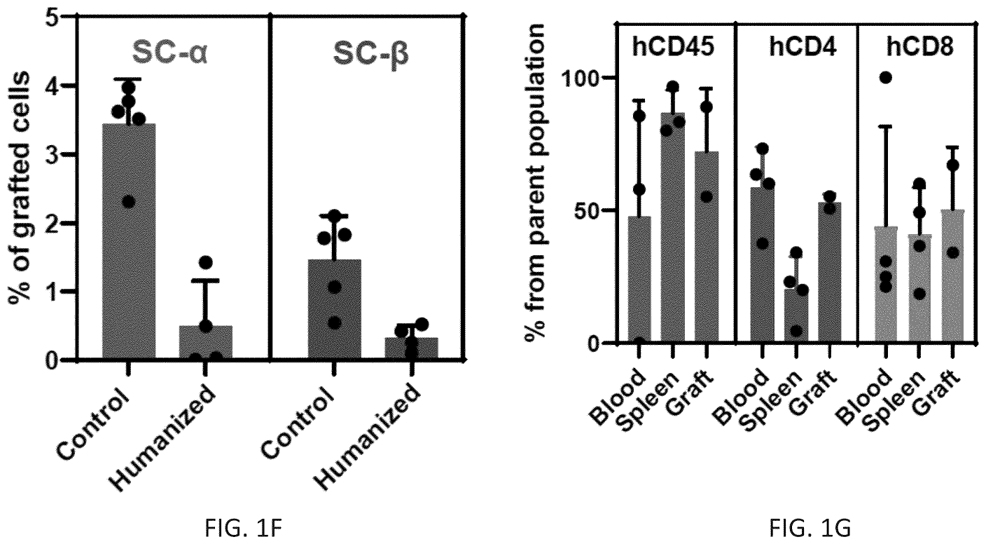

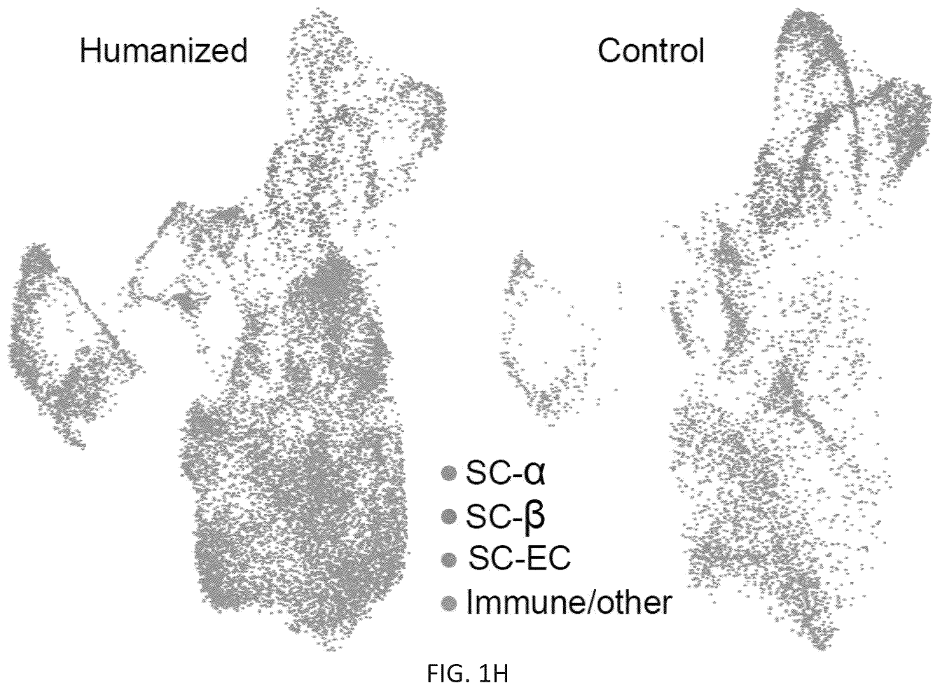

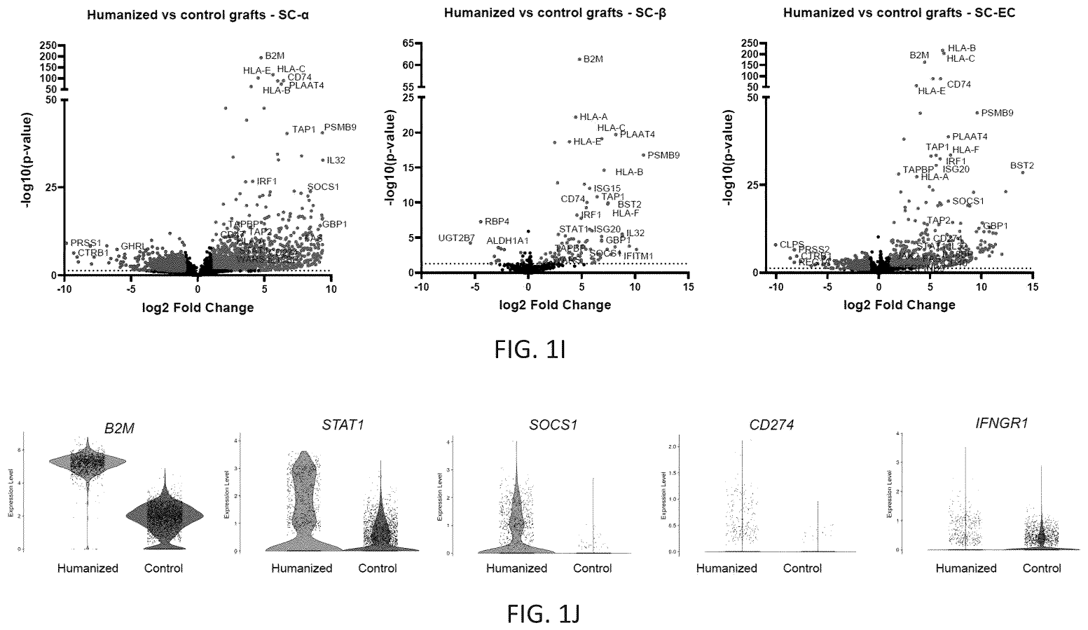

In order to study immune responses in the context of human allogeneic graft rejection, the Hu-PBL-NSG-MHC^{null} platform was chosen (Brehm et al., 2019). NOD-scid IL-2 receptor subunit γ (IL2rg)^{null} (NSG) immunocompromised mice that lack murine MHC class I and II, were transplanted (under kidney capsule) with SC-islets (HLA-A2 positive), followed by human PBMC injection (n=6 mice termed ‘humanized’) from healthy unmatched donors (HLA-A2 negative) while leaving half of the cohort (n=6 mice) without injection as control (FIG. 1A). The lack of murine MHC allowed for monitoring the function grafts for prolonged durations without the risk of xenogeneic graft vs host disease (GVHD). Graft function failure due to rejection was determined either by bioluminescence emission from transplanted GAPDH-luciferase SC-islets (Gerace et al., 2021) (FIGS. 1C-1D), or by human insulin detection from mouse blood (FIG. 1D), 30 min after glucose injection. Reduction in graft size (FIGS. 1B-1C) and the loss of function by glucose sensitivity, was attributed mainly to T-cells that retained in mouse tissues (FIG. 1G) for the entire experiment. Furthermore, CD8 cytotoxic T-cells (CTLs) can be clearly seen infiltrating the SC-islet grafts (FIG. 1E) of humanized mice (control mice shows no T-cells, only autofluorescence from red blood cells) in early time points and contacting endocrine (chromogranin A positive) and SC-β cells (C-peptide positive). SC-islets are comprised with several pancreatic hormone producing cell populations, including glucagon expressing SC-α cells and insulin expressing SC-β cells. At 10-weeks post PBMC injections, it was observed that both SC-α and SC-β numbers are reduced in humanized mouse grafts compared to controls (FIG. 1F), typical to an unselective allogeneic response.

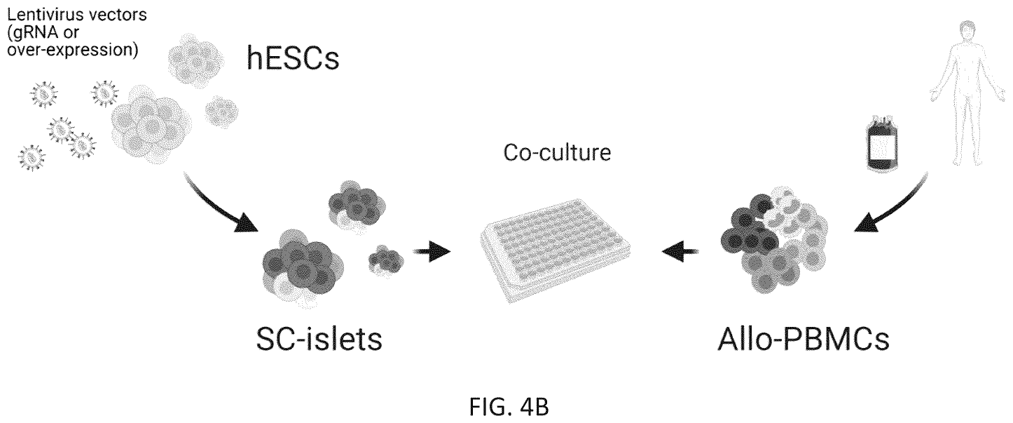

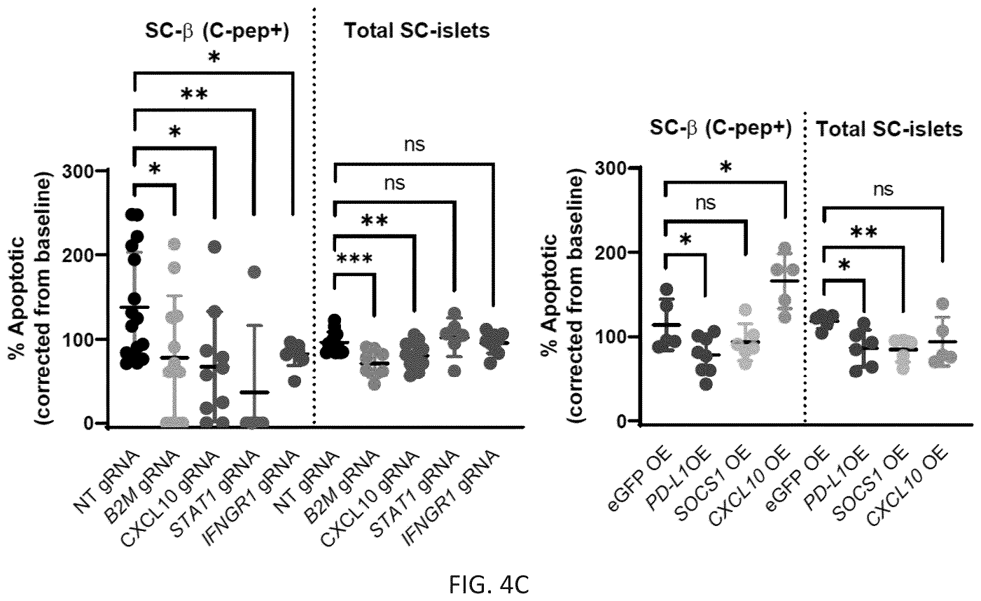

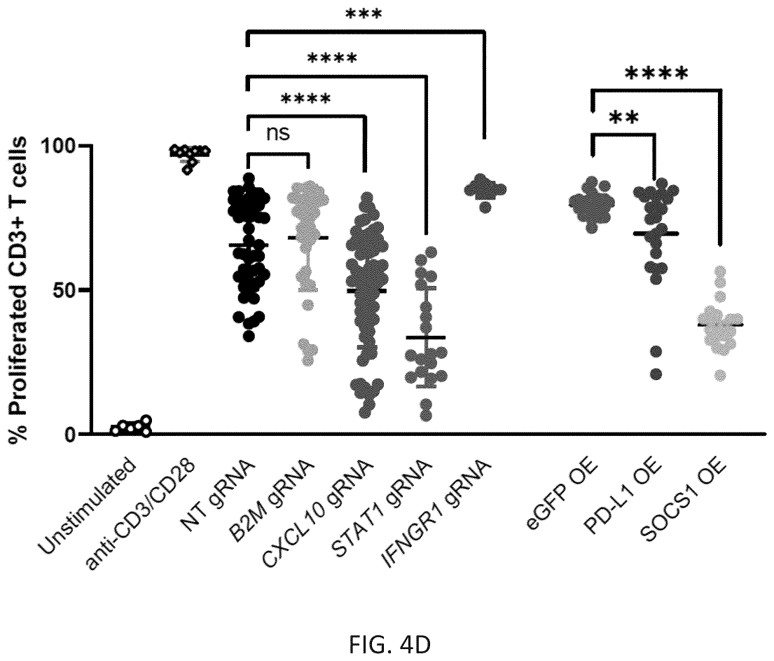

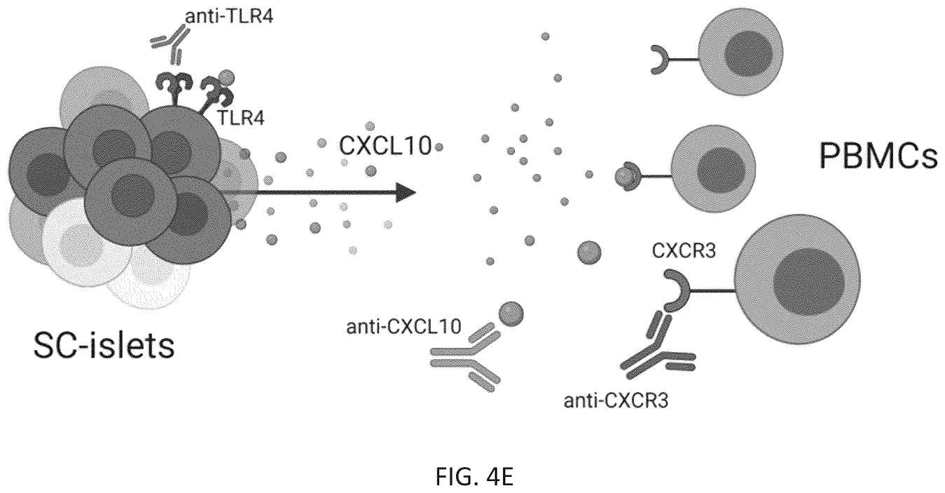

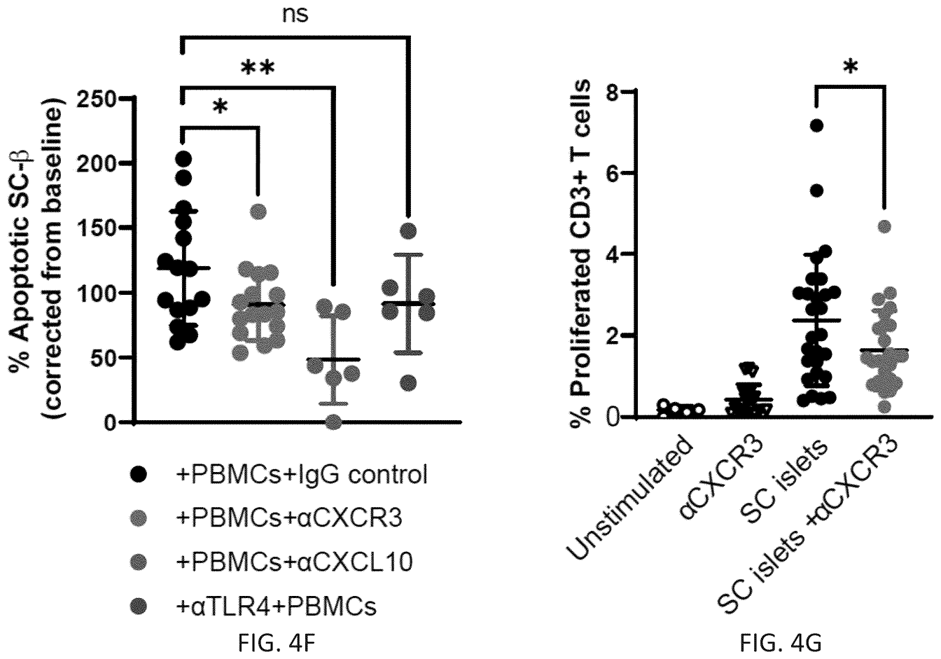

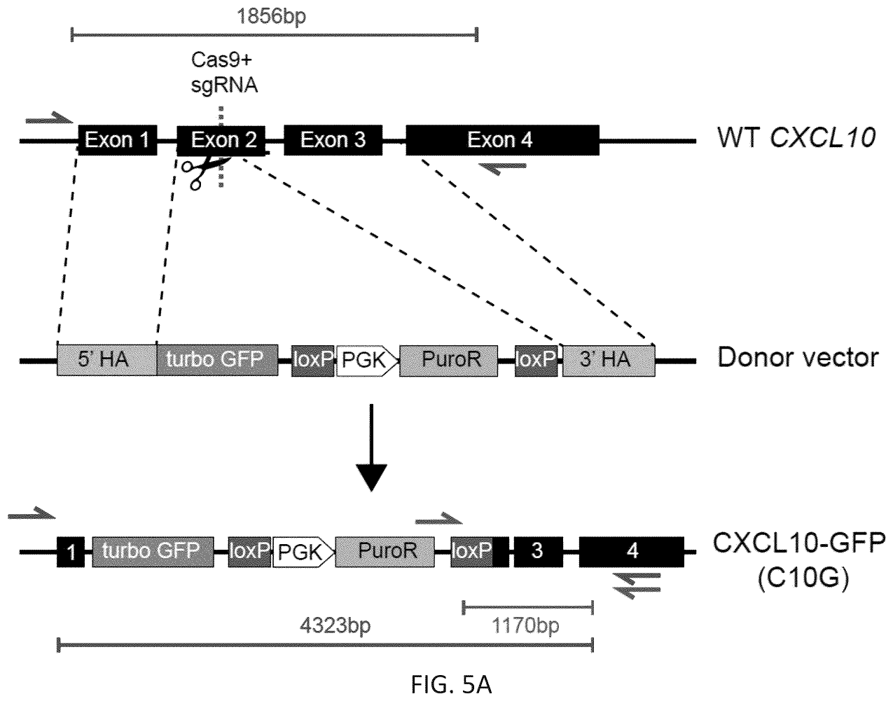

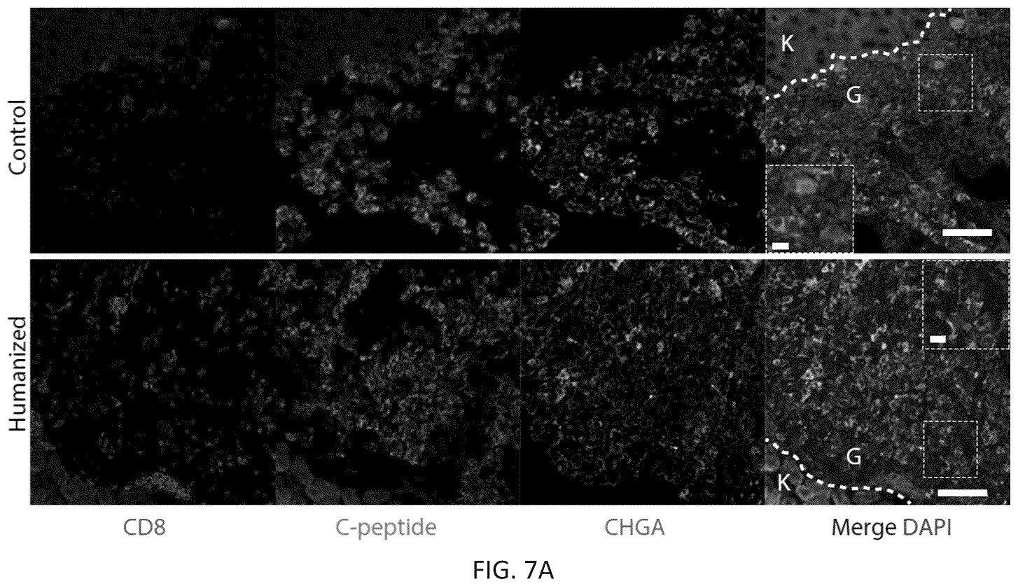

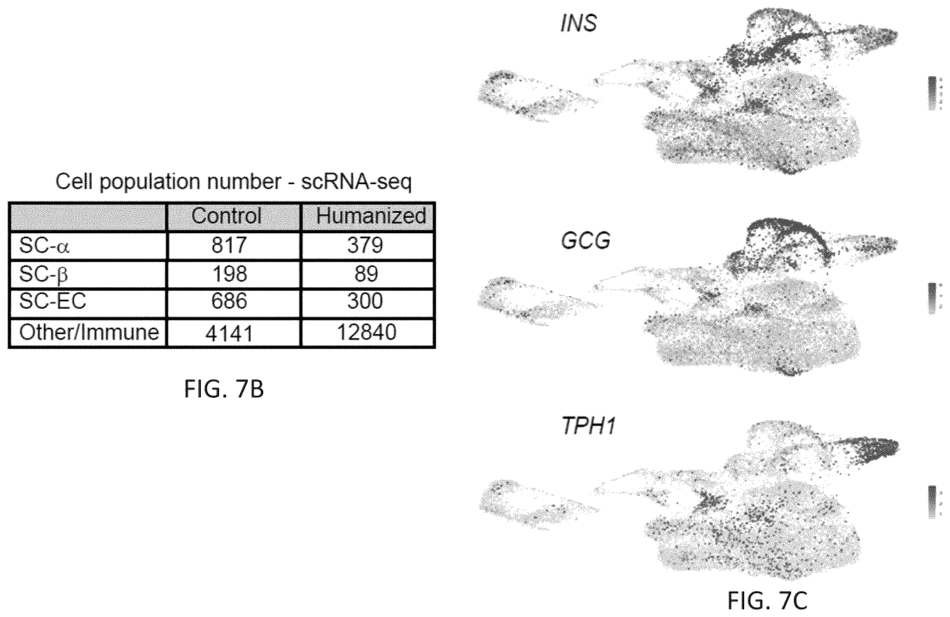

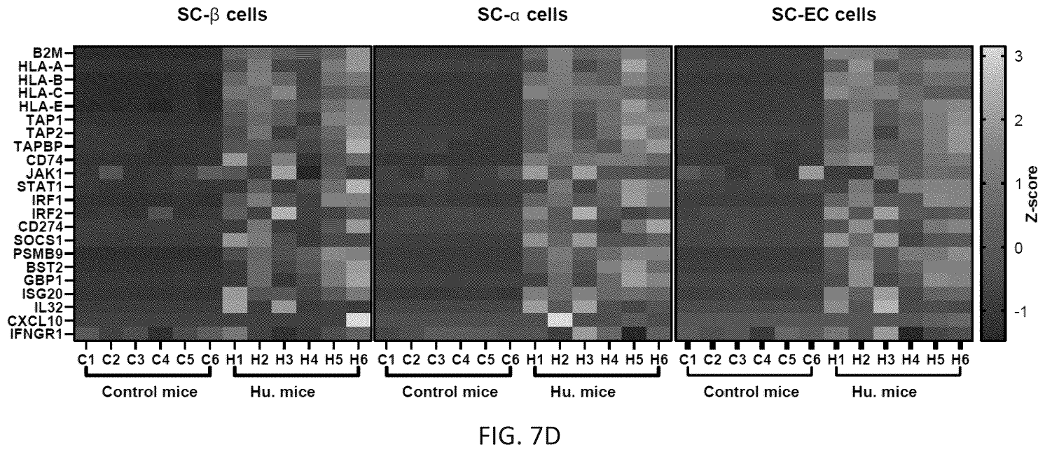

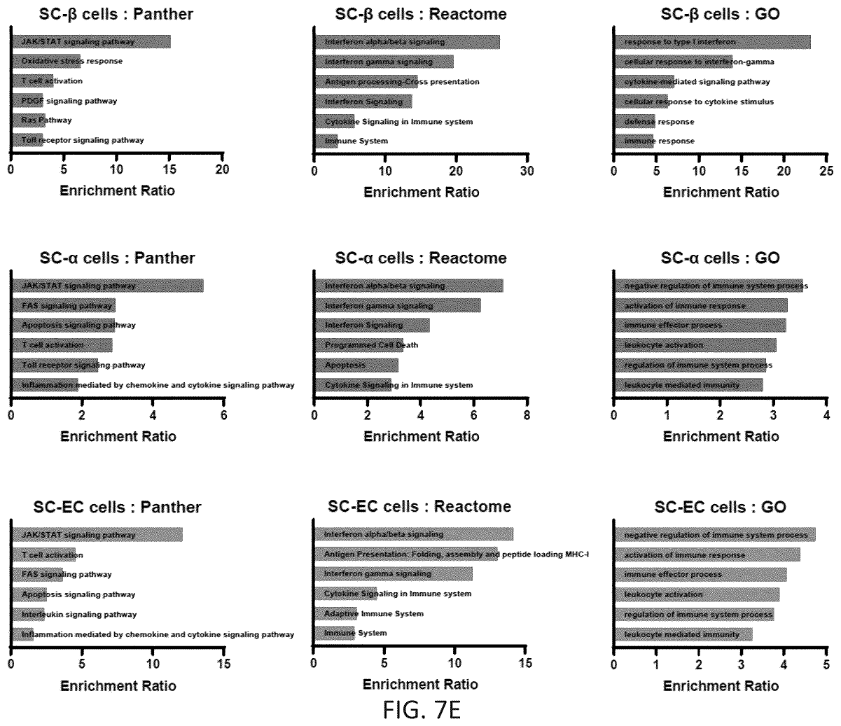

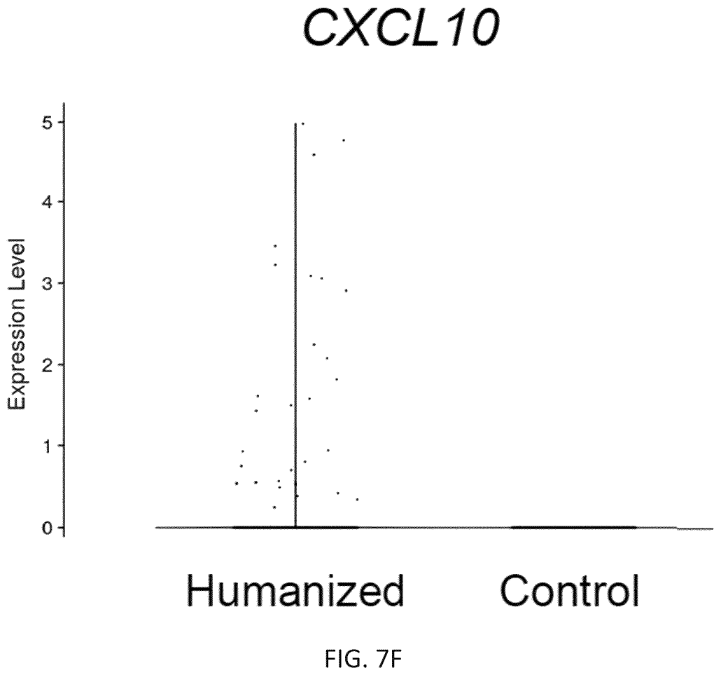

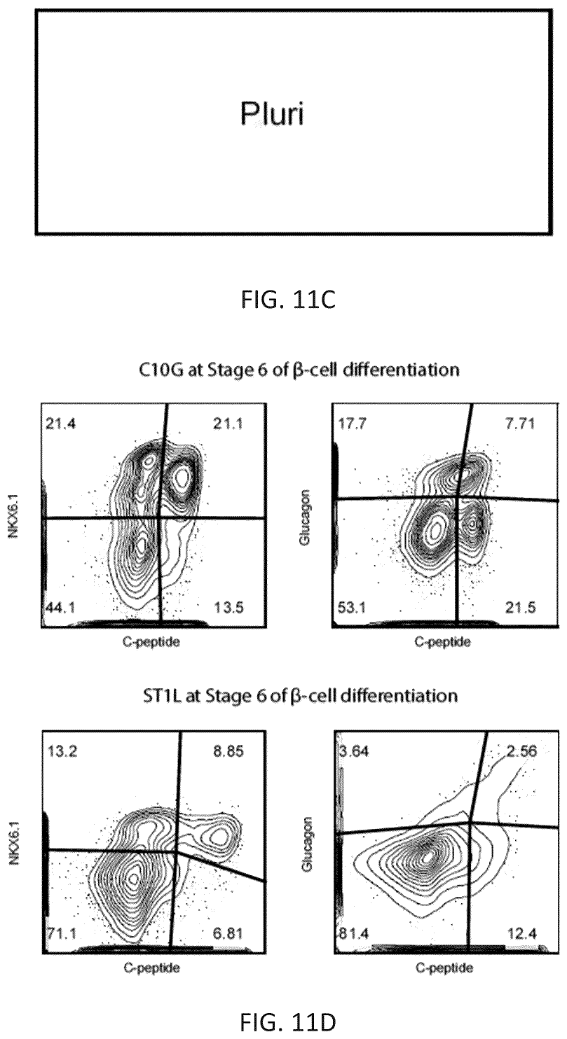

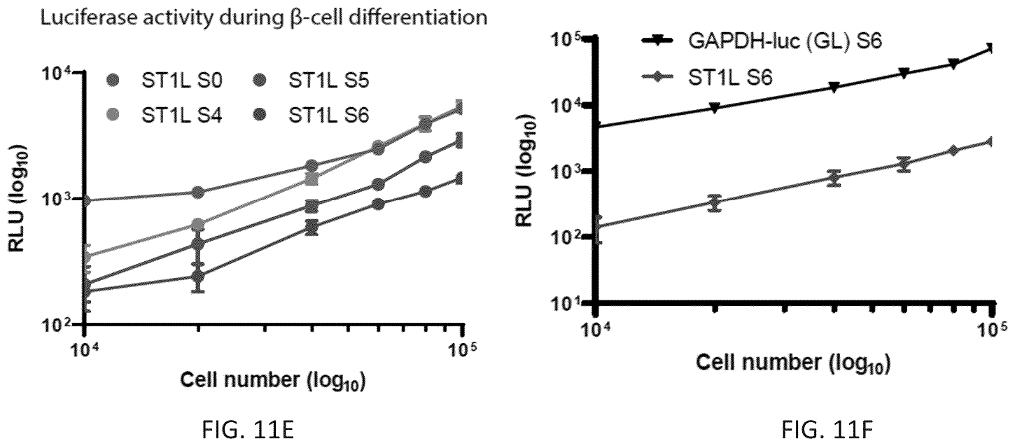

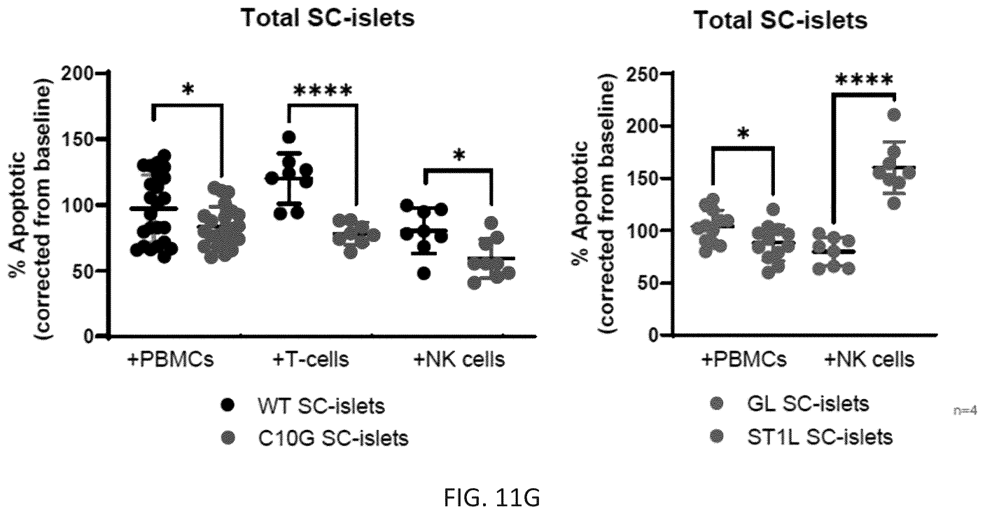

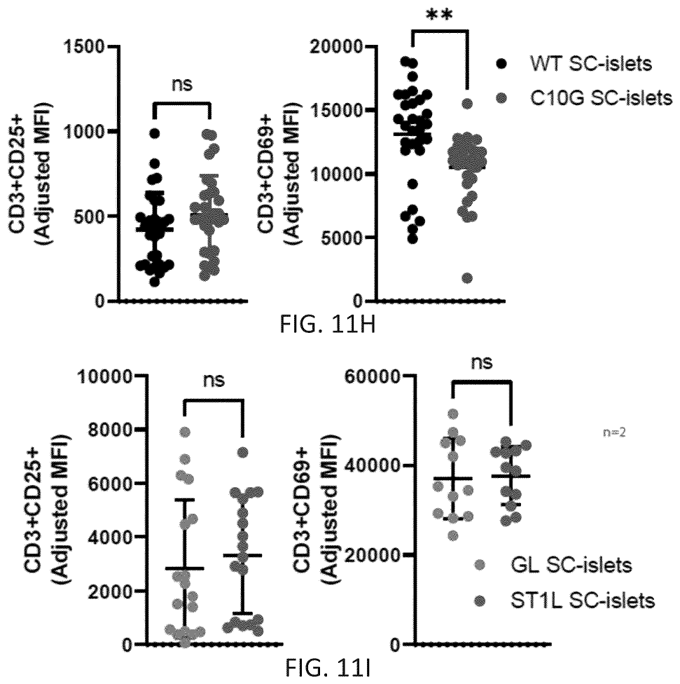

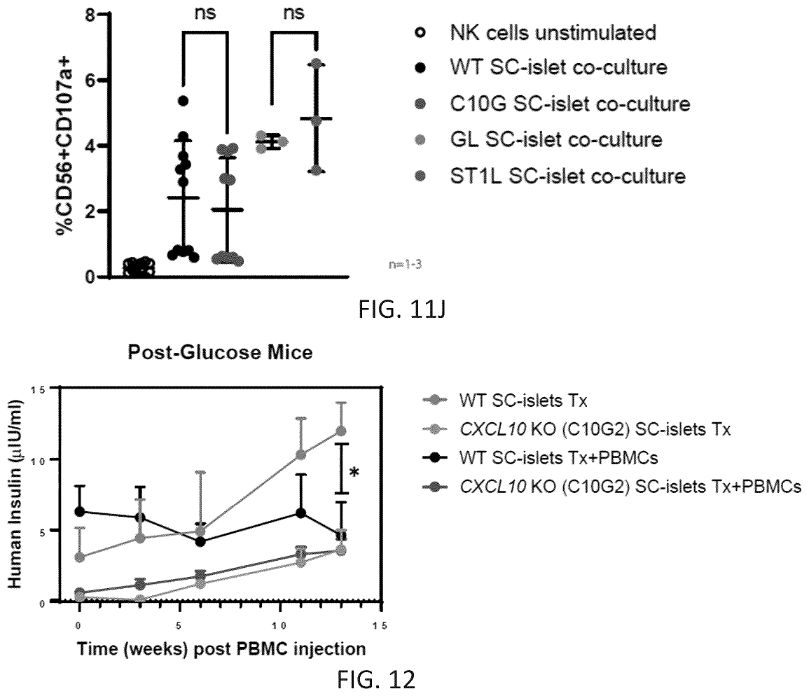

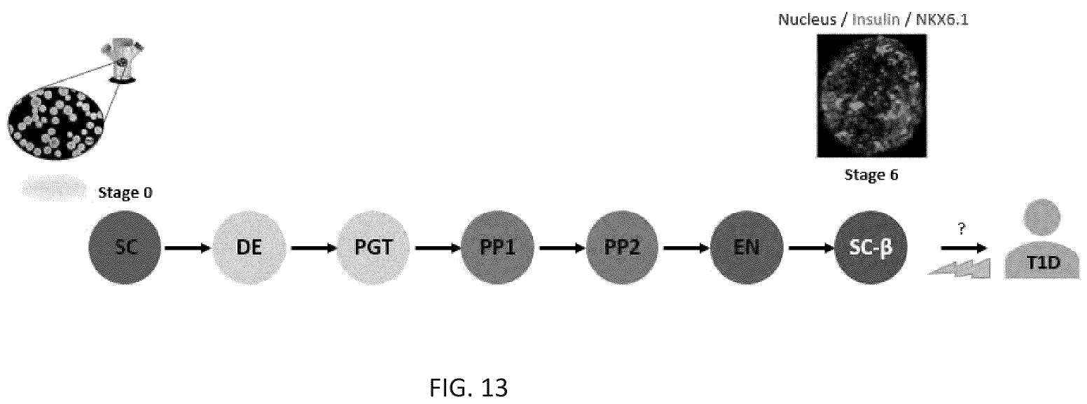

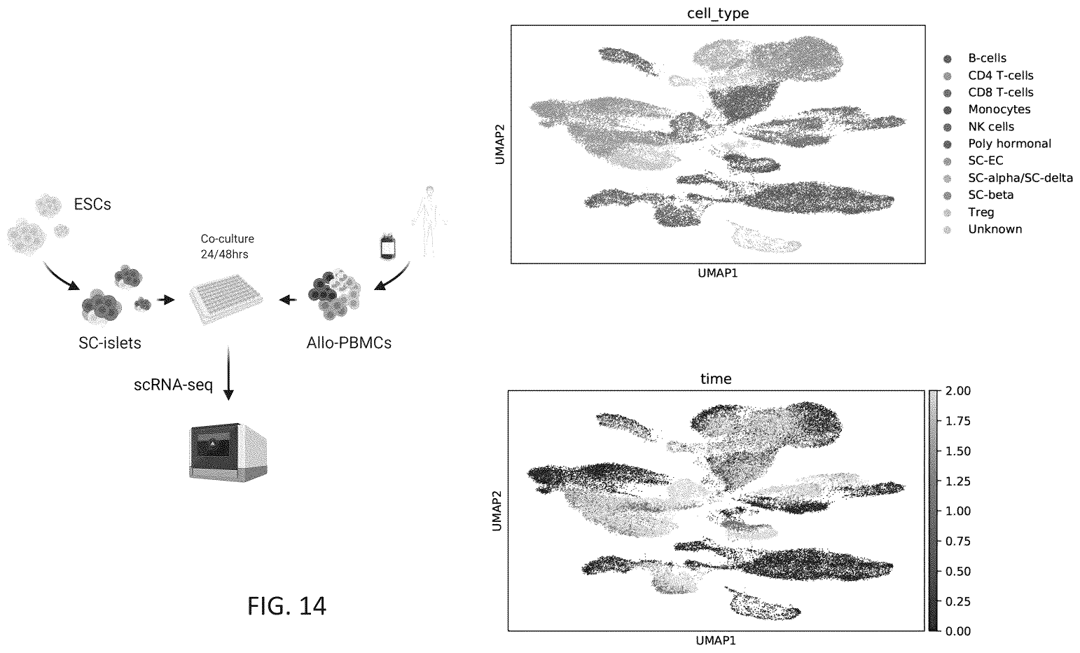

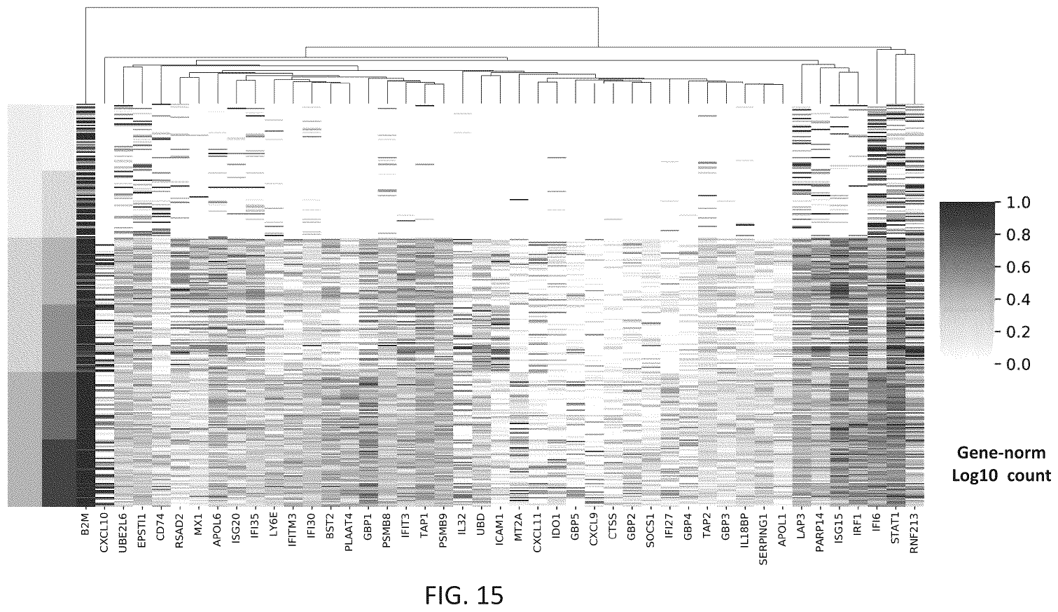

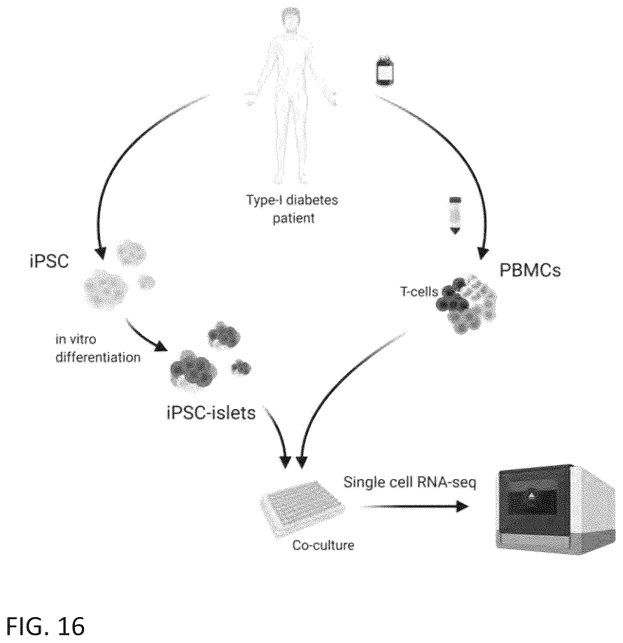

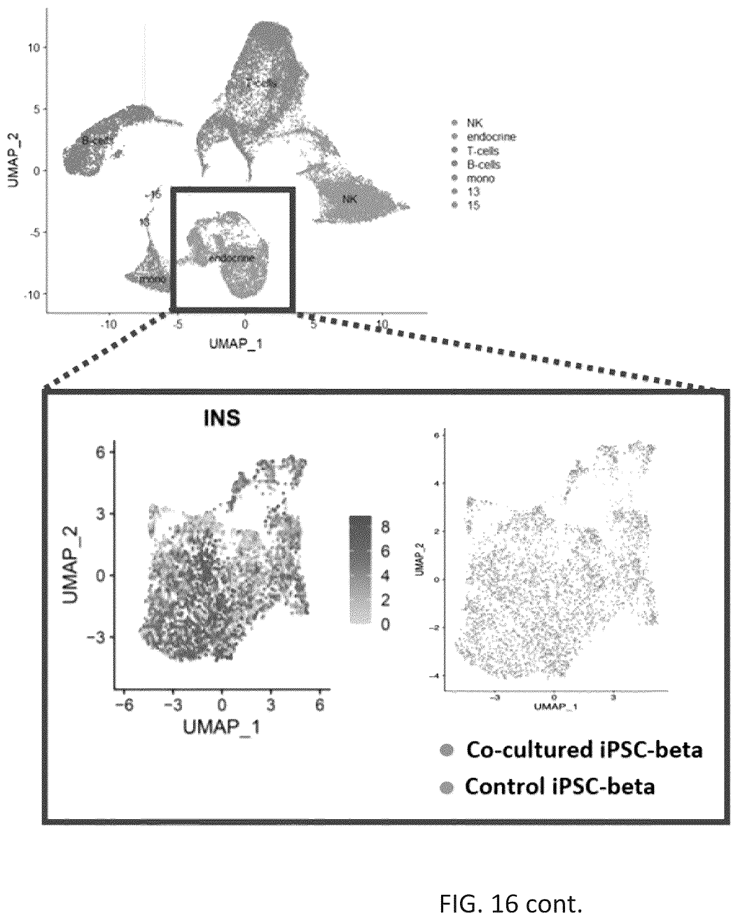

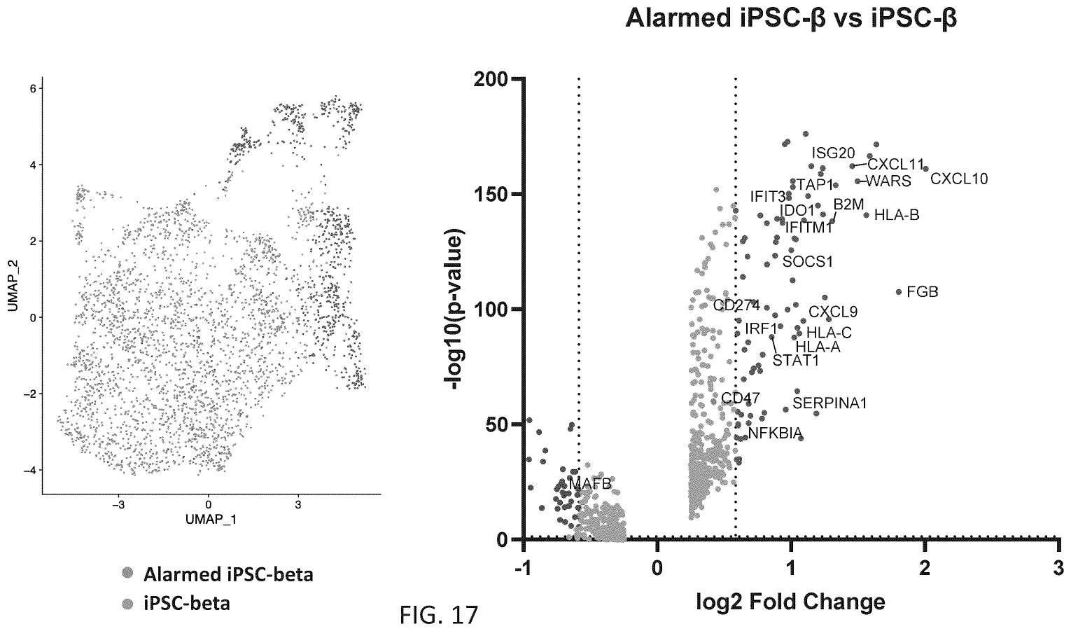

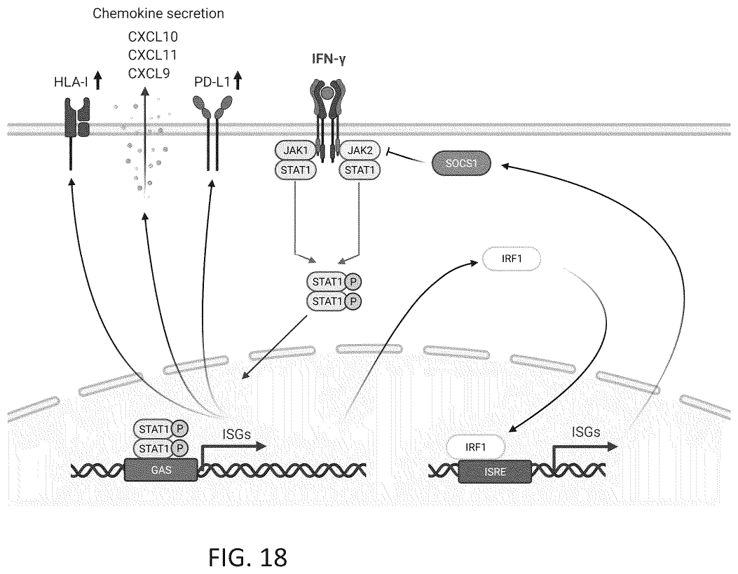

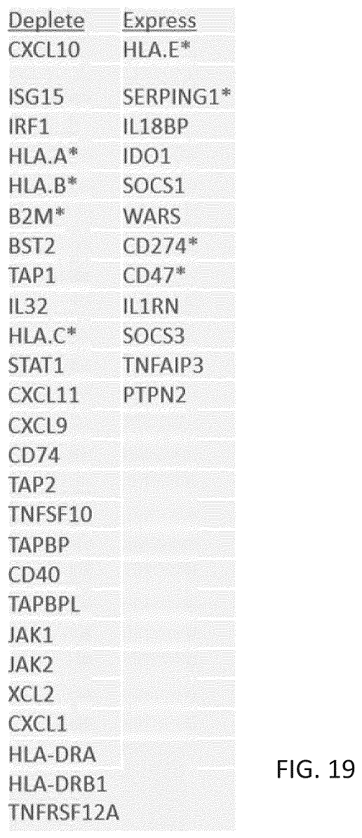

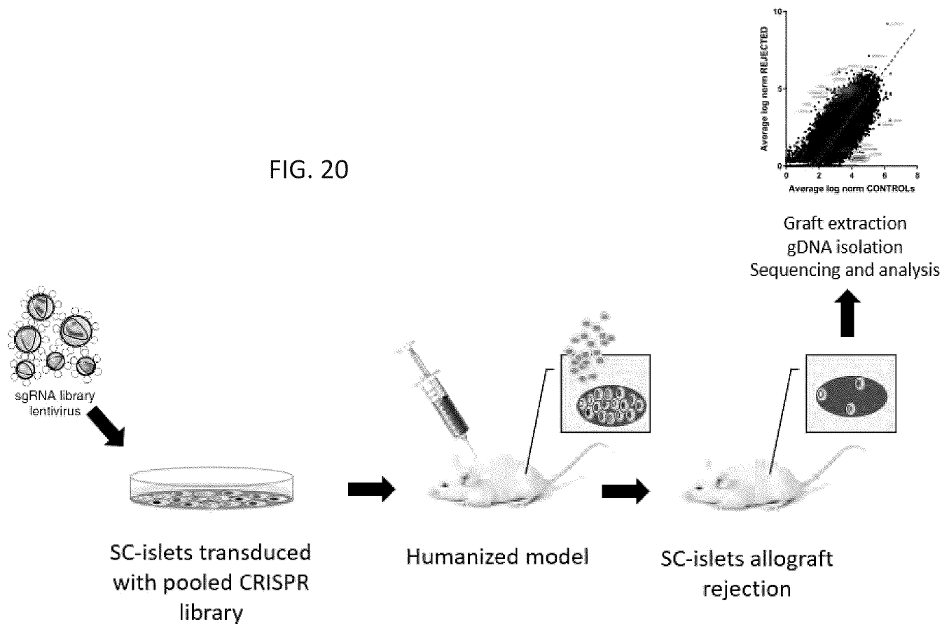

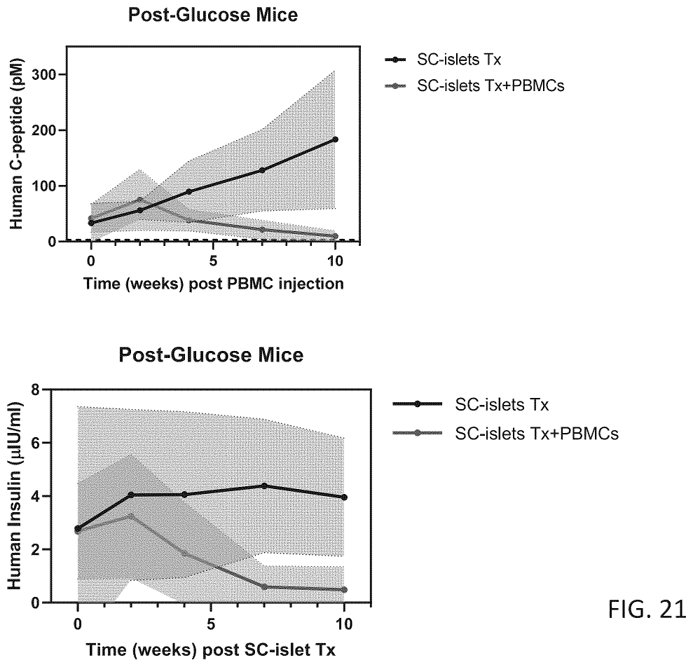

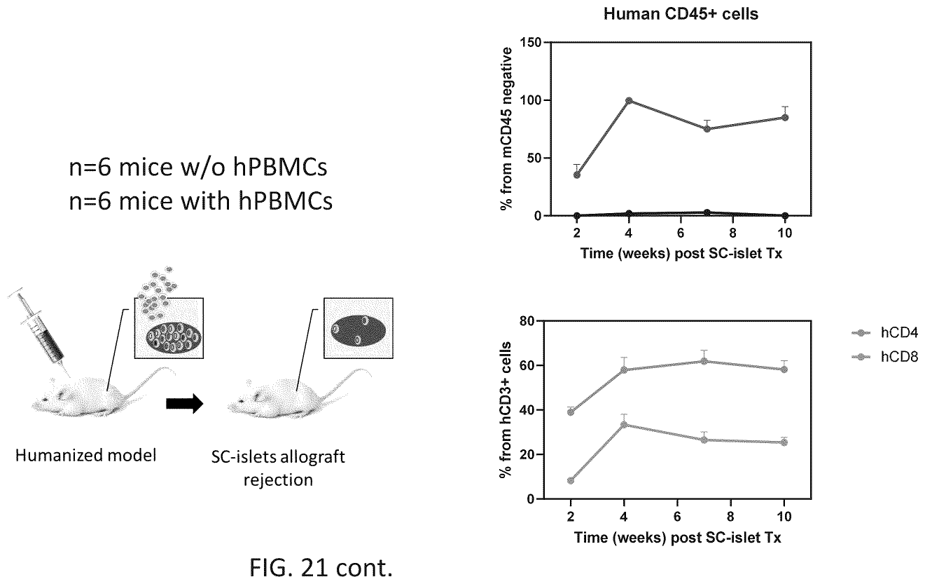

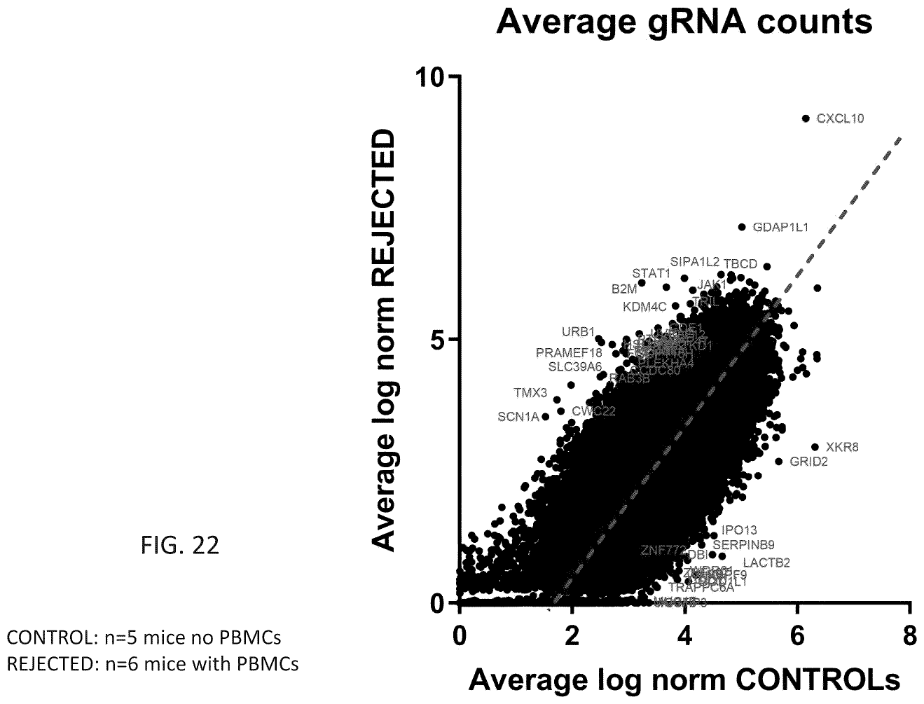

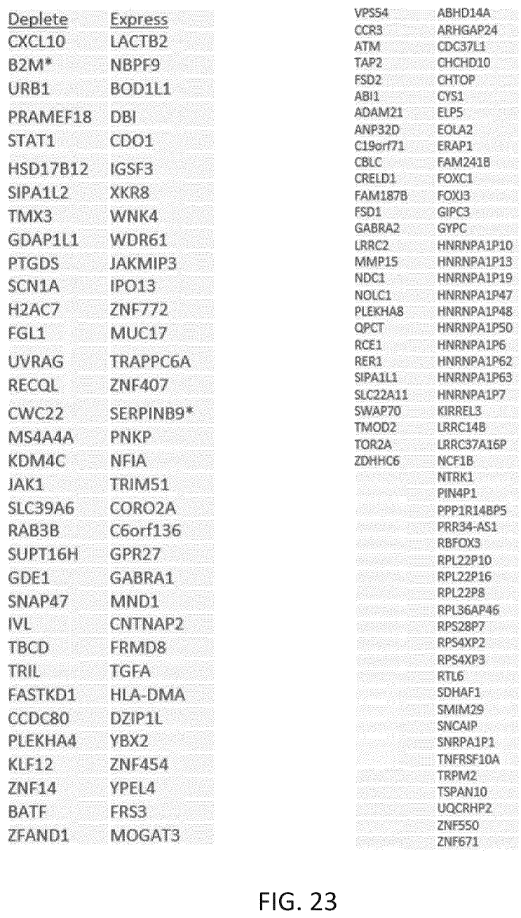

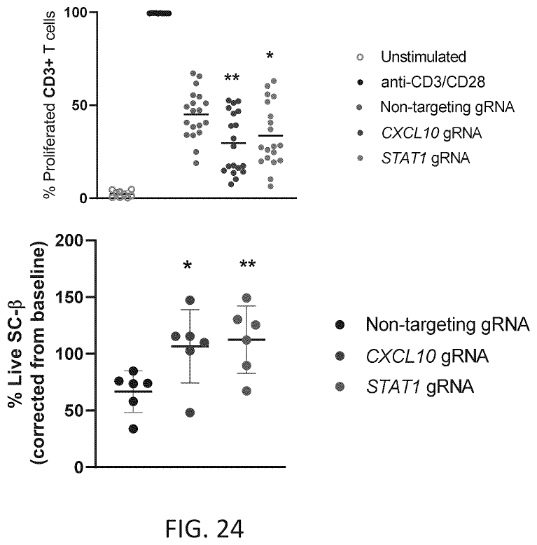

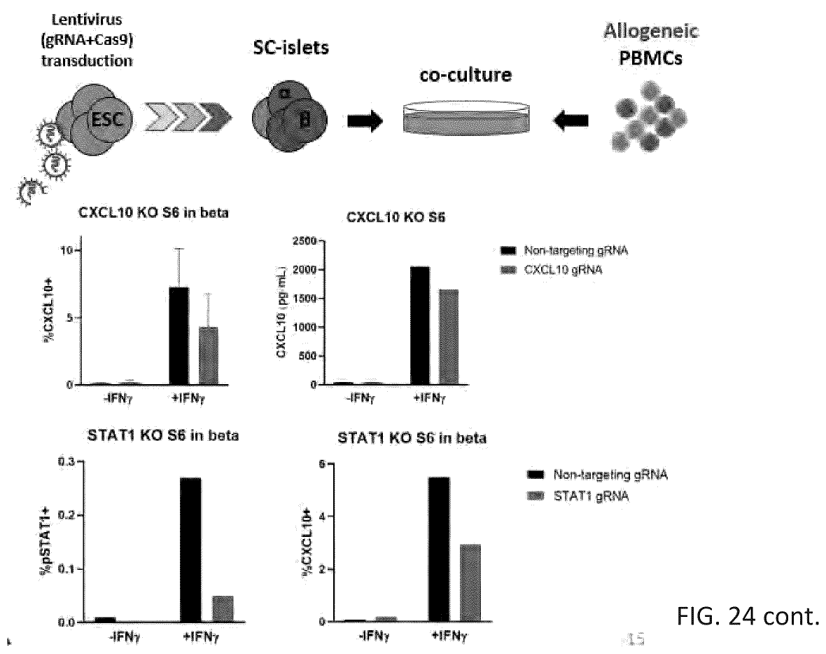

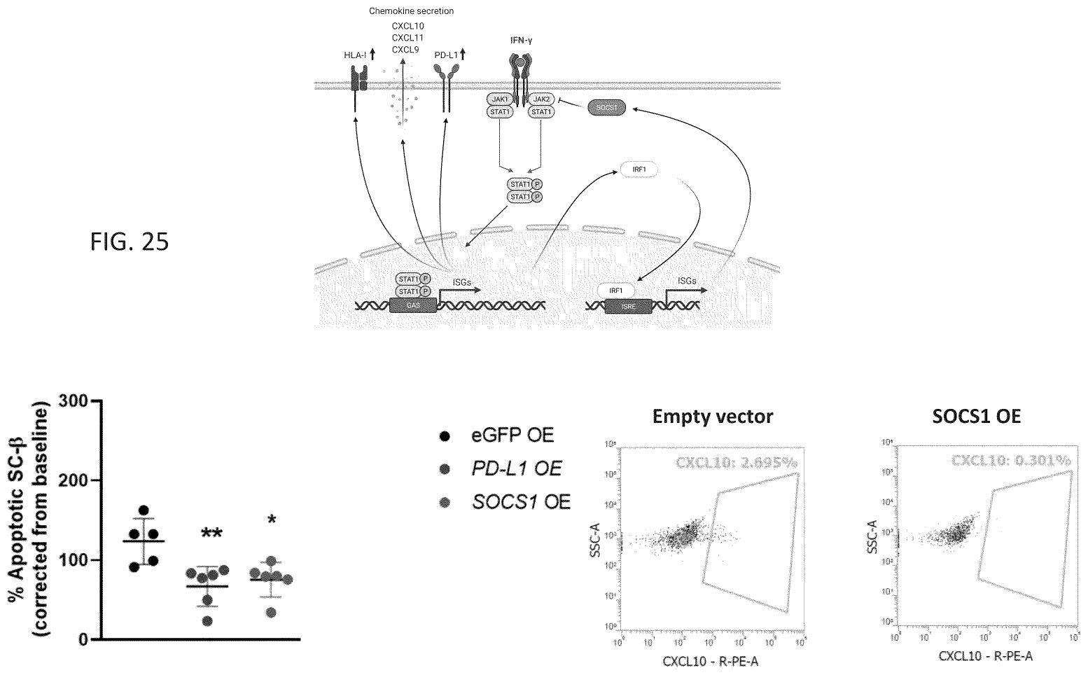

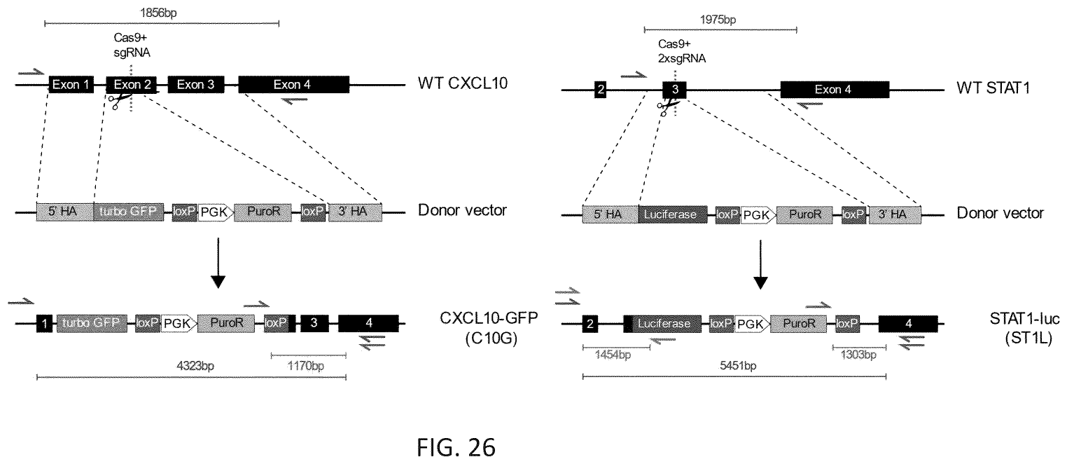

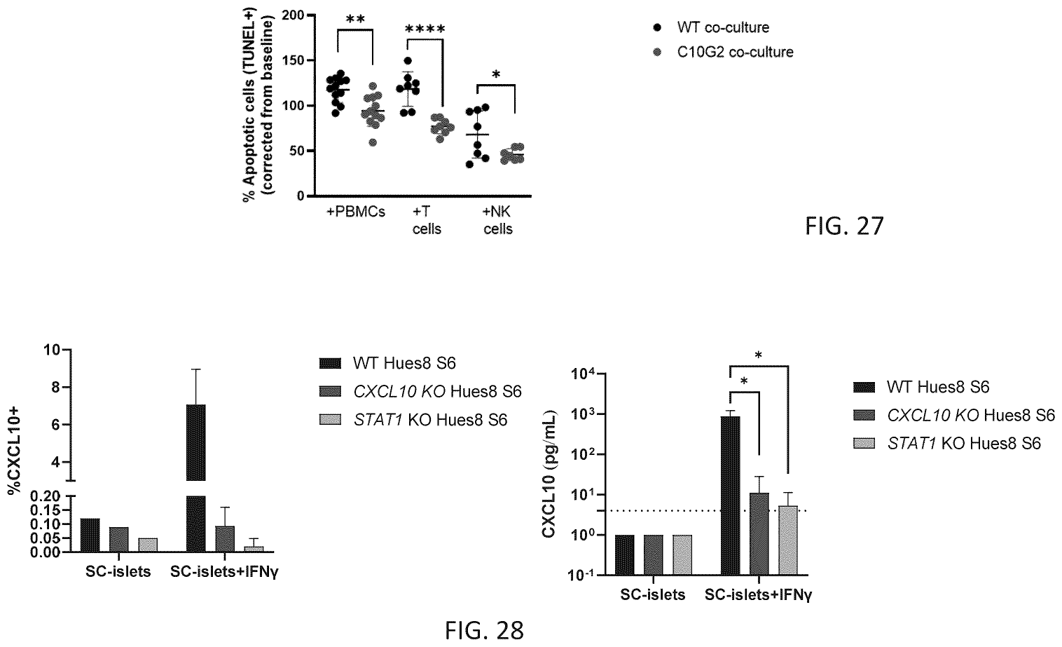

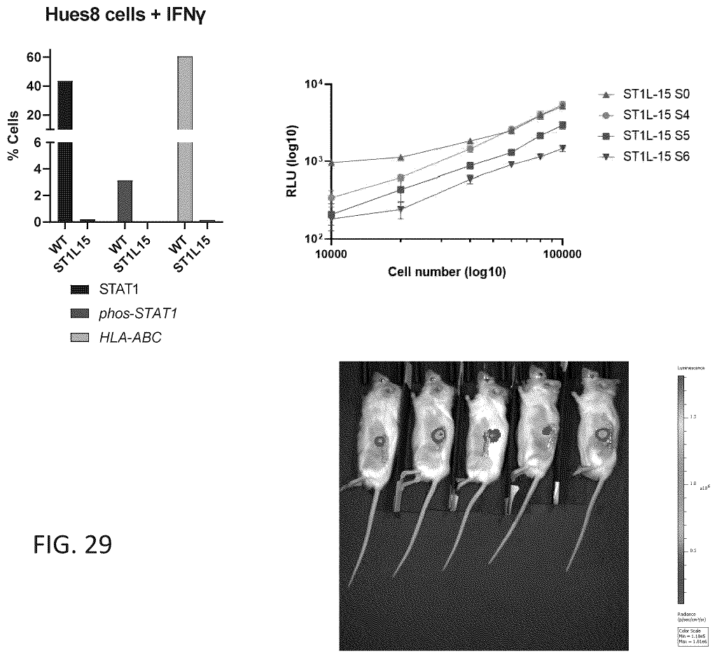

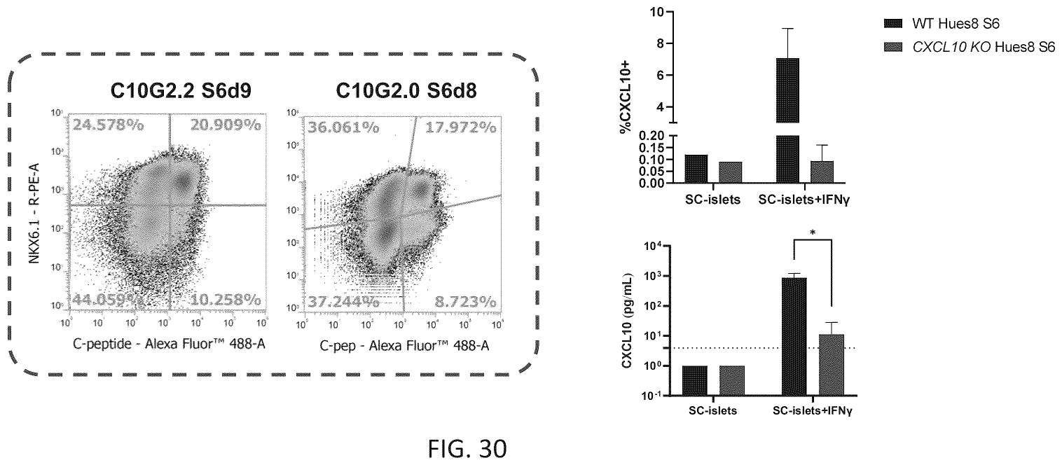

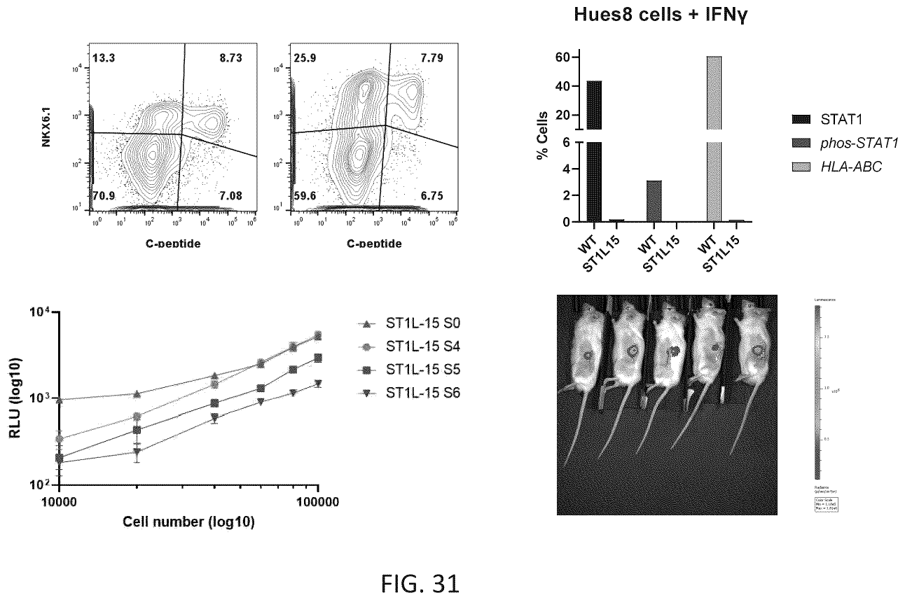

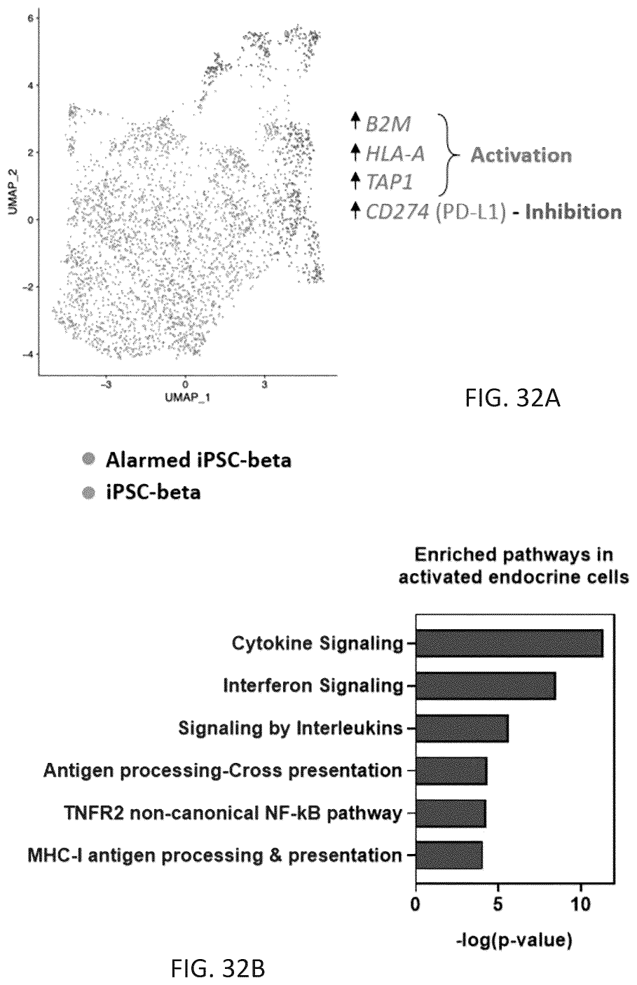

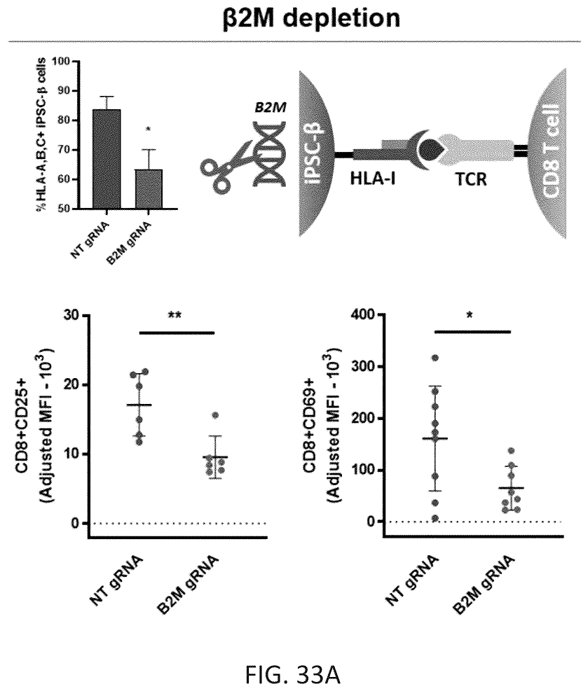

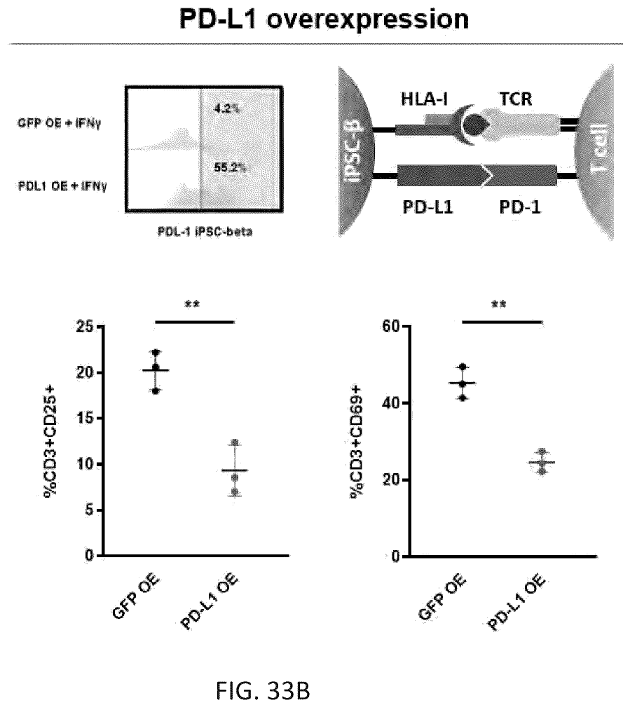

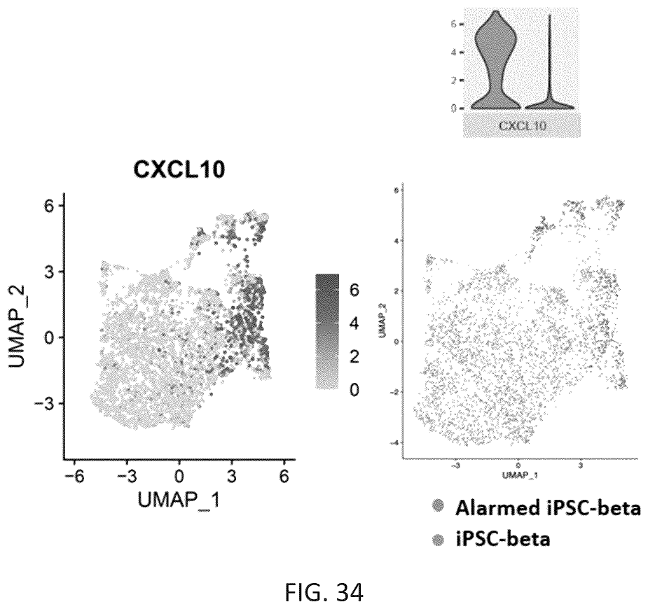

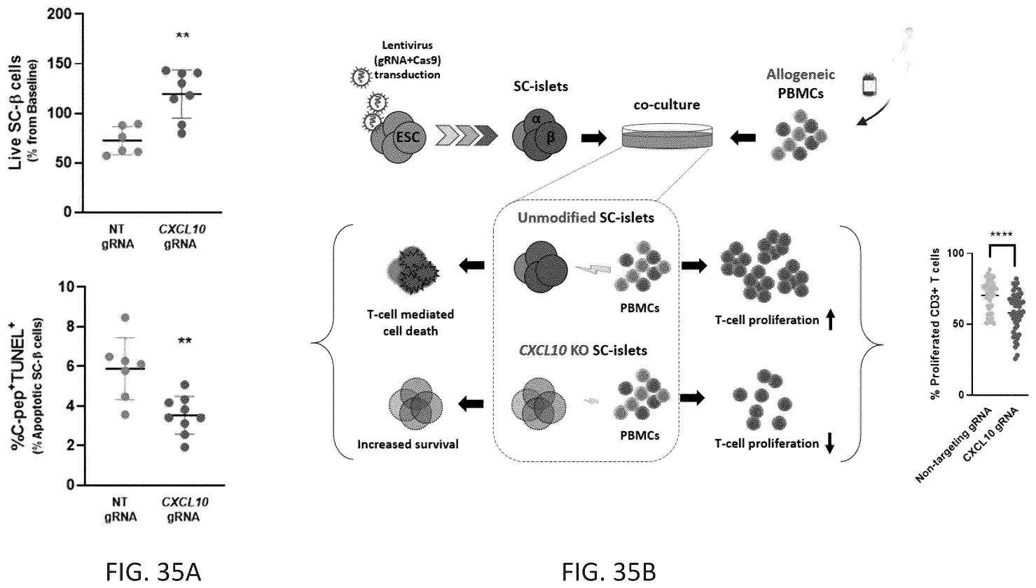

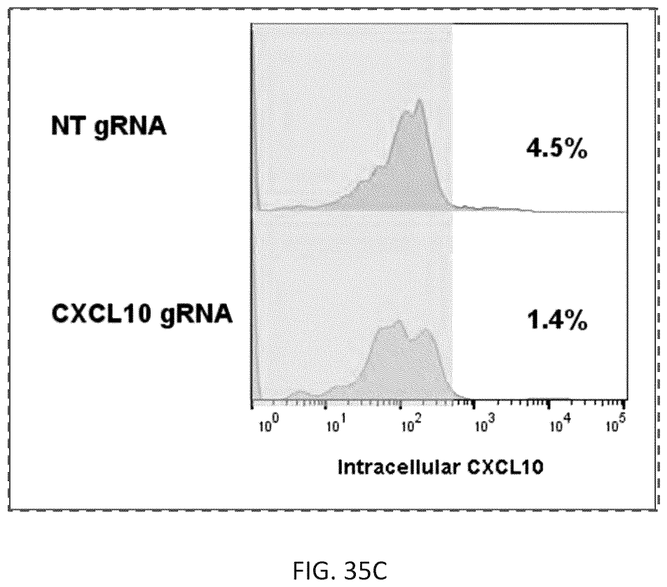

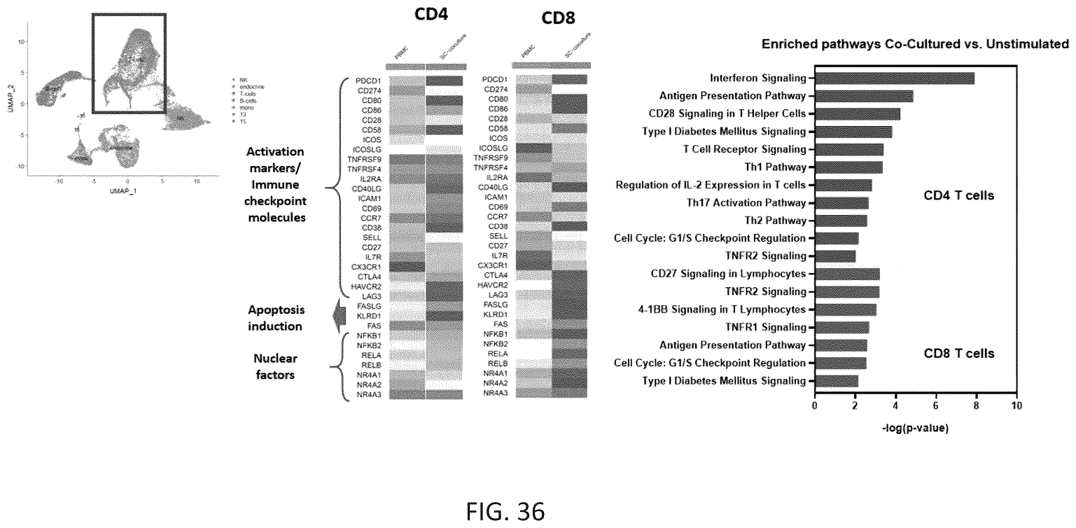

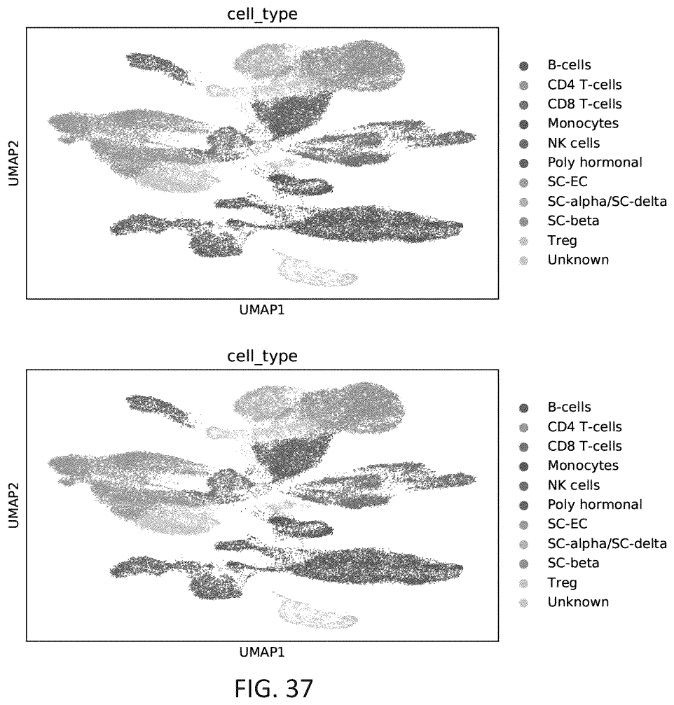

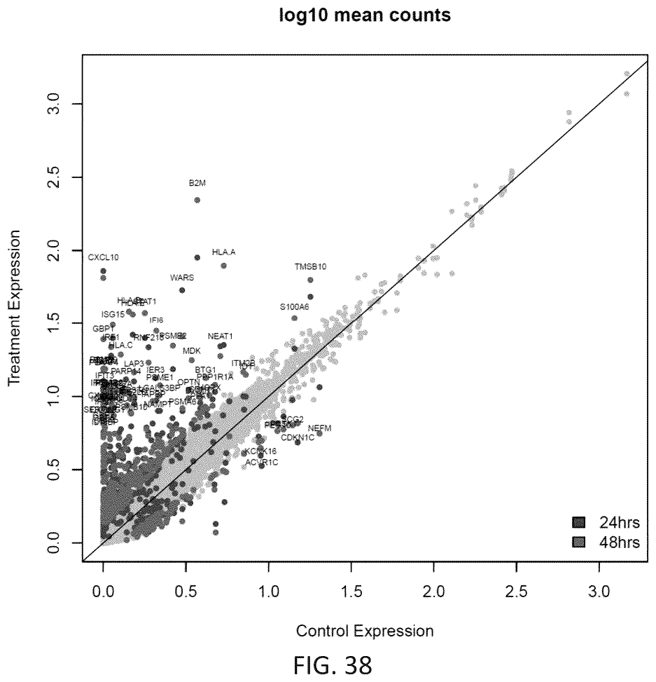

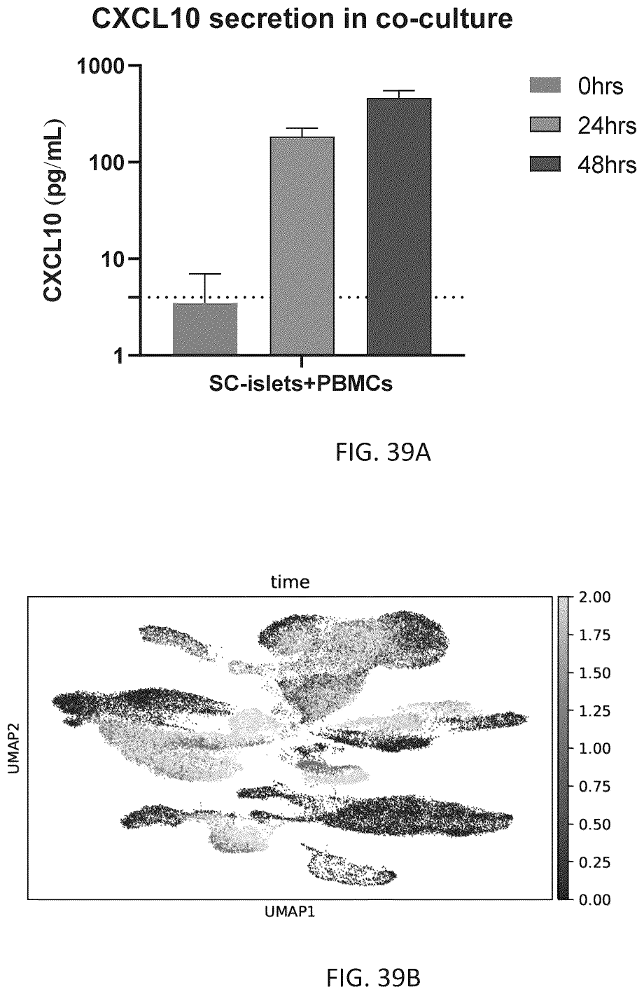

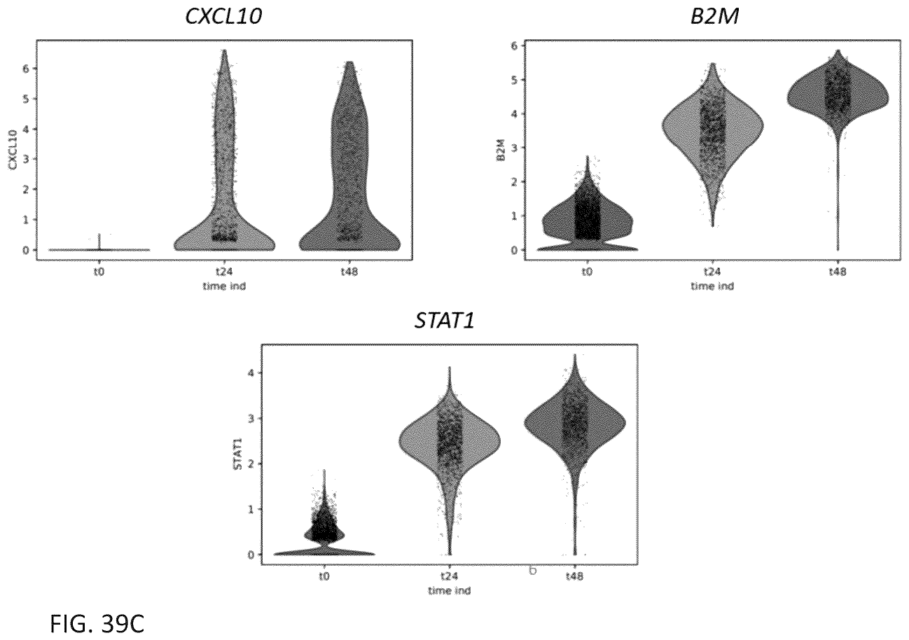

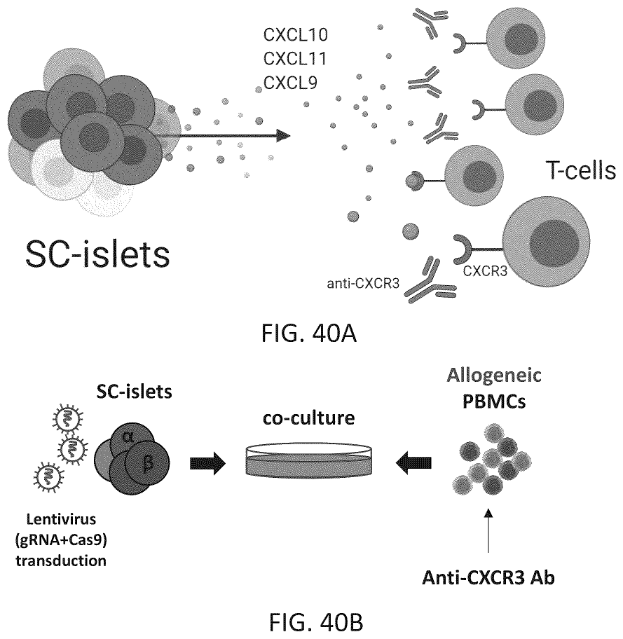

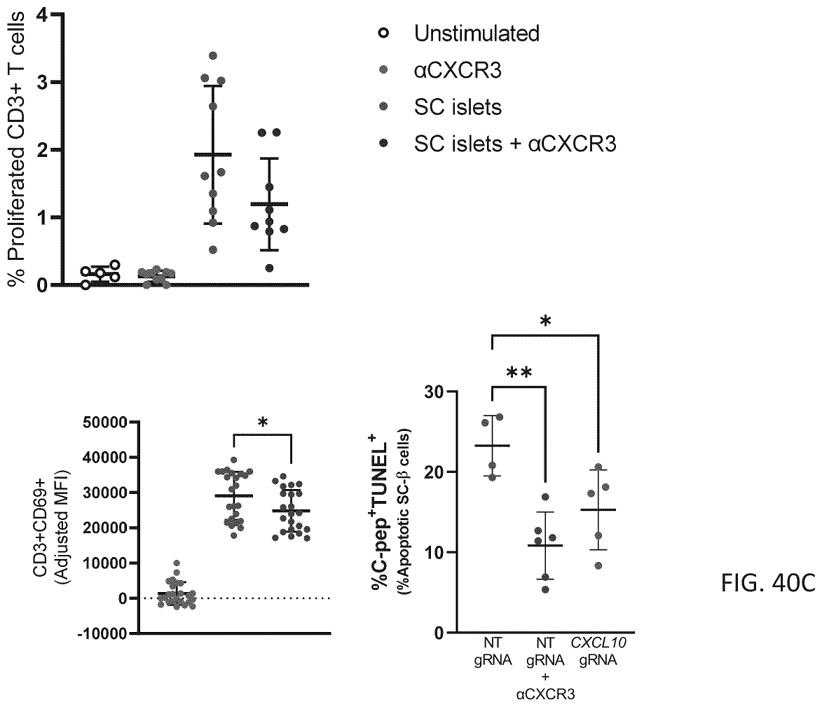

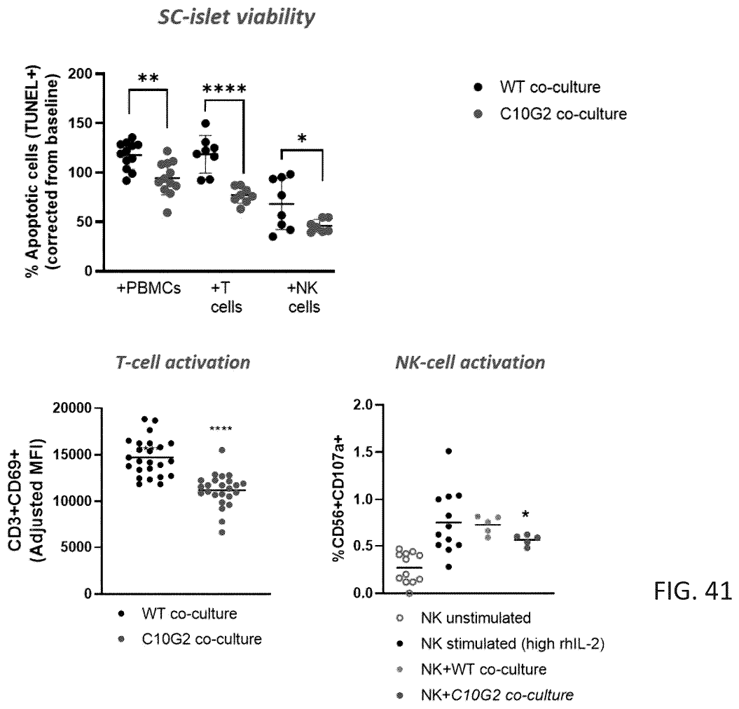

Since graft destruction was not complete and residual endocrine cells remain in the humanized mice grafts, graft extraction and scRNA-seq analysis were performed. The extracted grafts were dispersed into single cells and enriched for human cells using mouse cell depletion magnetic beads. These samples in addition to pre-transplanted SC-islets and pre-injected PBMCs (cryopreserved from the same batches/donors) were used for 10× Genomics 3′ Chromium expression library preparation and Illumina NovaSeq sequencing was performed. After filtering and removal mouse kidney cells were used for analysis. Data sets were then integrated from multiple graft and cell samples using Seurat. As seen in UMAP plots (FIG. 1H and FIG. 7C), integrated grafted endocrine cells (SC-Endo) from control and humanized mice appeared to have maintained their cell identity based on gene markers for SC-α (INS-GCG+), SC-β (INS+GCG−) and recently identified (Veres et al., 2019)SC-enterochromaffin cells (SC-EC; TPH1+). Humanized mouse grafts consisted of lower endocrine cells numbers (FIG. 7B) compared to control grafts (˜50% reduction) which is consistent with what was observed via flow cytometry staining (FIG. 1F). Next, pseudobulk differential expression analysis was performed of the different SC-Endo types, comparing engrafted cells from humanized vs. control mice. SC-α, SC-β and SC-EC exhibited similar patterns of upregulated genes in immune infiltrated (humanized) grafts, which only differs by expression levels compared to control grafts (FIG. 11 and FIG. 7D). This suggests that the response is not exclusive to a certain cell population within SC-islets, and all cells contribute in some level to its progression. Among most top upregulated, were noticeable transcripts involved in antigen processing and presentation (B2M, HLA-A,-B,-C,-F, TAP1/2, CD74), inflammatory pathway mediators (STAT1, JAK1/2, IRF1/2) and proinflammatory cytokines like IL32. These transcripts, when translated to proteins are known to induce T cell activation and contribute to the inflammatory environment. In addition, upregulated genes were identified that are inhibitory to the immune system, i.e., HLA-E, SOCS], CD274 (PD-L1), WARS, and CD47. Expression of these genes is evidence of an induction of interferon type I (IFNα/β) and II (IFNγ) pathways, through JAK/STAT signaling. Other genes play diverse roles in conjunction (e.g., PSMB9, PLAAT4, ISG20) to these pathways. (FIG. 11 and FIG. 7D). Pathway analyses (Panther/Reactome) and Gene Ontology (GO), confirms the SC-islet response as IFN-driven, one that ‘alarms’ the immune system through antigen presentation and cytokine secretion while leading to apoptosis of target cells (FIG. 7D). As IFN induced genes are upregulated, a few were chosen to focus on during the following experiments (FIGS. 4-6) and their expression was examined in SC-Endo in this analysis (FIG. 1J), including the IFNγ receptor gene IFNGR1, despite the lack of apparent change in humanized mouse grafts.

In correlation to the alarm state of SC-islet cells, human CD4 and CD8 T-cells, identified in humanized mice's grafts scRNA-seq data, exhibited expression profiles of activation (e.g., CD69, CD40LG), cytokine and chemokine signaling (e.g., IFNG) and killing (CD8 CTLs by GZMB, aka granzyme B) when compared to native PBMCs (pre-injected).

SC-Islets are Responsible for Early-Stage Immune Cell Activation Through ‘Alarm’ Genes as Learned from Single Cell Transcriptional Analysis In Vitro

As much as the use of the humanized mice model used in this study proved powerful to recreate an allogeneic immune attack that led to reduction and SC-islet mass a loss of function, it has several major limitations (Shultz et al., 2019) to this study: 1) from total PBMC injected, NK cells, are absent in long term experiments and the model is limited to T-cell responses; and 2) due to the HLA matching variability of human donors to Hues8 SC-islets, the dynamics of graft rejection differs between donors' PBMC that were used. Thus, it was found to be hard to pinpoint the exact timing of when genes that define progression of graft rejection may appear.

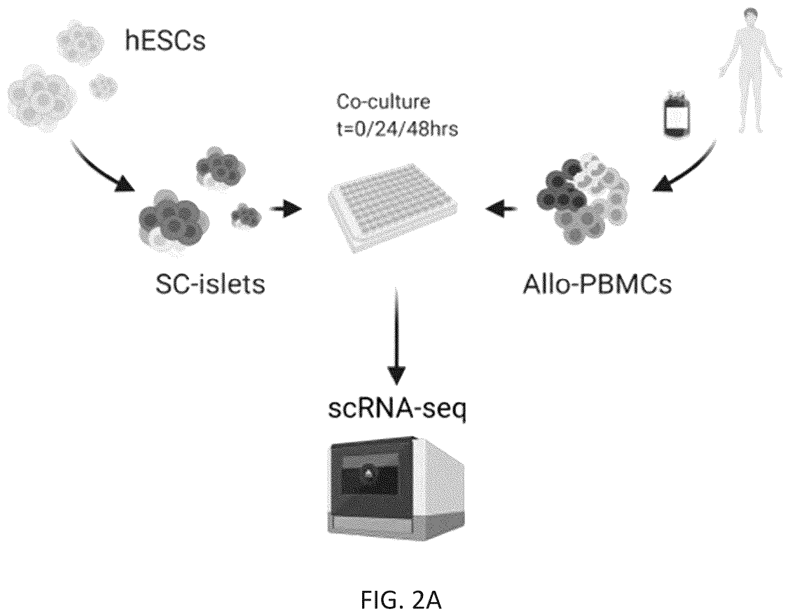

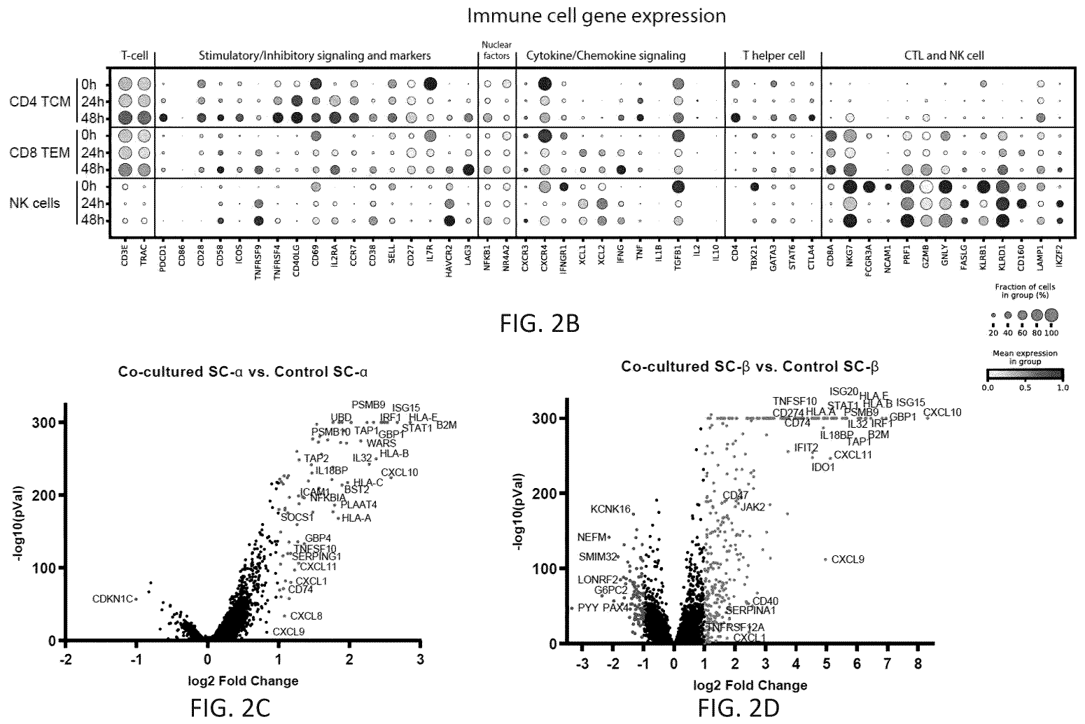

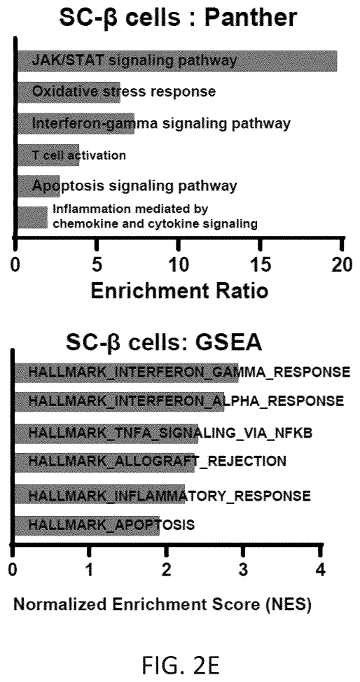

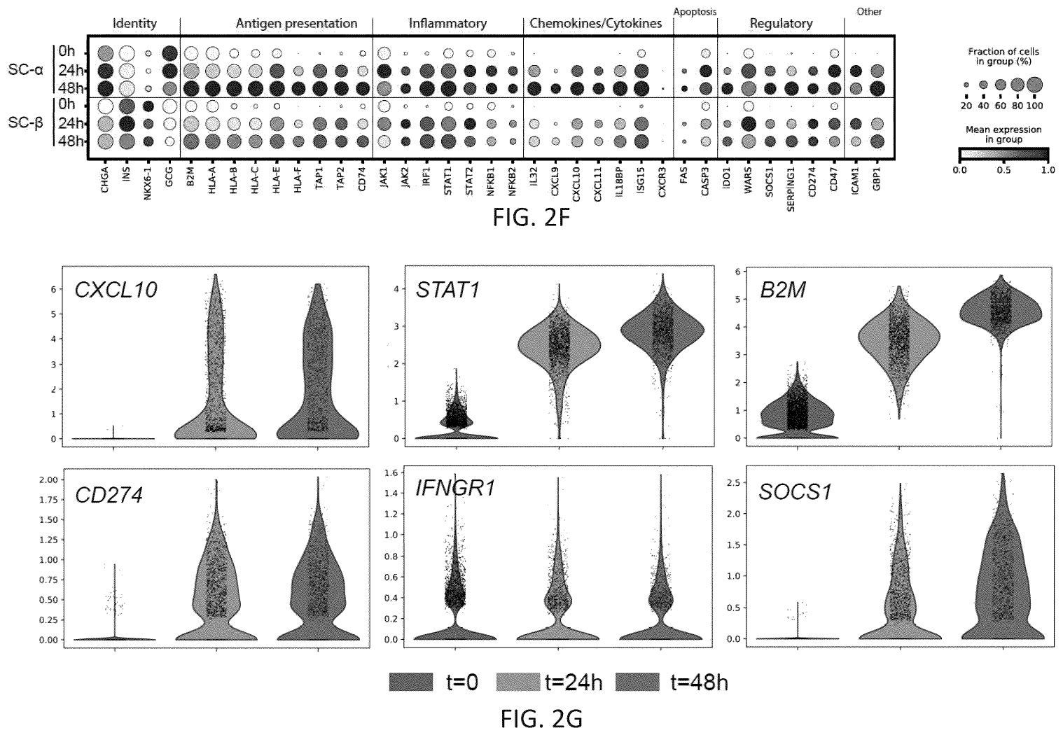

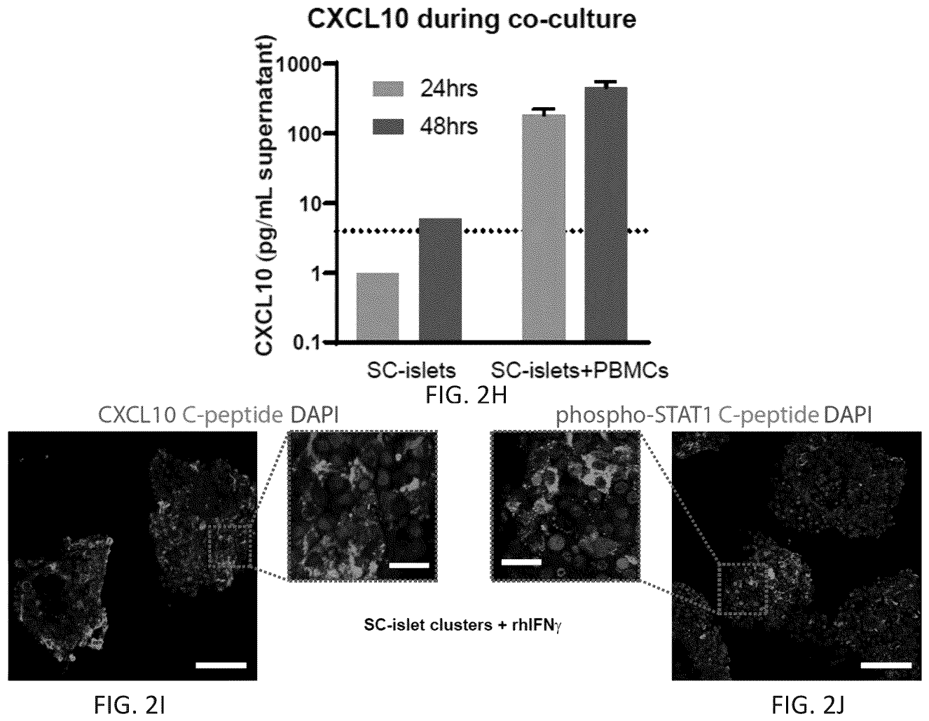

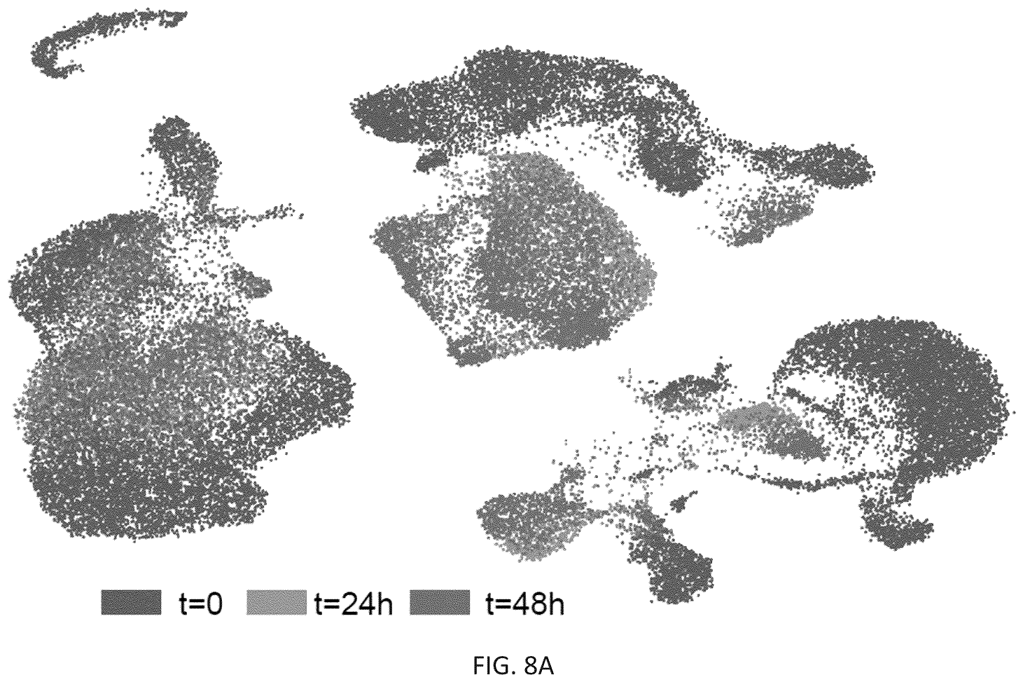

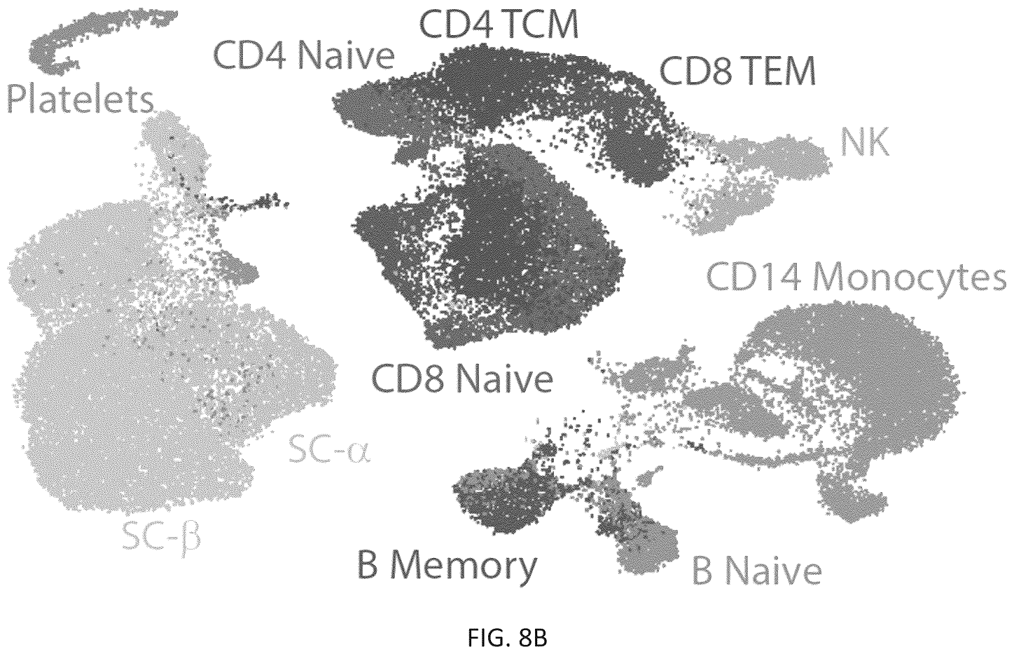

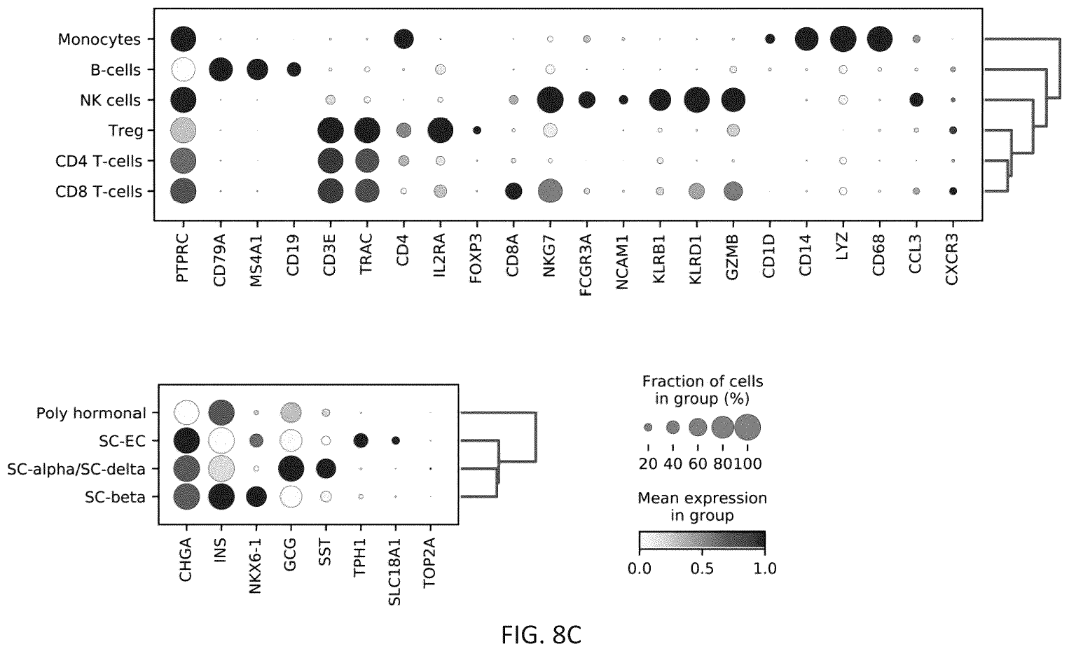

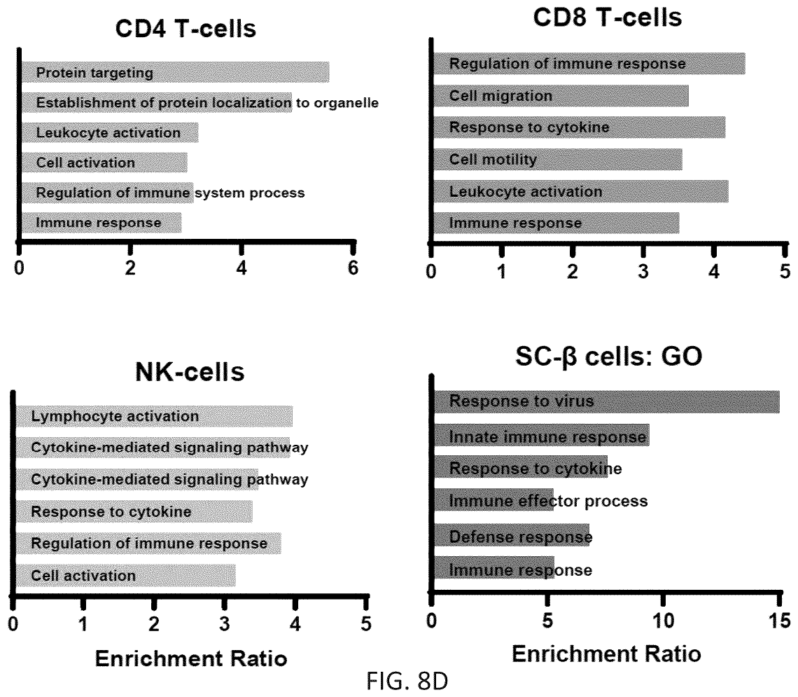

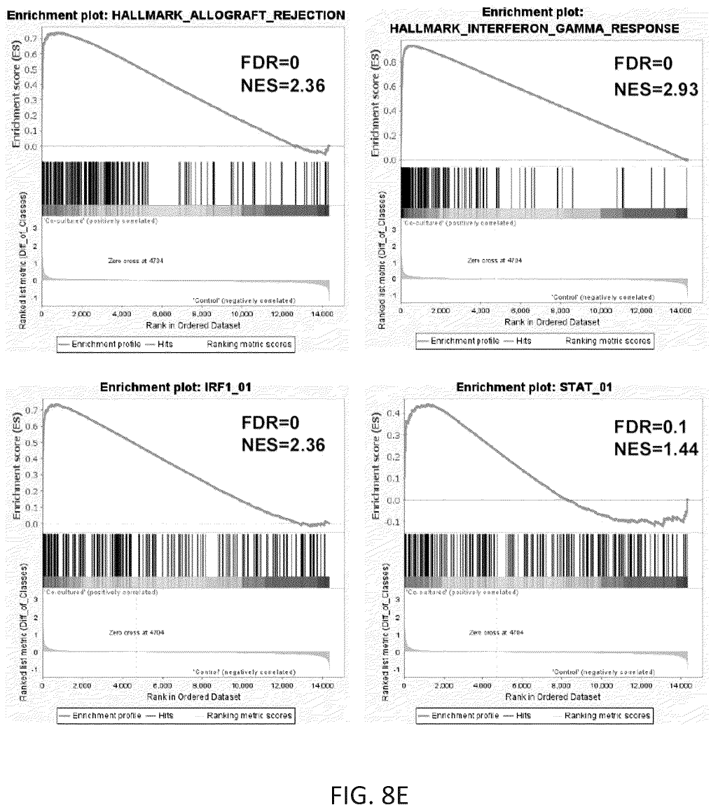

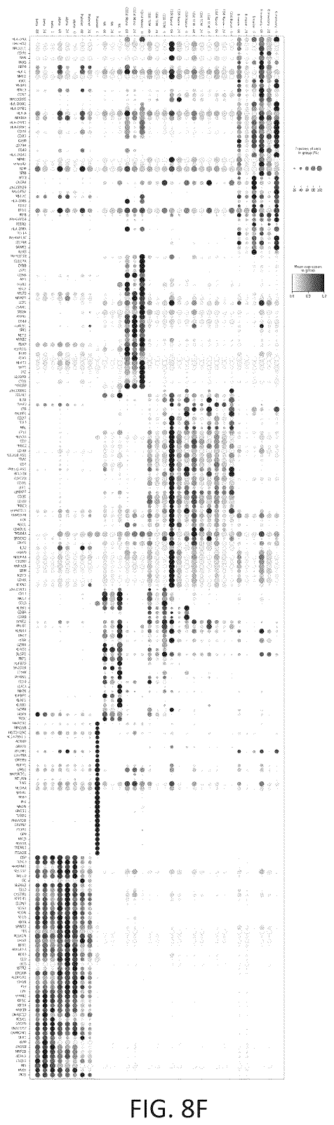

Therefore, an in vitro experiment was designed and executed, in which SC-islet clusters were SC-β enriched (using CD49A magnetic sorting to deplete non endocrine and undifferentiated cells) as previously described (Veres et al., 2019), dissociated and allowed to reaggregate in V-shaped 96-wells to acquire a uniform count between wells. SC-islets were then co-cultured with human allogeneic PBMCs for 24 and 48 hours. As controls (t=0), SC-islets remained in culture without PBMC addition. These samples, in addition to the PBMCs without co-culture, (t=0) were used for 10× Genomics 3′ Chromium expression library preparation and sequencing (FIG. 2A). PBMCs originated from the same donors used in vivo (FIG. 1). Prior to co-culture all SC-islets were pre-treated with thapsigargin to induce ER-stress in endocrine cells. As previously reported, ER-stressed SC-islets can induce and accelerate T-cell activation (Leite et al., 2020). Differential expression analysis of integrated data from all samples was performed focusing on cell populations of interest as defined by gene markers (FIG. 2 and FIG. 8).

When compared to non co-cultured samples, CD4 and CD8 T-cells as well as NK cells at 24 and 48 hr co-cultures displayed gene expression profiles of immune activation (FIG. 2B and FIG. 8D). Genes that transcribed to T-cell co-stimulation molecules (such as CD28, CD58 (LFA-3), CD40LG, TNFRSF9 (4-1BB), TNFRSF4 (OX40)) and other activation markers (CD69, IL2RA (CD25), CD38) are upregulated in T-cells as well as inhibitory and exhaustion markers (HAVCR2 (TIM-3), LAG3, PDCD1 (PD-1) (FIG. 2B, left)). Co-inflammatory cytokines (IFNG and TNF) and chemokines (XCL1/2) are expressed over time in NK and T cells, while anti-inflammatory cytokines (IL10 and TGFB1) are either undetected or down regulated. T-cells and NK sensitization to pro-inflammatory chemokines is increased based on elevated levels of CXCR3, a chemokine receptor that binds CXCL9/10/11 (FIG. 2B, center). Other prominent transcripts are those that play part in CTL and NK killing functions (FIG. 2B, right: PRF1 (Perforin), GZMB (Granzyme B), FASLG), further emphasizing their role in allogeneic attack in the co-culture system.

Due to the CD49A enrichment performed prior to the co-culture experiment, high numbers of SC-α, and SC-β, but very low number of SC-EC cells, were detected. Therefore, the differential expression of co-cultured SC-α and SC-β cells was focused on, compared to controls without PBMC addition. Similar to what was observed in the in vivo analysis (FIG. 1), in the co-culture experiment, upregulated profiles did not differ between co-cultured SC-α and SC-β cells (FIGS. 2C-2D) and consisted of clear IFN responses through the JAK/STAT pathway with implications to T-cell activation, apoptosis signaling and allo-rejection (FIGS. 2E-2F, FIGS. 8D-8E, pathway analysis and GSEA). Unlike the in vivo experiment, in co-culture of a short time frame of 24-48 hrs, chemokine signaling was most eminent with CXCL10 as the top upregulated gene in SC-islets after co-culture (FIGS. 2C-2D and FIG. 2F, center) along with the same chemokine family members CXCL9 and CXCL11. Chemokine signaling plays an important part in attracting immune cell recruitment to an inflamed tissue and may be attributed to an early inflammatory response that was missed in the graft harvesting timing of the humanized mice model (FIG. 1). CXCL10 (IP-10) is also an IFN induced protein, which according to the data, peaks at 24 hr of co-culture (FIG. 2G) while other IFN-induced genes maintain or increase their levels, coming to the 48 hr time frame (FIG. 2G) and even a 10-week frame in vivo (FIGS. 1I-IJ) under immune attack. After 10 weeks in humanized mice grafts, CXCL10 is still upregulated but very few SC-islets still express it (FIG. 7D and FIG. 7F). While it accumulates in the co-culture media (FIG. 2H) and detected in endocrine cells in SC-islet clusters under inflammatory signals (FIG. 2I), it was concluded that CXCL10 plays a pivotal role in T-cell and NK cell stimulation that needs to be fully assessed.

As the entire JAK/STAT pathway is highly upregulated in SC-islets during co-culture with PBMCs, genes that mediate it starting from the IFNγ receptor gene IFNGR1, intracellular regulator STAT1, negative regulator SOCS1 and additional downstream effectors B2M (activator via antigen presentation) and CD274 (PD-L1 co-inhibitor surface molecule) were focused on (FIG. 2G). STAT1 is known to be a master regulator of the JAK/STAT pathway (Gurzov et al., 2016) and is enriched in the GSEA transcription factor motif analysis (FIG. 8E). As further evidence for the pathways importance in SC-islet immunogenicity, it was shown that upon IFN stimuli, STAT1 is phosphorylated and translocated into the nuclei of SC-islets (FIG. 2J), where it induces transcription of IFN response elements, including CXCL10 (Moore et al., 2011).

Whole Genome CRISPR Screens Confirm Components of the IFN Response as Candidates for Perturbations that Enhance SC-Islet Survival

The transcriptional profile of SC-islets under immune induced inflammatory environment as presented here, may provide active genes that can be targeted to achieve reduction in immune surveillance or recognition. But an expression of a given gene could either support a 1) pro-stimulatory, 2) anti-stimulatory immunological drive toward destruction of islet cells, or 3) balanced effect or no effect. A validating system was required to filter and assess what was learned from single cell RNA-seq data (FIGS. 1-2). Thus, a whole genome Brunello CRISPR lentivirus library (Doench et al., 2016) was applied to a screen for gene edits that could affect SC-islets survival in an allogeneic attacked/rejected graft. The Brunello library consists of a pool of 76,441 human targeting guide RNAs (gRNA) and 1000 control gRNAs (non-targeting or intergenic), in a lentiviral vector (lentiCRISPRv2) that also expresses Cas9. The pooled library targets 19,114 human genes, most of them by four gRNA per gene.

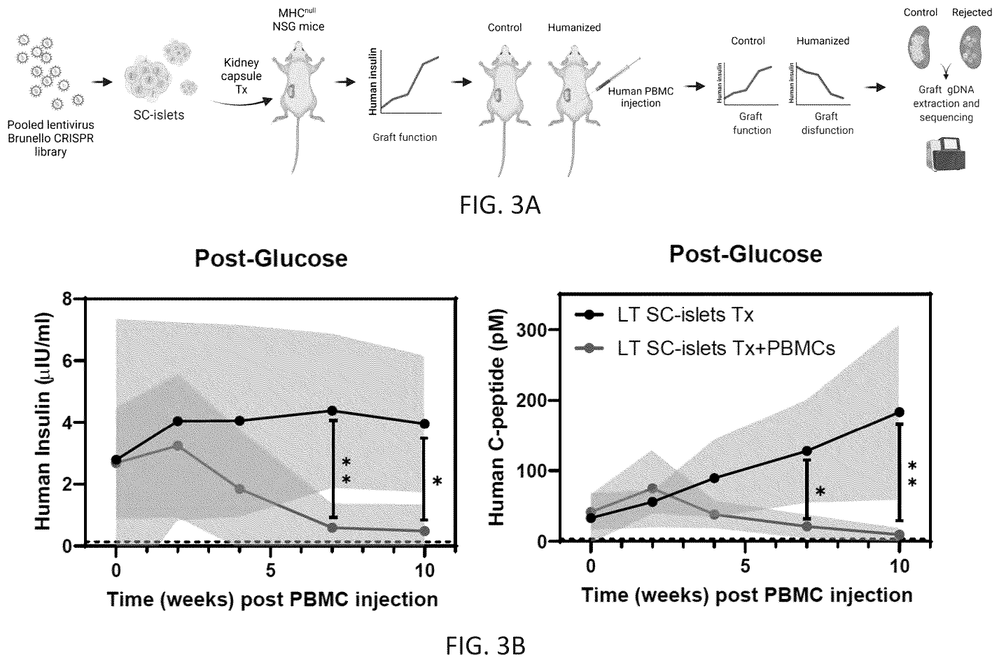

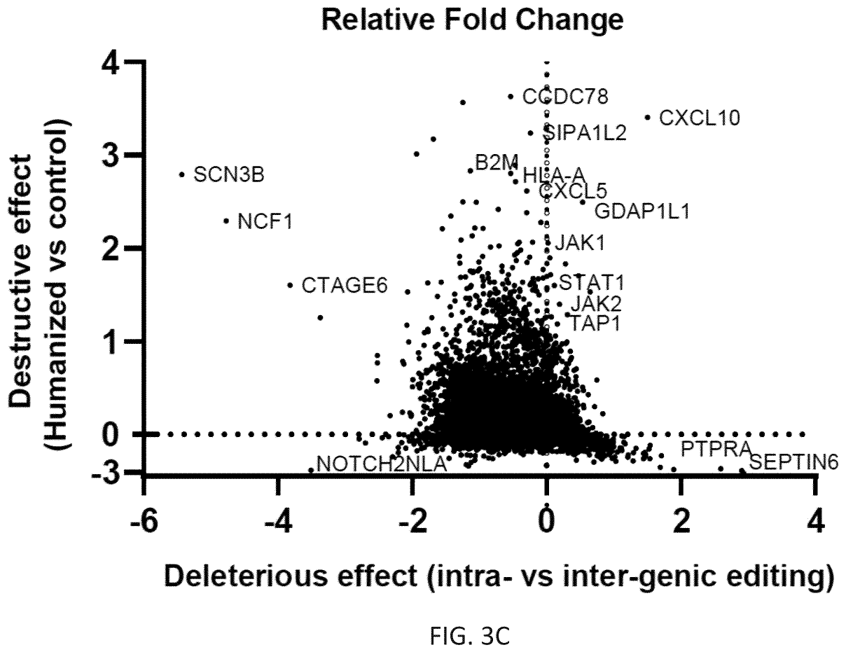

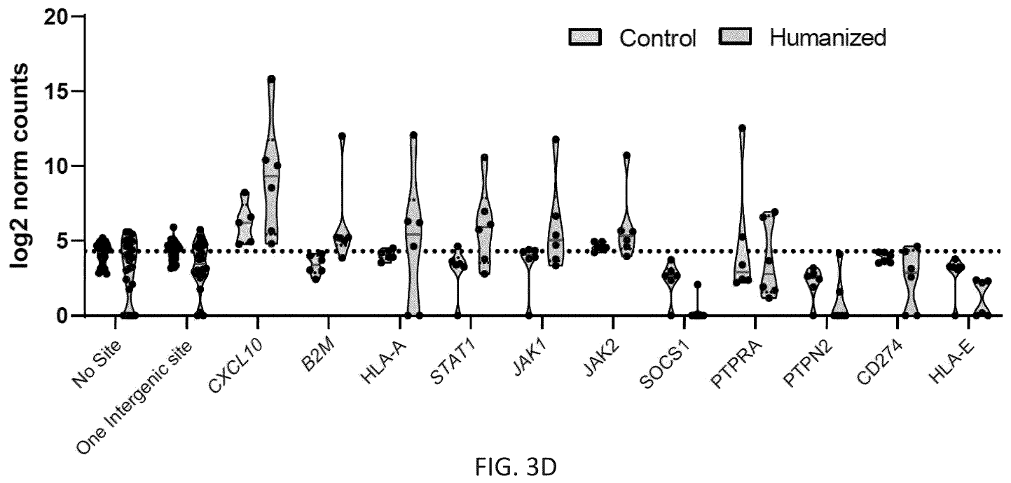

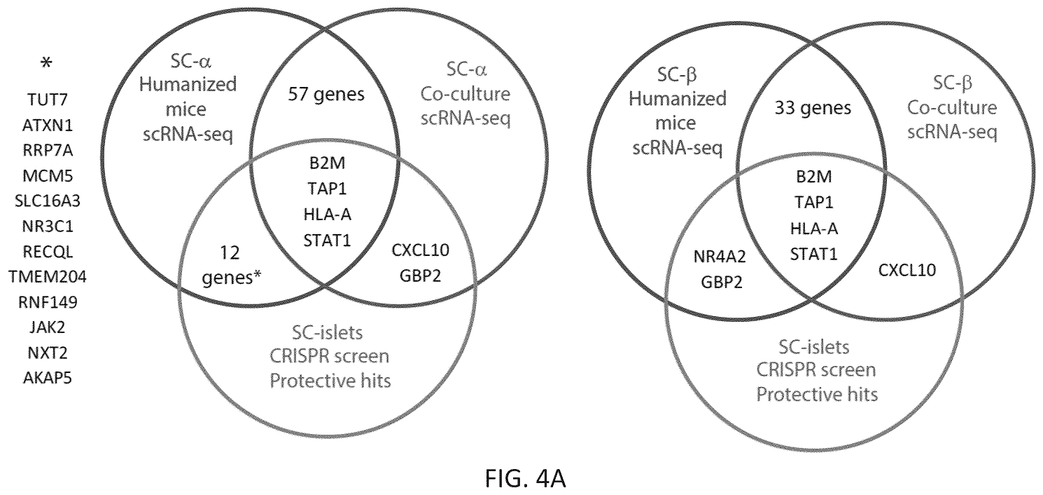

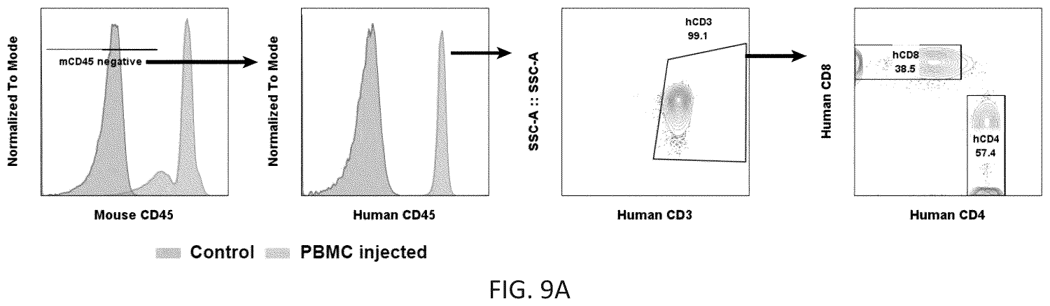

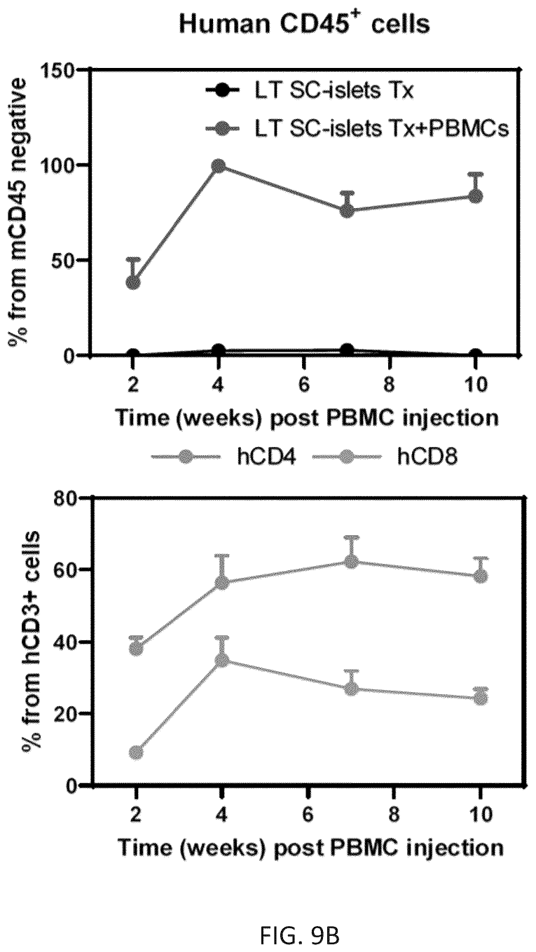

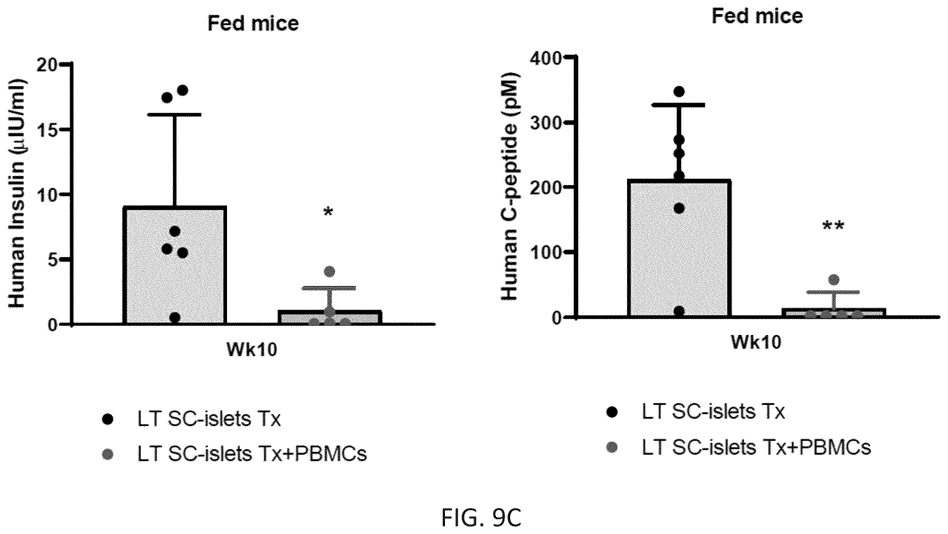

To avoid multiple different gRNA in cells and a nonspecific effect on the screen results (Doench, 2018), a low infection lentivirus titer (multiplicity of infection that is <1) was used to transduce dissociated SC-islets with the Brunello library. Library transduced cells (LT SC-islets) were then allowed at least 10 days for optimal CRISPR editing and reaggregation of cell clusters, before transplantation to the NSG-MHCnull humanized mouse model (FIG. 3A). Graft function and subsequent failure (due to human PBMC injection) (FIG. 1) was determined by human insulin and C-peptide detection from (fasted) mouse blood 30 min after glucose administration (FIG. 1B), and from non-fasted mice at the 10-week end point (FIG. 9C). Humanized mice kept their T-cell circulating level throughout the experiment (FIGS. 9A-9B). When graft failure was confirmed 10 weeks after PBMC injection, grafts were recovered, and genomic DNA (gDNA) was extracted and amplified by PCR for Illumina sequencing.

For analysis, the perturbations' effect was assessed based on gRNA mean counts from immune attacked LT SC-islet grafts relative to control grafts, while also considering the distance from control intergenic gRNA counts. A ranking method was applied based on a Gene to Environment interaction model (Caspi et al., 2003). As seen in FIG. 3C, axis are z-scores of relative fold changes between average counts of 4 gRNA targeting a given gene, in respect to the condition (y-axis: Humanized vs control mice) and/or in respect to intergenic gRNA mean counts (y-axis and x-axis). Thus, in the top left quadrant, each dot is a gene that when expressed, based on its distance from 0, will benefit graft survival in normal conditions, but upon immune attack will be harmful, e.g., once perturbed, NCF1 is a strong hit for graft survival against immune destruction but is essential under normal conditions and perturbing it might have negative effect upon transplantation. Although in the same quadrant, B2M and HLA-A perturbations are beneficial to cell protection under immune challenge, they will have little effect on graft viability in general. This aligns with previous reports on the protective effect of HLA-I knockouts that prevent allo-recognition by T-cells (Castro-Gutierrez et al., 2021; Han et al., 2019; Parent et al., 2021; Wang et al., 2015). In co-ordinance, the hits in the top right quadrant are genes that are deleterious both upon normal grafting and under PBMC injection and infiltration. Strikingly, one of the top hits in this quadrant and on the positive y-axis in general, is CXCL10 perturbation. This means that CXCL10 expression in SC-islets can be harmful when confronted with allogeneic immune responses, modeled here using humanized mice. Even without PBMCs, cells that might express it will have less survival capability. Therefore, depletion of CXCL10 is strongly suggested to be beneficial for SC-islet transplantations. In the context of immune protective effect, other than B2M, HLA-A, and CXCL10, other canonical IFN pathway related genes: STAT1, JAK1 and JAK2 were found. When looking at enrichment per selected gRNA of each of these genes, across mouse replicates (n=6 per group), counts of these perturbations is higher in grafts of humanized mouse compared to control mice and compare to control gRNA counts (FIG. 3D). In the case of CXCL10, the change between CXCL10 gRNA compared to control gRNAs (dashed line) in control mice, is noticeable.

Bottom quadrants in the gene to environment interaction plot (FIG. 3C) have gene hits that are beneficial to graft survival under immune infiltration of PBMCs. The gRNA targeting those genes are the most depleted in humanized mice grafts. Therefore, artificially these genes may be preventative to immune destruction. One such example is PTPRA, which belongs to the family of protein tyrosine phosphatases (PTPs) that are known to be negative regulators of JAK/STAT signaling (Gurzov et al., 2015; Stanley et al., 2015). Another member of PTPs, PTPN2, is a T1D risk gene (Barrett et al., 2009; Espino-Paisan et al., 2011) and was ranked lower as a beneficial gene in the screen (dot is not labeled in FIG. 3C) but showed lower counts in humanized mice compared to control when perturbed (FIG. 3D). In addition, suppressor of cytokine signaling 1 (SOCS]) which is also a negative regulator of JAK/STAT (Galic et al., 2014; Solomon et al., 2011) was upregulated in our scRNA-seq data (FIGS. 1-2) and exhibited potency as a tolerizing gene (FIG. 3D) in the CRISPR screen. Other protective/tolerizing molecules that were previously described: HLA-E (Gornalusse et al., 2017) and PD-L1 (CD274) (Castro-Gutierrez et al., 2021; Yoshihara et al., 2020), did show a protective effect (FIG. 3D) but surprisingly were less potent compared to the overall ranking.

### Dampening the IFNγ Pathway Affects SC-Islet Immunogenicity

scRNA-seq experiments in vitro and in vivo provided an abundant amount of data on the immune attack effect on- and by- SC-islet internal signaling (JAK/STAT) that was translated to external signals manifested by antigen presentation and chemokine secretion. The data was compared with the unbiased approach of whole genome screening and further confirmation of IFN signaling as the main player was obtained. All significant positive readouts from all assays, upregulated scRNA-seq genes and gRNAs enriched in humanized mice grafts were compared (FIG. 4A). Within the CRISPR screen hits, there are 4 common genes that are also upregulated in immune challenged engrafted or co-cultured SC-β and SC-α cells. These observed genes are strong evidence for the widely known importance of antigen processing (TAP]) and presentation (B2M, HLA-A) in the MHC class I initiation of immune responses. STAT1 links the external signal of IFNγ (also IFNα and β) receptors with the downstream effect that includes the MHC-I stimuli, but also secreted agents like CXCL10. CXCL10 was the most prominent gene in this study, found to be one of the strongest hits in the CRISPR survival screen (FIG. 3). Not many cells express CXCL10 in late rejection stages of grafts (FIG. 1), but cells that lack it gain increased survival capability in the same in vivo setting (FIG. 3), suggesting it has a role in early immune-graft interaction time points. Its vast expression in the short-term co-culture experiment supports this hypothesis (FIG. 2).

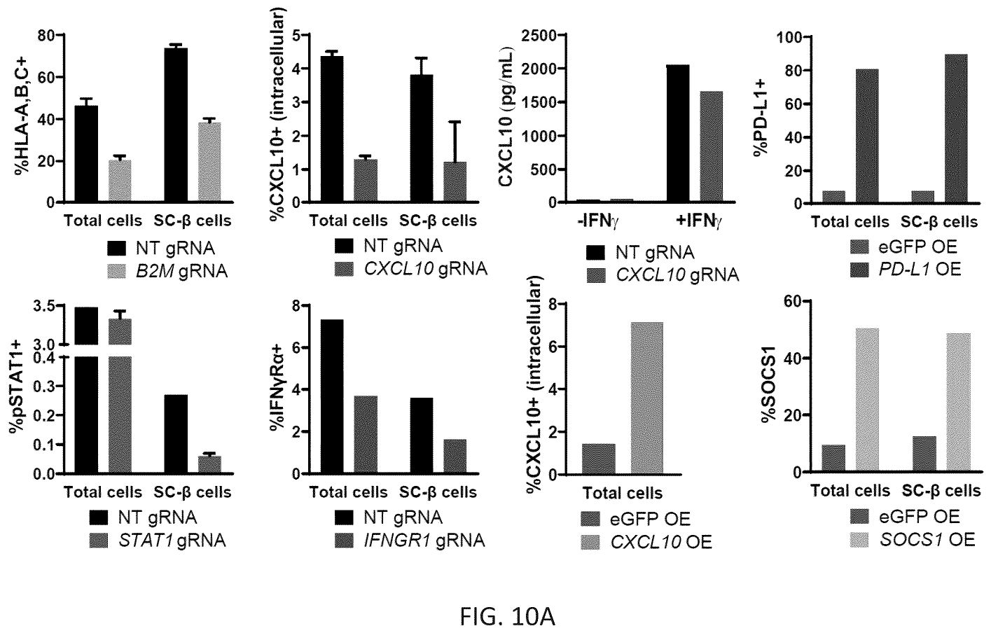

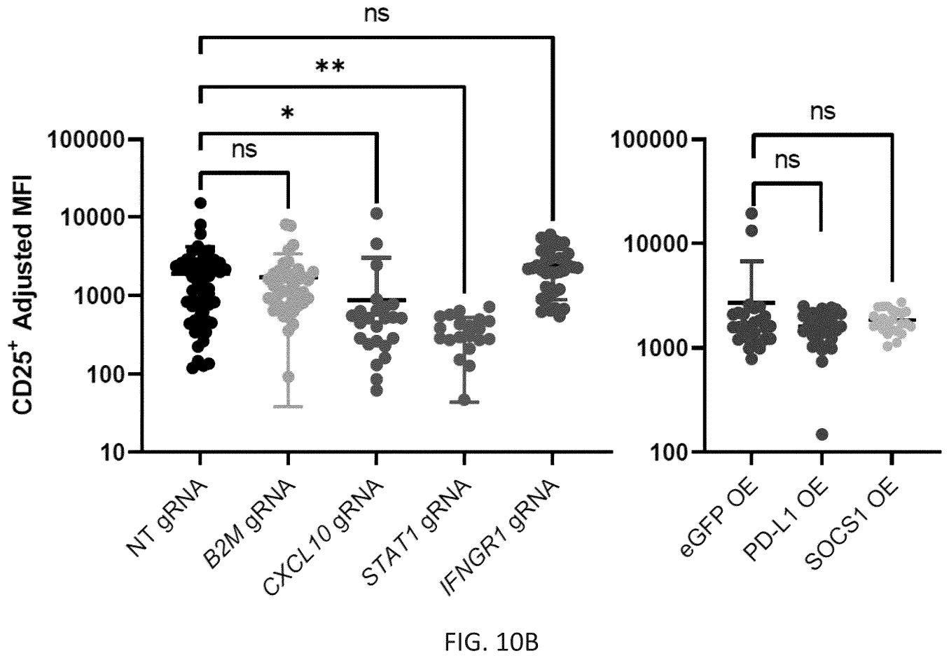

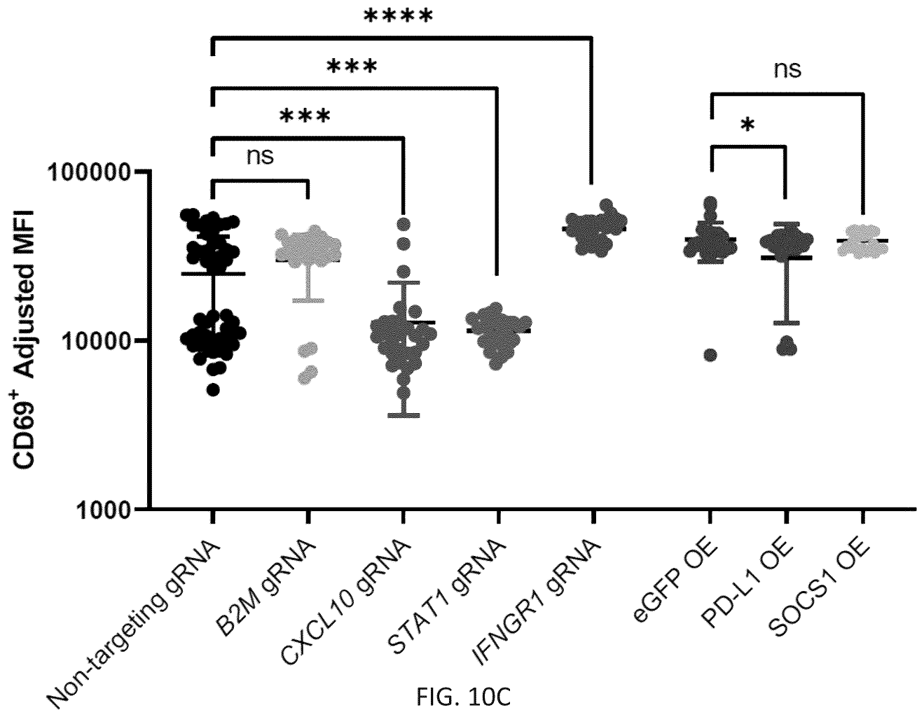

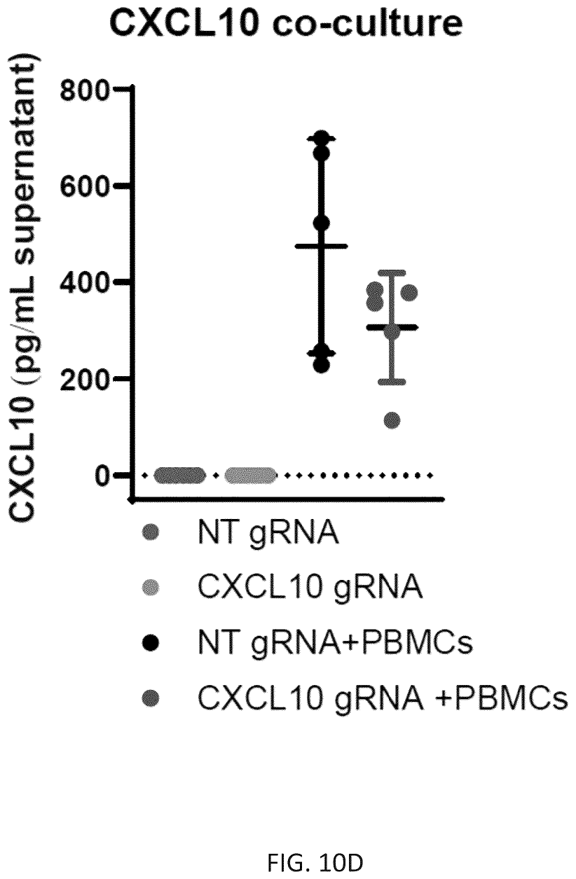

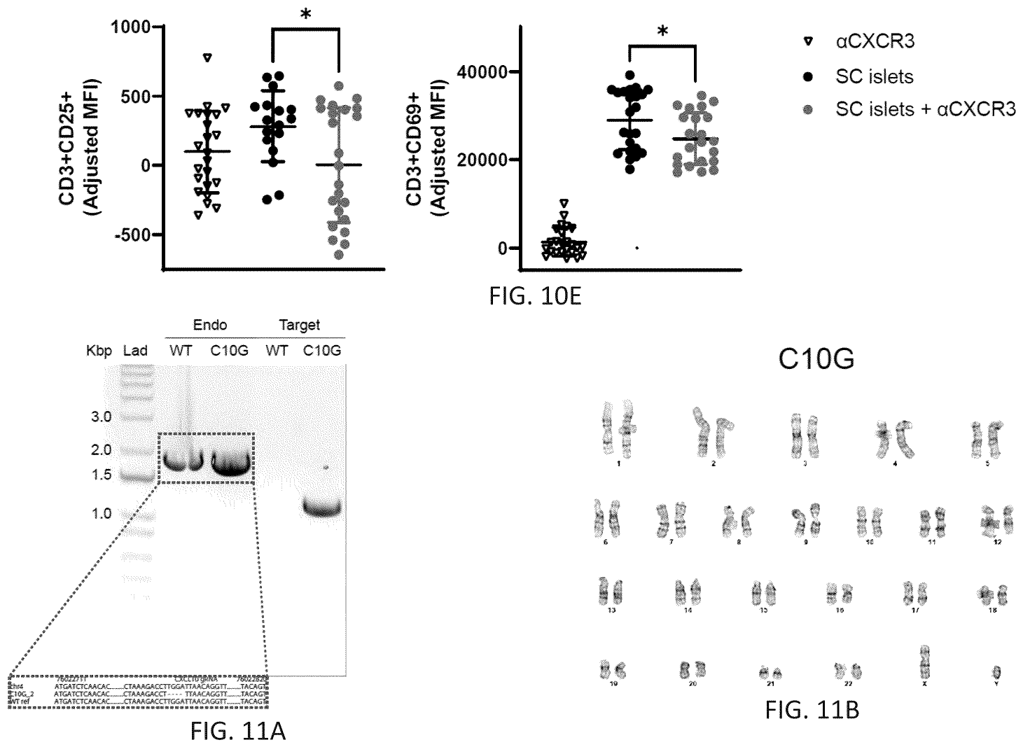

To assess IFN signaling as a target for genetic manipulation, co-culture experiments were run with human allogeneic PBMCs with SC-islets, pre-transduced with lentivirus vectors (FIG. 4B). For gene knockout, vectors expressing Cas9 and gRNAs (in pLentiCRISRv2) were used that target detrimental parts of the IFN pathway: upstream IFNγ receptor alpha chain (IFNGR1), central regulator STAT1, and downstream effectors B2M and CXCL10. For overexpression vectors (in pLX_317) that express open reading frames (ORFs) of intercellular negative regulator SOCS1, downstream immune cell inhibitor PD-L1 (CD274), and CXCL10 (to assess its abundance effect) were chosen. All viral transductions had a perturbing effect on the target protein expression in SC-islets (FIG. 10A) under IFNγ stimuli that fell within the limitations of transduction efficiency. 48 hrs after co-culture, SC-islets were stained for apoptotic markers with the focus on SC-β viability (C-peptide staining) in flow cytometry (FIG. 4C). In parallel, the PBMC fraction was partly stained for T-cell activation markers (FIGS. 10B-10C) or transferred for new cultures to measure proliferation rates after 7 days (FIG. 4D).

B2M knockout, when compared to non-targeting (NT) gRNA transductions, did not reduce SC-islet stimuli of T-cell activation and proliferation, but did reduce the rate of apoptotic SC-β and total SC-islets. In contrast, CXCL10 and STAT1 depletion improved viability of SC-β under immune attack by PBMCs, comparable to the known effect of B2M knockout and PD-L1 overexpression (FIG. 4C). Both CXCL10 and STAT1 depleted SC-islets reduced co-cultured T-cell activation and proliferation compared to NT gRNA SC-islets (FIG. 4D and FIGS. 10B-10C). Reduced secretion of CXCL10 was observed during co-culture (FIG. 10D).

Although IFNGR1 gRNA transduction decreased SC-β cell death, it surprisingly increased T-cell activation and T-cell proliferation. SC-islet overexpression of SOCS1 had a positive effect on their survival and a profound reduction in co-cultured T-cell proliferation, compared to eGFP overexpression. Proof of CXCL10 harmful effect on SC-β under immune attack can be seen by the large increase of apoptosis in SC-β overexpressing CXCL10.

CXCR3 is a chemokine receptor that is expressed on T helper cells, CD8 T-cells, NK cells and monocytes, and reacts with IFN-inducible chemokines CXCL4, CXCL9, CXCL10 and CXCL11. CXCR3 plays both the traditional role of delivering chemotaxis and cell proliferation signals in IFNγ-induced immune responses (Loetscher et al., 1996) but can also influence effector T cell polarization (Wildbaum et al., 2002). To evaluate the extent of importance of CXCL10-CXCR3 axis in SC-islet immunogenicity, SC-islet+PBMCs co-culture experiments were performed with the addition of a blocking antibody to CXCR3 (FIG. 4E). Anti-CXCR3 Ab treatment (‘αCXCR3’) prior to co-culture with SC-islet, reduced T-cell activation and proliferation and the subsequent SC-β apoptotic effect compared to an unspecific IgG treatment (FIGS. 4F-4G and FIG. 10E). As CXCL10 is not the only chemokine that binds CXCR3, an anti-CXCL10 neutralizing antibody was introduced during co-culture and observed highly improved SC-β viability (FIG. 4F). This indicates that CXCL10 contribution to SC-β cell death is substantial. As CXCL10 was suggested to induce apoptosis through binding to TLR4 in β-cells (Schulthess et al., 2009), SC-islets were pre-treated with a TLR4 blocking antibody, before co-culturing with PBMCs. TLR4 antibody treated SC-islets showed a non-significant reduction in apoptotic events (FIG. 4F). This may not rule out TLR4 role entirely but implies that the main contributors to CXCL10-mediated SC-islet killing are CXCR3-induced immune cells.

Generating gene modified hPSC lines comes with the risk of off-target effects and the challenge of acquiring clones that lose their preferable differentiation ability. These obstacles will exponentially increase with the need to edit multiple genes with multiple rounds of transfections and clonal isolations. Therefore, there was a goal to obtain hypo-immunogenic SC-islets with single perturbations. Data gained in this study by scRNA-seq and CRISPR screen established that IFN pathways should be silenced in order to acquire hypo-immunogenicity. Thus, two Hues8 hESCs CRISPR knockout lines, CXCL10 KO and STAT1 KO, were chosen to be generated with the rationale of diminishing the entire IFN signaling through a master regulator (STAT1) or by confinement to the downstream effect (CXCL10).

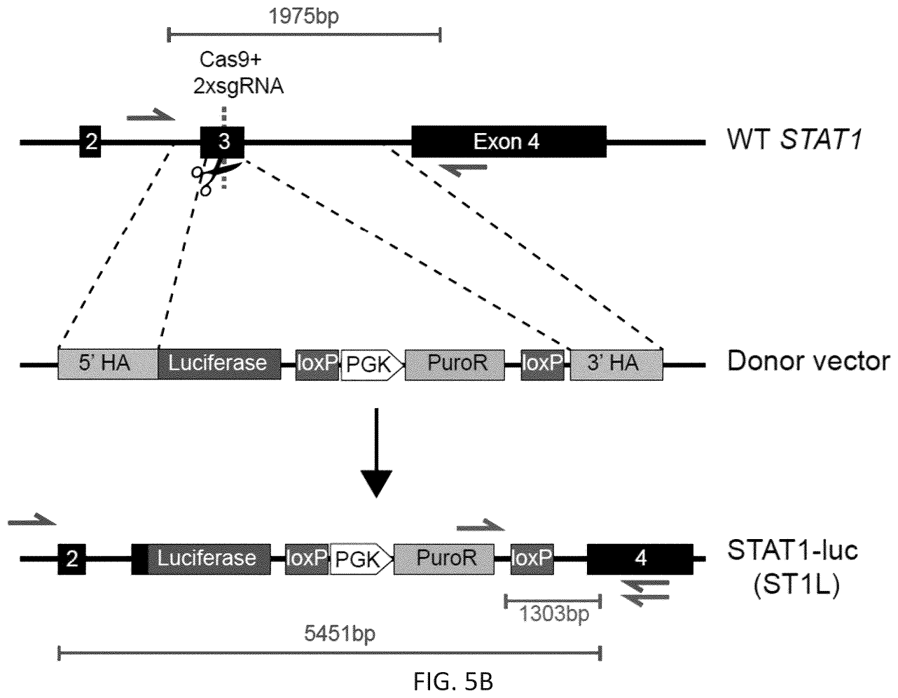

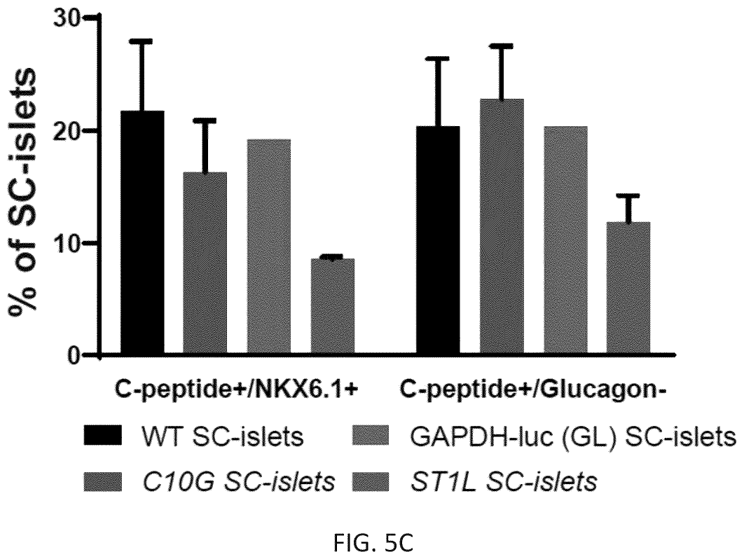

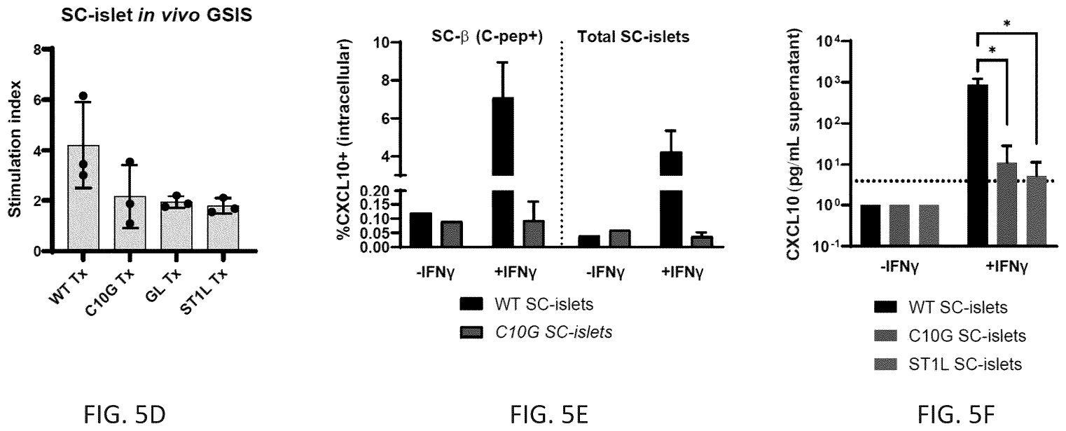

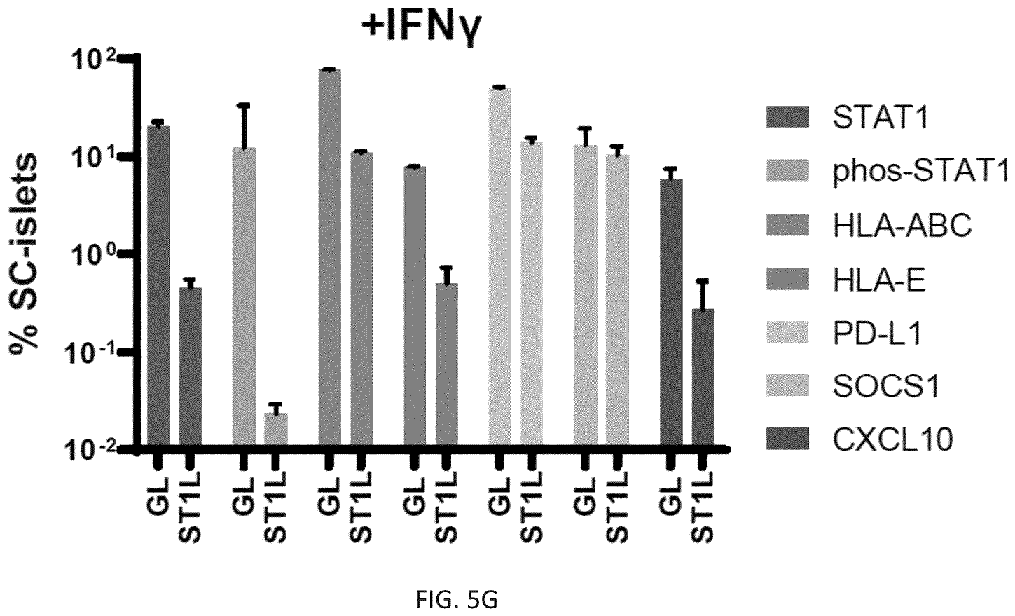

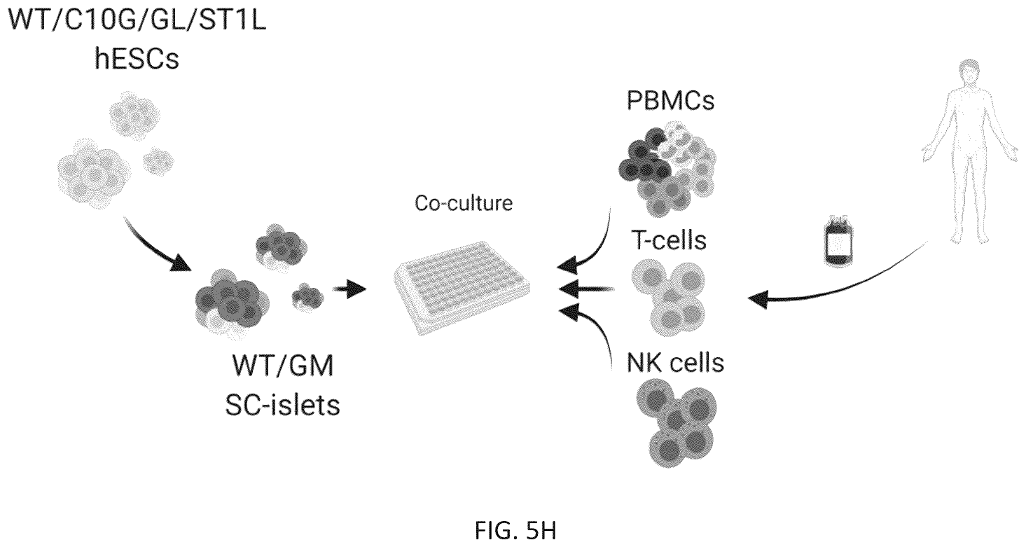

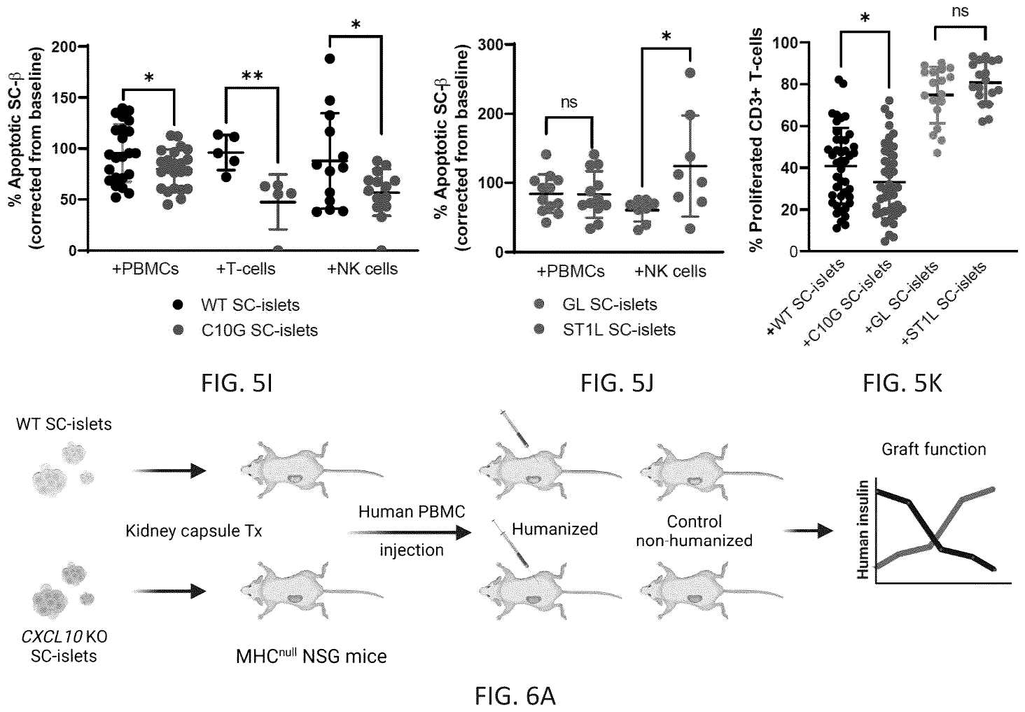

Lines were generated by homology directed repair (HDR), via nucleofection of a Cas9/sgRNA ribonucleoprotein complex (RNP) and a targeting vector. The targeting vector was designed to facilitate the in-frame integration of GFP or luciferase cassettes with puromycin resistance into exon 2 or exon 3 of the CXCL10 or STAT1 loci, respectively (FIGS. 5A-5B). Several heterozygous clones were acquired from each knockout, and a CXCL10-GFP (C10G) clone and a STAT1-luciferase (ST1L) clone were chosen that contained the integrated transgene in one allele along with a nonhomologous end-joining (NHEJ) mutation in the intact endogenous allele, determined by PCR and Sanger sequencing (FIG. 10A). Clones displayed normal karyotyping (FIG. 10B) and pluripotency marker expression (FIG. 10C). For the following experiments, these clones were compared to a wild type Hues8 batch (WT) and a luciferase expressing Hues8 line (GAPDH-luc; GL) (Gerace et al., 2021). C10G, ST1L and control lines were differentiated successfully into the SC-β using the Melton lab β-cell differentiation protocol (Pagliuca et al., 2014; Veres et al., 2019) (FIG. 5C and FIG. 10D) and exhibited positive glucose-stimulated insulin secretion in transplanted mice (FIG. 5D).

As intended, SC-islet differentiated C10G had neglectable intracellular CXCL10 staining and hardly detectable CXCL10 secretion (ELISA), with and without IFNγ stimulation (FIGS. 5E-5F). Less than 1% of ST1L SC-islets were STAT1 positive and even less stained for the activated phosphorylated STAT1 under IFNγ treatment (FIG. 5G). As a regulator of the IFN pathway, the absence of STAT1 in ST1L also led to desensitization to IFNγ, manifested by downregulation of downstream activatory components as HLA proteins and CXCL10 as well as inhibitory (HLA-E, PD-L1, and SOCS1) (FIGS. 5F-5G). ST1L also displayed in frame luciferase activity throughout the differentiation stages, yet reduced over differentiation (FIG. 10E) and lower than GL at Stage 6 (FIG. 10F).

In vitro co-culture assays are powerful tools to predict outcomes of immune attacked cells upon transplantation. Therefore, gene modified (GM) and control SC-islets were co-cultured with allogeneic PBMCs. To evaluate the contribution of specific immune populations on SC-islet killing, GM SC-islets were co-cultured with blood purified T-cells (C10G only) and NK cells (FIG. 5H). Compared to WT, C10G co-cultures displayed remarkable protective performances against allo-PBMCs, T-cells and NK cells in terms of improved SC-β (FIG. 5I) and SC-islet (FIG. 10G) viability and reduced activation and proliferation of T-cells (FIG. 5K and FIG. 10H) in co-cultured PBMCs. In contrast, ST1L displayed minor to poor protectivity against co-cultured PBMCs while significantly more SC-islets were apoptotic after NK cell co-culture (FIG. 5J and FIG. 10G). T-cells from ST1L co-cultured PBMCs did not get activated or proliferate more than those co-cultured with control GL SC-islets (FIG. 5K and FIG. 10I). NK cell activation did not reduce significantly both after C10G and ST1L co-cultures (FIG. 10J). The fact that ST1L does not show lower immunogenicity in co-cultures may be attributed to the lack of IFN-induced negative regulation (discussed above and in FIG. 5G) by PD-L1 and SOCS1.

### CXCL10 Depleted SC-Islets are Hypoimmunogenic in In Vivo Allo-Rejection Models

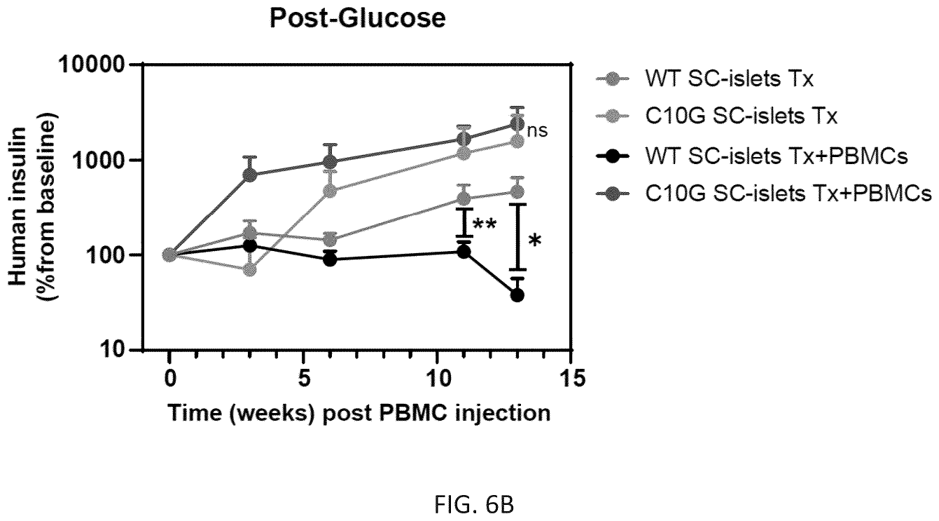

Since ST1L shows ambivalent results in co-culture experiments, nullC10G was focused on in in vivo studies. In a similar humanized mouse (MHC NSG) model as used in FIG. 1 and FIG. 3, C10G or WT SC-islets were transplanted, followed by PBMCs injection from two human donors, leaving 3 mice of each group as non-humanized controls. Graft function was continuously monitored by human insulin ELISA of plasma samples from overnight fasted mice (30 minutes after glucose administration). Starting from week 11 after PBMC injection, graft failure has been observed in humanized mice with WT SC-islets, continuing through week 13 and while control grafts remained functional. Interestingly, C10G SC-islet graft insulin levels remained stable and growing through time, with no apparent difference between humanized (+PBMCs) and control mice (FIG. 6A and FIG. 11A). Therefore, SC-islets with impaired ability to express and secrete CXCL10 are cable to evade immune attack and allograft rejection.

### Discussion

In the pursuit to protect insulin producing SC-β cells from immune destruction this study applied two main approaches to reveal the core players which drive SC-islet immunogenicity: one that observes the occurring events upon immune challenges (using transcriptomics) and the other that filter causes of those events (CRISPR screening) into the most practical mechanism to gain immune-protection. In other words, answering the question of what happens and what can be done to reverse it?

The strongest effect that can be seen in SC-islets, confronted with allogeneic immune cells, is the upregulation of interferon stimulated genes (ISGs). Data shows that T-cells are activated in those co-culture/graft environments and express IFNγ, among many other inflammatory genes. The secreted IFNγ bind to receptors on SC-islets, and lead to an inflammatory cascade in which ISGs are upregulated. A plausible explanation for T-cell activation is antigen presentation through MHC class I molecules, evident by differential expression of B2M, HLA genes and antigen processing genes in SC-islets, in vivo (FIG. 1) and in vitro (FIG. 2). Secreted cytokines by primed T-cells activate more immune cells including NK cells. But the most striking observation was the involvement of chemokines, secreted by SC-islets. CXCL10, has 8-fold higher expression in co-cultured SC-β cells (with PBMCs) compared to SC-β cells alone. Grafted CXCL10-depleted SC-islet cells under allogeneic immune attack have higher chances of survival compared to surrounding cells with other perturbations, as judged by the in vivo CRISPR screen results (FIG. 3). Furthermore, evidence from in vitro and in vivo immune attack models prove that CXCL10 deficient SC-islets are immune evasive compared to wild type cells (FIGS. 4-6).

CXCL10 is one of the most upregulated among cytokines and chemokines in primary human islets (Eizirik et al., 2012) as well as hPSC derived islets (Demine et al., 2020; Dettmer et al., 2022), under exposure to the pro-inflammatory cytokines. It is known as a chemoattractant of NK cells (Ali et al., 2021), monocytes and activated T cells (Taub et al., 1993) through CXCR3 binding (Loetscher et al., 1996). Evidence from islets of recent-onset T1D shows expression of CXCL10 expression in insulitis affected regions with infiltrating lymphocytes that express CXCR3 (Roep et al., 2010; Uno et al., 2010). The results show that CXCL10 induction is not exclusive to SC-β cells but also differentially expressed by other stem cell derived endocrine cells, SC-α and SC-EC cells. This can be interrelated with a recent study, showing evidence of the contribution of pancreatic α-cells to CXCL10 expression in NOD mice and recent onset T1D islets (Nigi et al., 2020).

In a previous study, using a T1D autologous in vitro model CXCL10 was found to be highly secreted from iPSC-islets during co-culture with matched T1D PBMCs (Leite et al., 2020). In the current study, CXCL10 expression was seen mostly in co-cultures (FIGS. 2F-2H) but not in late stages of graft rejections (FIG. 7D and FIG. 7F), certifying CXCL10 as a first responder ‘alarm’ protein, whose effect is crucial in the beginning of SC-islet interactions with a hostile immune system and set the fate for SC-islet survival (FIG. 3, in vivo CRISPR screen). In T1D, islet CXCL10 expression occurs in early stages (Roep et al., 2010; Uno et al., 2010) and levels of CXCL10 in patient's serum is mostly elevated in recent onset compared to established patients (auto-Ab+) (Shimada et al., 2001). Mouse islet isografts expressed high degrees of CXCL10 at day 2 after transplantation into diabetic C57BL/6 mice, but in a lesser degree by day 100 (Bender et al., 2017). Analysis of plasma samples from human islet transplant patients revealed that CXCL10 was among the highest released inflammatory mediators, that peaked 24 hrs post transplantation (Yoshimatsu et al., 2017).

The CRISPR screen in humanized mouse reveals CXCL10 as a harmful gene for graft viability, mostly under human T-cell infiltration but also under standard implantation conditions (FIG. 3C). These stressful conditions might induce an inflammatory response manifested by CXCL10. Paracrine or autocrine CXCL10 was suggested to mediate β-cell failure and apoptosis via CXCR3 (Javeed et al., 2021) or TLR4 binding (Schulthess et al., 2009) on β-cells. This might be an explanation for enrichment of CXCL10 gRNAs compared to intergenic gRNA transduced cells in mice grafts (FIG. 3C), regardless of the human T-cells activity. The co-culture system using receptor blocking antibodies, could only provide evidence for the conventional PBMC-mediated cell death via the CXCL10-CXCR3 axis (FIGS. 4E-4G).

In T1D, pancreatic islets were shown to react to pro-inflammatory cytokines by NF-κB and STAT1 regulation and are part of the immune destruction mechanism of β-cells (Cnop et al., 2005; Eizirik et al., 2012). Although the setting was allogeneic, both transcription factors were upregulated in SC-islets, but only STAT1 depletion showed up as a hit in the CRISPR screen (FIG. 3). When STAT1 gRNAs were transduced by lentiviruses, SC-islets were rescued from immune destruction (FIGS. 4C-4D). However, when a pure line of STAT1 KO (ST1L)SC-islets were used in the same co-culture setting, this rescue was not reproduced (FIGS. 5I-5K). The reason might derive from the efficiency of lentivirus transduction. STAT1 deficient SC-islets lose immune inducing elements such as HLA molecules and CXCL10, but also suffer from loss of immune inhibitory functions like PD-L1 and SOCS1 (FIG. 5G). What consisted as a portion of cells among unperturbed cells (given lentivirus efficiency), STAT1 deficient cells might lower the overall inflammatory response together with unperturbed cells that retain their endogenous inhibitory ligand function.

Downstream to STAT1 is the transcription factor IRF1 that was reported to lead to some opposite effects in β-cells through the induction of SOCS1 (Moore et al., 2011). SOCS1 and PTPN2 are negative regulators of cytokine signaling (Chong et al., 2002; Elvira et al., 2022; Moore et al., 2009) and are both associated with T1D risk loci (Onengut-Gumuscu et al., 2015; Ram and Morahan, 2017). Previous reports have shown that SOCS1 overexpression in NOD mice islets prevent diabetes (Flodstrom-Tullberg et al., 2003), and delays allogeneic islet graft rejection in mouse models (Solomon et al., 2011). The data show that under PBMCs+SC-islet inflammatory responses (humanized mice grafts or co-culture) both IRF1 and SOCS1 are differentially upregulated (FIGS. 1-2). SOCS1 KO SC-islets were depleted in the CRISPR screen in humanized mouse grafts, among PTPN2 KO and PTPRA KO, another PTP family member (Stanley et al., 2015) (FIG. 5D). The effect of SOCS1 overexpression was also later validated and shown as pro-survival in co-cultured SC-islets (FIGS. 4C-4D).

Based on STAT1 KO line (ST1L) ambivalent results (FIG. 5), it was concluded that similar pan-JAK/STAT diminishing strategies should be considered carefully. These approaches include SOCS1 overexpression and IFNGR1 depletions. Although they proved potent for SC-islet immune protection in co-culture experiments (FIGS. 4C-4D), pure transgenic lines of SOCS1 OE or IFNGR1 KO might have consequences of losing the inflammatory negative regulation feedback of JAK/STAT signaling. PD-L1 downregulation under JAK/STAT silencing will expose SC-islets to T-cell attack, while HLA downregulation will draw NK cell killing. Such stem cell lines may be further edited with additional modification(s) that will address these concerns.

### REFERENCES

Alagpulinsa, D. A., Cao, J. J. L., Driscoll, R. K., Sirbulescu, R. F., Penson, M. F. E., Sremac, M., Engquist, E. N., Brauns, T. A., Markmann, J. F., Melton, D. A., et al. (2019). Alginate microencapsulation of human stem cell-derived beta cells with CXCL12 prolongs their survival and function in immunocompetent mice without systemic immunosuppression. Am J Transplant 19, 1930-1940.
Banting, F. G., Best, C. H., Collip, J. B., Campbell, W. R., and Fletcher, A. A. (1922). Pancreatic Extracts in the Treatment of Diabetes Mellitus. Can Med Assoc J 12, 141-146.
Barrett, J. C., Clayton, D. G., Concannon, P., Akolkar, B., Cooper, J. D., Erlich, H. A., Julier, C., Morahan, G., Nerup, J., Nierras, C., et al. (2009). Genome-wide association study and meta-analysis find that over 40 loci affect risk of type 1 diabetes. Nat Genet 41, 703-707.
Brehm, M. A., Kenney, L. L., Wiles, M. V., Low, B. E., Tisch, R. M., Burzenski, L., Mueller, C., Greiner, D. L., and Shultz, L. D. (2019). Lack of acute xenogeneic graft- versus-host disease, but retention of T-cell function following engraftment of human peripheral blood mononuclear cells in NSG mice deficient in MHC class I and II expression. FASEB J 33, 3137-3151.
Cai, E. P., Ishikawa, Y., Zhang, W., Leite, N. C., Li, J., Hou, S., Kiaf, B., Hollister-Lock, J., Yilmaz, N. K., Schiffer, C. A., et al. (2020). Genome-scale in vivo CRISPR screen identifies RNLS as a target for beta cell protection in type 1 diabetes. Nat Metab 2, 934-945.
Caspi, A., Sugden, K., Moffitt, T. E., Taylor, A., Craig, I. W., Harrington, H., McClay, J., Mill, J., Martin, J., Braithwaite, A., et al. (2003). Influence of life stress on depression: moderation by a polymorphism in the 5-HTT gene. Science 301, 386-389.
Castro-Gutierrez, R., Alkanani, A., Mathews, C. E., Michels, A., and Russ, H. A. (2021). Protecting Stem Cell Derived Pancreatic Beta-Like Cells From Diabetogenic T Cell Recognition. Front Endocrinol (Lausanne) 12, 707881.
Deuse, T., Hu, X., Agbor-Enoh, S., Jang, M. K., Alawi, M., Saygi, C., Gravina, A., Tediashvili, G., Nguyen, V. Q., Liu, Y., et al. (2021). The SIRPalpha-CD47 immune checkpoint in NK cells. J Exp Med 218.
Deuse, T., Hu, X., Gravina, A., Wang, D., Tediashvili, G., De, C., Thayer, W. O., Wahl, A., Garcia, J. V., Reichenspurner, H., et al. (2019). Hypoimmunogenic derivatives of induced pluripotent stem cells evade immune rejection in fully immunocompetent allogeneic recipients. Nat Biotechnol 37, 252-258.
Doench, J. G. (2018). Am I ready for CRISPR?A user's guide to genetic screens. Nat Rev Genet 19, 67-80.
Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize off-target effects of CRISPR-Cas9. Nat Biotechnol 34, 184-191.
Espino-Paisan, L., de la Calle, H., Fernandez-Arquero, M., Figueredo, M. A., de la Concha, E. G., Urcelay, E., and Santiago, J. L. (2011). A polymorphism in PTPN2 gene is associated with an earlier onset of type 1 diabetes. Immunogenetics 63, 255-258.
Galic, S., Sachithanandan, N., Kay, T. W., and Steinberg, G. R. (2014). Suppressor of cytokine signalling (SOCS) proteins as guardians of inflammatory responses critical for regulating insulin sensitivity. Biochem J 461, 177-188.
Gerace, D., Boulanger, K. R., Hyoje-Ryu Kenty, J., and Melton, D. A. (2021). Generation of a heterozygous GAPDH-Luciferase human ESC line (HVRDe008-A-1) for in vivo monitoring of stem cells and their differentiated progeny. Stem Cell Res 53, 102371.
Gornalusse, G. G., Hirata, R. K., Funk, S. E., Riolobos, L., Lopes, V. S., Manske, G., Prunkard, D., Colunga, A. G., Hanafi, L. A., Clegg, D. O., et al. (2017). HLA-E-expressing pluripotent stem cells escape allogeneic responses and lysis by NK cells. Nat Biotechnol 35, 765-772.
Gurzov, E. N., Stanley, W. J., Brodnicki, T. C., and Thomas, H. E. (2015). Protein tyrosine phosphatases: molecular switches in metabolism and diabetes. Trends Endocrinol Metab 26, 30-39.
Gurzov, E. N., Stanley, W. J., Pappas, E. G., Thomas, H. E., and Gough, D. J. (2016). The JAK/STAT pathway in obesity and diabetes. FEBS J 283, 3002-3015.
Han, X., Wang, M., Duan, S., Franco, P. J., Kenty, J. H., Hedrick, P., Xia, Y., Allen, A., Ferreira, L. M. R., Strominger, J. L., et al. (2019). Generation of hypoimmunogenic human pluripotent stem cells. Proc Natl Acad Sci USA 116, 10441-10446.
Kharbikar, B. N., Chendke, G. S., and Desai, T. A. (2021). Modulating the foreign body response of implants for diabetes treatment. Adv Drug Deliv Rev 174, 87-113.
Kovatchev, B. (2019). A Century of Diabetes Technology: Signals, Models, and Artificial Pancreas Control. Trends Endocrinol Metab 30, 432-444.
Lehmann, R., Spinas, G. A., Moritz, W., and Weber, M. (2008). Has time come for new goals in human islet transplantation? Am J Transplant 8, 1096-1100.
Leite, N. C., Sintov, E., Meissner, T. B., Brehm, M. A., Greiner, D. L., Harlan, D. M., and Melton, D. A. (2020). Modeling Type 1 Diabetes In Vitro Using Human Pluripotent Stem Cells. Cell Rep 32, 107894.
Loetscher, M., Gerber, B., Loetscher, P., Jones, S. A., Piali, L., Clark-Lewis, I., Baggiolini, M., and Moser, B. (1996). Chemokine receptor specific for IP10 and mig: structure, function, and expression in activated T-lymphocytes. J Exp Med 184, 963-969.
Moore, F., Naamane, N., Colli, M. L., Bouckenooghe, T., Ortis, F., Gurzov, E. N., Igoillo-Esteve, M., Mathieu, C., Bontempi, G., Thykjaer, T., et al. (2011). STAT1 is a master regulator of pancreatic {beta}-cell apoptosis and islet inflammation. J Biol Chem 286, 929-941.
Nostro, M. C., Sarangi, F., Yang, C., Holland, A., Elefanty, A. G., Stanley, E. G., Greiner, D. L., and Keller, G. (2015). Efficient generation of NKX6-1+ pancreatic progenitors from multiple human pluripotent stem cell lines. Stem Cell Reports 4, 591-604.
Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
Parent, A. V., Faleo, G., Chavez, J., Saxton, M., Berrios, D. I., Kerper, N. R., Tang, Q., and Hebrok, M. (2021). Selective deletion of human leukocyte antigens protects stem cell-derived islets from immune rejection. Cell Rep 36, 109538.
Raffin, C., Vo, L. T., and Bluestone, J. A. (2020). Treg cell-based therapies: challenges and perspectives. Nat Rev Immunol 20, 158-172.
Ramzy, A., Thompson, D. M., Ward-Hartstonge, K. A., Ivison, S., Cook, L., Garcia, R. V., Loyal, J., Kim, P. T. W., Warnock, G. L., Levings, M. K., et al. (2021).
Implanted pluripotent stem-cell-derived pancreatic endoderm cells secrete glucose-responsive C-peptide in patients with type 1 diabetes. Cell Stem Cell 28, 2047-2061 e2045.
Rezania, A., Bruin, J. E., Arora, P., Rubin, A., Batushansky, I., Asadi, A., O'Dwyer, S., Quiskamp, N., Mojibian, M., Albrecht, T., et al. (2014). Reversal of diabetes with insulin producing cells derived in vitro from human pluripotent stem cells. Nat Biotechnol 32, 1121-1133.
Russ, H. A., Parent, A. V., Ringler, J. J., Hennings, T. G., Nair, G. G., Shveygert, M., Guo, T., Puri, S., Haataja, L., Cirulli, V., et al. (2015). Controlled induction of human pancreatic progenitors produces functional beta-like cells in vitro. EMBO J 34, 1759-1772.
Schulthess, F. T., Paroni, F., Sauter, N. S., Shu, L., Ribaux, P., Haataja, L., Strieter, R. M., Oberholzer, J., King, C. C., and Maedler, K. (2009). CXCL10 impairs beta cell function and viability in diabetes through TLR4 signaling. Cell Metab 9, 125-139.
Shapiro, A. M., Lakey, J. R., Ryan, E. A., Korbutt, G. S., Toth, E., Warnock, G. L., Kneteman, N. M., and Rajotte, R. V. (2000). Islet transplantation in seven patients with type 1 diabetes mellitus using a glucocorticoid-free immunosuppressive regimen. N Engl J Med 343, 230-238.
Shultz, L. D., Keck, J., Burzenski, L., Jangalwe, S., Vaidya, S., Greiner, D. L., and Brehm, M. A. (2019). Humanized mouse models of immunological diseases and precision medicine. Mamm Genome 30, 123-142.
Solomon, M., Flodstrom-Tullberg, M., and Sarvetnick, N. (2011). Beta-cell specific expression of suppressor of cytokine signaling-1 (SOCS-1) delays islet allograft rejection by down-regulating Interferon Regulatory Factor-1 (IRF-1) signaling. Transpl Immunol 24, 181-188.
Stanley, W. J., Litwak, S. A., Quah, H. S., Tan, S. M., Kay, T. W., Tiganis, T., de Haan, J. B., Thomas, H. E., and Gurzov, E. N. (2015). Inactivation of Protein Tyrosine Phosphatases Enhances Interferon Signaling in Pancreatic Islets. Diabetes 64, 2489-2496.
Veres, A., Faust, A. L., Bushnell, H. L., Engquist, E. N., Kenty, J. H., Harb, G., Poh, Y. C., Sintov, E., Gurtler, M., Pagliuca, F. W., et al. (2019). Charting cellular identity during human in vitro beta-cell differentiation. Nature 569, 368-373.
Wang, D., Quan, Y., Yan, Q., Morales, J. E., and Wetsel, R. A. (2015). Targeted Disruption of the beta2-Microglobulin Gene Minimizes the Immunogenicity of Human Embryonic Stem Cells. Stem Cells Transl Med 4, 1234-1245.
Wei, Z., Yoshihara, E., He, N., Hah, N., Fan, W., Pinto, A. F. M., Huddy, T., Wang, Y., Ross, B., Estepa, G., et al. (2018). Vitamin D Switches BAF Complexes to Protect beta Cells. Cell 173, 1135-1149 e1115.
Wildbaum, G., Netzer, N., and Karin, N. (2002). Plasmid DNA encoding IFN-gamma inducible protein 10 redirects antigen-specific T cell polarization and suppresses experimental autoimmune encephalomyelitis. J Immunol 168, 5885-5892.
Xu, H., Wang, B., Ono, M., Kagita, A., Fujii, K., Sasakawa, N., Ueda, T., Gee, P., Nishikawa, M., Nomura, M., et al. (2019). Targeted Disruption of HLA Genes via CRISPR-Cas9 Generates iPSCs with Enhanced Immune Compatibility. Cell Stem Cell 24, 566-578 e567.
Yoshihara, E., O'Connor, C., Gasser, E., Wei, Z., Oh, T. G., Tseng, T. W., Wang, D., Cayabyab, F., Dai, Y., Yu, R. T., et al. (2020). Immune-evasive human islet-like organoids ameliorate diabetes. Nature 586, 606-611.

1. A modified stem cell-derived beta cell comprising one or more perturbations in the JAK/STAT Type II interferon (IFN) pathway.

2. The modified stem cell-derived beta cell of claim 1, wherein the perturbation occurs in an IFNγ signaling mediator or a downstream component of the IFN pathway.

3. The modified stem cell-derived beta cell of claim 1, wherein the perturbation occurs in a downstream inflammatory component of the IFN pathway.

4. The modified stem cell-derived beta cell of claim 1, wherein the perturbation comprises reduced expression of one or more downstream components of the IFN pathway.

5. The modified stem cell-derived beta cell of claim 1, wherein the perturbation comprises eliminated expression of one or more downstream components of the IFN pathway.

6. The modified stem cell-derived beta cell of claim 1, wherein the perturbation comprises reduced expression of one or more genes listed in Table 1.

7. The modified stem cell-derived beta cell of claim 1, wherein the perturbation comprises eliminated expression of one or more genes listed in Table 1.

8. The modified stem cell-derived beta cell of claim 1, wherein the perturbation comprises reduced expression of one or more genes selected from the group consisting of CXCL10, STAT1, and TAP1.

9. A modified stem cell-derived beta cell modified to eliminate expression of CXCL10.

10. A modified stem cell-derived beta cell modified to eliminate expression of STAT1.

11. The modified stem cell-derived beta cell of claim 9, wherein the modified stem cell-derived beta cell is a hypoimmunogenic stem cell-derived beta cell.

12. The modified stem cell-derived beta cell of claim 9, wherein the modified stem cell-derived beta cell exhibits auto and/or allogeneic rejection protection.

13. The modified stem cell-derived beta cell of claim 9, wherein the modified stem cell-derived beta cell exhibits increased cell survival under immune rejection.

14. The modified stem cell-derived beta cell of claim 9, wherein the modified stem cell-derived beta cell exhibits decreased T cell activation and/or NK cell activation upon transplant.

15. The modified stem cell-derived beta cell of claim 9, further comprising increased or activated expression of one or more immunomodulatory factors.

16. The modified stem cell-derived beta cell of claim 15, wherein the one or more immunomodulatory factors are selected from Table 2.

17. The modified stem cell-derived beta cell of claim 15, wherein the one or more immunomodulatory factors are selected from the group consisting of PD-L1, CD47, SOCS1, and HLA-E.

18. The modified stem cell-derived beta cell of claim 9, wherein the beta cell is a human cell.

19. The modified stem cell-derived beta cell of claim 9, wherein the beta cell is a non-human cell.

20. The modified stem cell-derived beta cell of claim 19, wherein the non-human cell is a mouse cell.

21. A method of producing a hypoimmunogenic stem cell-derived beta cell comprising silencing the IFN pathway of the beta cell.

22. (canceled)

23. (canceled)

24. (canceled)

25. (canceled)

26. A method of producing a hypoimmunogenic stem cell-derived beta cell comprising perturbing one or more downstream components of the IFN pathway of the beta cell.

27.-41. (canceled)
